(19) United States
(12) Patent Application Publication
(10) Pub. No.: US 2006/0241130 A1
(43) Pub. Date: Oct. 26, 2006

(54) Anti-inflammatory compositions and uses thereof

(76) Inventors: Ehud Keinan, Timrat (IL); Aron Alt, Haifa (IL)

Correspondence Address: Martin Moynihan;Anthony Castorina, Suite 207, 2001 Jefferson Davis Highway, Arlington, VA, 22202, US

(21) Appl. No.: 10/543,590

(22) PCT Filed: Feb. 1, 2004
(86) PCT No.: PCT/IL04/00096

§ 371 Date: Feb. 7, 2006

Related U.S. Application Data
(60) Provisional application No. 60/443,866, filed on Jan. 31, 2003.
(60) Provisional application No. 60/453,213, filed on Mar. 11, 2003.

(51) Int. Cl. A61K 31/522; A61K 31/404; A61K 31/202; A61K 31/353; A61K 31/01; A61K 31/045; A61K 31/015

(52) U.S. Cl. 514/263.31; 514/456; 514/414; 514/560; 514/762; 514/763; 514/724; 514/729

(57) ABSTRACT

Methods of treating medical conditions associated with inflammation employing compounds capable of inhibiting an activity and/or a formation of an oxidant associated with the inflammation, pharmaceutical composition and inhalation devices containing such compounds are provided. Further provided are methods of identifying drug candidates for treating inflammation-associated medical conditions by inhibiting an activity and/or a formation of an oxidant associated with the inflammation and methods of diagnosing such medical conditions.

### FIELD AND BACKGROUND OF THE INVENTION

The present invention relates to pharmaceutical compositions, methods and devices for treating inflammation-associated medical conditions such as asthma. The present invention further relates to methods of identifying drug candidates for treating inflammation-associated medical conditions and to methods of diagnosing such medical conditions.

Inflammation-associated medical conditions include numerous highly debilitating and/or lethal diseases, such as allergic, infectious, autoimmune, inflammatory transplantation-related, malignant, degenerative, and/or idiopathic diseases. To date, however, no satisfactory treatment and/or prevention methods are currently available for treatment of such medical conditions.

For example, allergic diseases, such as asthma, and allergies to seasonal pollens, ragweed, dust mites, pet fur, cosmetics, insect bites, and various foods are significantly debilitating to a large proportion of the population, can be fatal, and are of great economic significance due to the large market for allergy drugs. Currently available prevention and/or treatment methods for such allergic diseases remain suboptimal. Inflammatory infectious diseases for which no satisfactory prevention and treatment methods are available include acquired immunodeficiency syndrome (AIDS) caused by human immunodeficiency virus (HIV), influenza, malaria, hepatitis, tuberculosis, cholera, Ebola virus infection, and severe acute respiratory syndrome (SARS). Such inflammatory infectious diseases are collectively responsible for millions of deaths worldwide each year. Ominously, diseases such as AIDS, and antibiotic-resistant bacterial and mycobacterial infections, such as antibiotic-resistant staphylococcal and tuberculosis infections, respectively, to which there are no satisfactory cures for most affected individuals, are on the increase. Also of concern is the widely perceived and anticipated threat of biological warfare using agents causing lethal inflammatory infectious diseases, such as anthrax, smallpox, and bubonic plague. Autoimmune diseases represent a large group of highly debilitating and/or lethal inflammatory diseases which includes major diseases of great clinical and economic impact such as rheumatoid arthritis, type I diabetes and multiple sclerosis. There are no currently available satisfactory methods for preventing and/or treating such diseases. Inflammatory malignant diseases include lethal malignancies such as breast cancer, lung cancer, colorectal cancer, melanoma and prostate cancer which are a tremendous medical and economic burden, particularly in industrialized populations. To date no optimal methods of treating and/or preventing such diseases exist. Inflammatory transplantation-related diseases for which no satisfactory treatment and/or prevention methods are presently available include diseases such as graft rejection and graft-versus-host disease (GVHD). Such diseases are major causes of failure of therapeutic transplantation, a medical procedure of last resort broadly practiced for treating numerous life-threatening diseases, such as cardiac, renal, pulmonary, hepatic and pancreatic failure. Inflammatory degenerative diseases for which there are no presently available optimal treatment and/or prevention methods include highly debilitating and/or lethal diseases such as inflammatory bowel disease, inflammatory cardiovascular diseases such as atherosclerosis, post-operative restenosis, myocarditis, and inflammatory neurological diseases such as Alzheimer's disease, Parkinson's disease, and spongiform encephalopathies. Inflammatory injuries for which no satisfactory treatment and/or prevention methods exist include myocardial infarction, cerebral embolism, smoking associated emphysema, alcohol consumption associated cirrhosis, sunburn, radiation injury, bone fractures, muscle injury, and tendon/ligament injury.

The prevalence of asthma, particularly in the industrialized countries, has increased over the past two decades at an alarming rate (The National Asthma Education and Prevention Program (NAEPP) Expert Panel Report 2: Guidelines for the Diagnosis and Management of Asthma (EPR-2), 1997; A. L. Sheffer, Ed., Global Strategy for Asthma Management and Prevention. NHLBI/WHO Workshop report. National Institutes of Health, National Heart, Lung, and Blood Institute, Publication No. 95-3659, January 1995; U.S. Pat. No. 6,462,020 to Houck et al.). Since approximately 1980, the frequency of this disorder has almost doubled and this disease has become the leading cause of hospitalization among children younger than 15 years (Elias et al., 2003. J. Clin. Invest. 111:291-297). In the United States alone, asthma affects over 15 million people, and more than 5 percent of all children younger than age 18 have experienced asthma attacks. Asthma statistics in the U.S.A. show an annual toll of more than 5,000 deaths, over 100 million lost working days and 470,000 hospitalizations. The annual direct and indirect annual costs of asthma in the USA are estimated at 13 billion dollars.

Asthma is clinical syndrome involving recurrent episodes of wheezing, coughing, dyspnea, chest tightness, and cough associated with bronchoconstriction, hypersecretion and bronchial hyper-responsiveness. In severe cases, asthma may be of sudden onset and fatal. Chronic inflammation associated with asthma may result in airway remodeling, characterized by hypertrophy of smooth muscle, mucus glands and goblet cells, as well as subepithelial fibrosis. A schematic diagram outlining various pathophysiological mechanisms involved in asthma is provided in FIG. 1. Both hereditary and environmental factors, including allergens, viruses and irritants, are involved in the onset of asthma and in its inflammatory exacerbations. More than half of asthmatics (adults and children) have allergies, and, indeed, allergy to house dust mite feces is a major factor in the development of the disease, and in the occurrence of exacerbations. Infection with respiratory syncytial virus during infancy is also highly associated with the development of asthma, and viral respiratory infections often trigger acute episodes. The exact causes of asthma remain unknown, however, recurrent acute and chronic inflammation has become the dominant hypothesis explaining the abnormal behavior of the airways in this disease (Woolcock and Barnes, in: “Asthma,” Barnes et al. Eds., Lippincott-Raven Publishers, Philadelphia 1997, Chapter 1, 3-8; Elias et al., 2003. J. Clin. Invest. 111:291-297). Even patients with mild disease exhibit airway inflammation, including infiltration of the mucosa and epithelium with activated T-cells, mast cells, and eosinophils. Thus, pulmonary inflammation in asthma is thought to arise due to various inflammatory mediators, including cytokines and chemokines, which are released from resident lung cells as well as T-cells and other inflammatory cells that are recruited to the airway. Such mediators promote eosinophil growth and maturation, and IgE-mediated degranulation of mast cells, and lead to increased microvascular permeability (Elias et al., 2003. J. Clin. Invest. 111:291-297), disruption of the epithelium, and stimulation of neural reflexes and mucus secretion (P. Ward, in: “Asthma,” Barnes et al. Eds., Lippincott-Raven Publishers, Philadelphia 1997, Chapter 19, pp. 241-248).

Immunohistochemical analysis in endobronchial biopsy specimens has confirmed that human lung mast cells produce tumor necrosis factor (TNF), IL-4, IL-5, and IL-6 following IgE stimulation in-vitro (Rossi G L. and Olivieri D., 1997. Chest 112:523-29). TNF and IL-4 can potentiate up-regulation in the endothelial layer of the bronchial vasculature of the expression of the immunoglobulin superfamily adhesion molecule vascular cell adhesion molecule (VCAM)-1. Eosinophils, basophils and mononuclear cells display the very late activation antigen (VLA)-4 integrin on their cellular surfaces, which interacts with VCAM-1. Thus, through the interaction VLA-4/VCAM-1, TNF and IL-4 facilitate the recruitment of circulating leukocytes. The capacity of mast cells to release preformed cytokines, such as TNF, in response to IgE-mediated stimulus, or to rapidly synthesize others, such as IL-4, and IL-5, could be the initial event leading to bronchial inflammation. In fact, the induction and activation of Tₕ2 T-cell clones, through a further production of cytokines, facilitates the activation and recruitment of eosinophils, which act as direct effectors of the inflammatory reaction. Tₕ2 T-cells are the subset of T-cells considered to play a central role in mediating allergic reactions, such as atopic asthma. In turn, the cytokines produced by leukocytes (Tₕ2 cells, in particular) profoundly affect the development, activation, and priming of mucosal mast cells, thus promoting a proinflammatory positive feedback loop. The recent findings that human mast cells produce IL-8 and that murine pulmonary-derived mast cells express both chemokines, monocyte chemoattractant protein (MCP)-1 and macrophage inflammatory protein (MIP)-1. This suggests that, besides the cytokines classically involved in leukocyte recruitment, such as IL-4, IL-5, and TNF, mast cells also elaborate additional, potent chemoattractants in the airways, such as IL-8, which act on eosinophils and polymorphonuclear leukocytes. Moreover, because chemokines acting as histamine-releasing factors elicit mast cell degranulation, they may further sustain an autocrine activation loop. Mast cells and basophils are thought to play a key role in B-cell growth since they provide the cell-cell interactions contact that is required, along with IL-4, for IgE synthesis in-vitro. This suggests that mast cells may directly regulate the production of IgE independently of T-cells, and may, upon IgE cross-linking, generate sufficient quantities of IL-4 to initiate local Tₕ2 type responses. Moreover, mast cells can also act as an antigen-presenting cells (APCs) to T-lymphocytes, suggesting an even larger role for mast cells in the pathogenic mechanisms of asthma.

Eicosanoids, a class of compounds which includes leukotrienes, prostaglandins, and thromboxanes, play key roles in asthma pathogenesis. Leukotrienes, of which there are A, B, C, D, and E subtypes, and which are known as the slow reacting substance of anaphylaxis (“SRS-A”), play a crucial role in asthma associated bronchial inflammation. These mediators are involved in triggering phenomena involved in asthma, such as airway smooth muscle spasm, increased vascular permeability, reduced mucociliary transport, and attraction, adhesion and aggregation of various leukocytes, such as neutrophils, eosinophils, and monocytes, to blood vessels. Such mediators therefore lead to interstitial edema, mucus overproduction, and pulmonary bronchospasm. Certain classes of leukotrienes, for example, the cysteinyl leukotrienes (such as LTD4), are particularly potent bronchoconstrictors, being approximately 100 to 1,000 times more active than histamine. Leukotrienes, including cysteinyl leukotrienes, are released from mast cells during degranulation. Like related prostaglandin compounds, leukotrienes are synthesized from arachidonic acid in the cell membrane. Arachidonic acid in mast cells, eosinophils, macrophages, monocytes, and basophils is formed from membrane phospholipids by the activation of phospholipase A2. After its formation, arachidonic acid undergoes metabolism via two major pathways: the cyclooxygenase pathway, which produces various prostaglandins and thromboxanes; and the 5-lipoxygenase pathway which produces leukotrienes in the cytoplasm. A number of anti-leukotrienes that either block leukotriene receptors or prevent leukotriene synthesis by blocking the enzyme 5-lipoxygenase are under investigation and in commercial use. The leukotriene inhibitors are heterogeneous in action, with some blocking 5-lipoxygenase directly, some inhibiting the protein activating 5-lipoxygenase, and some displacing arachidonate from its binding site on the protein. The leukotriene antagonists, by contrast, block the receptors themselves that mediate airway hyperactivity, bronchoconstriction, and hypersecretion.

Currently, asthma is treated with oral and inhaled bronchodilators which are directed at suppressing the symptoms of asthma but which do not affect the underlying inflammatory causes. Bronchodilating beta agonists (selective beta adrenergic receptor agonists) were introduced three decades ago for the treatment of asthma, and while these provide a measure of prophylaxis/palliation of symptoms, they are limited in efficacy and effective duration. Recently, an extra long-acting beta agonist, salmeterol (duration up to 12 hours), was introduced in the United States, however this agent is suboptimally effective and includes the critical drawback that it may mask inflammatory signs, and therefore must be used with an anti-inflammatory. Recognition during the last 10 years of the importance of inflammation in the etiology of asthma has led to the increased use of corticosteroids, however corticosteroids have highly undesirable side-effects, and are suboptimally effective at treating asthma. Moderate asthma is currently treated with a daily inhaled anti-inflammatory-corticosteroid or mast cell inhibitor, cromolyn sodium (CS) or nedocromil, plus an inhaled beta agonist which must be administered 3-4 times per day, but such treatments are usually effective only for asthma that is associated with allergens or exercise and then, typically, only for juvenile asthmatics. Inhaled corticosteroids improve inflammation, airway hyperreactivity, and obstruction, and reduce the number of acute exacerbations. However, it takes a month before effects are apparent and up to a year for marked improvement to occur. The most frequent side effects are hoarseness and oral candidiasis. More serious side effects have been reported, including partial adrenal suppression, growth inhibition, and reduced bone formation. Beclomethasone, triamcinolone, and flunisolide probably have a similar mg-for-mg potency; the newer approvals budesonide and fluticasone are more potent yet retain systemic side effects. New bioactive compounds effective in treatment of inflammatory diseases, such as asthma, having minimal side effects are continually being sought. For example, new drugs have been developed to inhibit mast cell degranulation and activation of other inflammatory cells and pathways (U.S. Pat. No. 6,462,020, to Houck, et al.). Nevertheless, these new drugs target only one or two aspects of the persistent inflammation and not the entire phenomenon, as described in FIG. 1.

Thus, there remains a clear and long-felt need for optimal methods of treating inflammatory diseases such as asthma.

Various lines of evidence have linked the effects of ozone to asthma. Numerous studies investigating the pulmonary toxicity of environmental ozone, a notoriously harmful component of air pollution, have demonstrated that exposure to ozone, even at levels below the present American National Ambient Air Quality Standard, induces airway inflammation and lung injury in both humans and animals (Lippmann, 1989. J. Air Poll. Control Assoc. 39:672-695). Inhaled ozone has been found to exert an enhancing effect on allergic lung sensitization when mice are exposed to an aerosolized allergen (Osebold et al., 1988. Proc. Soc. Exp. Biol. Med. 188:259-264). It has been demonstrated that ozone can shift helper T-cell effector responses towards Tₕ2 type (allergic response specific) responses (Neuhaus-Steinmetz, F. et al., 2000. Am. J. Respir. Cell. Mol. Biol. 23:228-233). Ozone-induced pulmonary inflammation has been shown to be associated with upregulation of inflammatory mediators and activation of inflammatory cells. For example, significant pulmonary neutrophilia and increased levels of proinflammatory mediators, such as IL-6, IL-8, and leukotriene have consistently been found in bronchoalveolar lavage fluid (BALF) and in bronchial mucosal biopsies of healthy humans, and particularly in asthmatic patients, exposed to low levels of ozone (Basha et al., 1994. Chest, 106:1757-1765; Nightingale et al., 1999. Thorax 54:1061-1069; M. J. Coffey et al., 1996. Toxicology 114:187-197; Bascom et al., 1996. Am. J. Respir. Crit. Care Med. 153:3-50). It has also been demonstrated that sensory neuropeptides such as tachykinins, including substance P and neurokinin A, which are present in neurons and inflammatory cells in the airway, are released following exposure to ozone (Joos et al., 2000. Allergy 55:321-337). Upon in-vivo exposure to ozone, pulmonary mast cells have been demonstrated to be activated and to undergo concomitant degranulation, leading to polymorphonuclear cell infiltration into the pulmonary parenchyma (Noviski et al., 1999. J Appl Physiol 86:202-210).

It has recently been reported that antibodies, regardless of their antigen specificity, catalyze via the antibody-mediated water oxidation pathway (as shown via the oxidative cleavage of indigo carmine to produce isatin sulfonic acid with specific isotope labeling; FIG. 2) the conversion of singlet oxygen to an oxidant that exhibits a chemical signature similar to that of ozone (Wentworth, Jr. et al., 2002. Science 298:2195-2199). These findings, and particularly the indications that ozone is also formed by human neutrophils and in inflammatory lesions, have suggested that ozone is generated, not only via the antibody-mediated water oxidation pathway (Wentworth et al., 2000. Proc. Natl. Acad. Sci. U.S.A. 97:10930-10935; Wentworth et al., 2001. Science 293:1806-1811), but also by antibody-coated activated neutrophils. Thus, it has been speculated that since the short lived, but highly toxic, oxidative damage of ozone would be localized to the inflammation site, that ozone could potentially be a proficient effector molecule of the immune response by functioning as a mediator serving to activate immune effector cells such as neutrophils, eosinophils, and lymphocytes, as well as to amplify the inflammatory response, namely by triggering the production of proinflammatory extracellular and intracellular mediators such as NF-kappaB, IL-6, and TNF-alpha (Joos et al., 2000. Allergy 55:321; Cho et al., 2002. Am J Physiol. 280:L537; Wentworth et al., 2002. Science 298:2195-2199).

Taken together, the above described representative examples, as well as many other studies, which delineate the biological signature of ozone, strongly support the notion that ozone may be an important exogenous and endogenous mediator of inflammation in inflammation-associated medical conditions such as asthma, in line with previous suggestions that various reactive oxygen species could act as inflammation mediators and that antioxidant therapy against such reactive oxygen species could be useful in the treatment of asthma (Abraham W M., in: “Asthma,” Barnes, M. et al. Eds., Lippincott-Raven Publishers, Philadelphia 1997, Chapter 47, pp. 627-638).

While conceiving the present invention, the present inventors theorized that inflammation in asthma could involve formation of ozone not only by neutrophils, but also by other white blood cells, and that ozone itself could recruit and activate more leukocytes, which, in turn, would produce more ozone. Hence, according to this hypothesis, a vicious cycle involving leukocyte recruitment and ozone production could account for the persistence of inflammation in asthma, and ozone could thus mediate various asthmatic response phenomena, including mucus secretion, bronchospasm, oedema, and C-fibre activation (FIG. 1).

Various types of compounds, such as olefins, chromone derivatives and xanthine derivatives, due to the electronic nature of their carbon-carbon double bonds, have been shown to be capable of scavenging proinflammatory oxidants such as ozone.

The chemistry of ozone and organic compounds, such as olefins, is well established. During ozonolysis of carbon-carbon double bonds, ozone reacts readily with the double bond in a multistep process that involves initial cycloaddition to produce a primary ozonide, rearrangement to give a more stable ozonide, and cleavage to produce a carbonyl and a carbonyl oxide (Criegee intermediate; Bailey, P. S., Ozonation, in: “Organic Chemistry,” Academic Press: New York, 1978, Vol. 1; ibid 1982; Vol. 2). As is shown in FIG. 3, the reaction proceeds via a formal 1,3-dipolar addition, which is the result of either a concerted addition or a stepwise mechanism, to form the initial 1,2,3-trioxolane product (FIG. 3, A), which thereafter rearranged to form the ozonide (FIG. 3, B). The latter undergoes several additional steps to form the final carbonyl products. It is known that more highly substituted alkenes are more reactive in the ozonolysis reaction; S. M. Japar et al. J. Phys. Chem. 1974, 78, 2318). The fate of the latter depends very much on the reaction conditions and medium. The electrophilic nature of ozone is manifested by the fact that more substituted alkenes, and particularly alkenes with electron-donating substituents, are more reactive ozonolysis substrates, as reflected by their bimolecular rate constants in the gas-phase ozonolysis reaction (FIGS. 2 and 4a-c; Finlayson et al., 1974. J. Am. Chem. Soc. 96:5356; Japar, S. M. et al., 1974. Phys. Chem. 78:2318). Even double bonds that bear both electron-donating and electron-withdrawing groups are highly reactive towards ozone. For example, indigo carmine, which contains a tetra-substituted double bond with two electron-withdrawing groups and two electron-donating groups, serves as a sensitive probe for quantitative detection of ozone in aqueous media via an ozonolysis reaction generating isatin sulfonic acid (FIG. 2; Takeuchi, K. and Takeuchi, I., 1989. Anal. Chem. 61:619; Takeuchi et al., 1990. Anal. Chim. Acta 230:183; Vaya, et al., 2003. Phytochemistry 62:89-99).

Examples of olefins which are capable of scavenging oxidants, such as ozone, by virtue of containing at least one double carbon-carbon bond include volatile monoterpenes (FIG. 4b), such as limonene (Japar et al., 1974. J. Phys. Chem. 78:2318), isoprene (Zhang, D. and Zhang, R. J., 2002. Am. Chem. Soc. 124:2692-2703; Stokes et al., 1998. J. Exp. Bot. 49:115-23), ethylene, and volatile sesquiterpenes (FIG. 4c). Isoprene is one of the most abundant hydrocarbons naturally emitted by the terrestrial biosphere (Kesselmeier and Staudt, 1999. J. Atmos. Chemistry 33:23-88), especially from mosses, ferns, and trees, and the hydrocarbon flux to the atmosphere in the form of isoprene, which is roughly equal to the flux of methane, has a large effect on the oxidizing potential of the atmosphere (Sharkey and Yeh, 1999. Rev. Plant Physiol. Plant Mol. Biol. 52:407-436). Several hypotheses have been proposed to explain the massive emission of isoprene by the biosphere, one of which suggests that isoprene serves as an antioxidant in leaves because this diene reacts rapidly with ozone (Stokes et al., 1998. J. Exp. Bot. 49:115-23). Ethylene, which is a plant ripening and senescence hormone (Lelièvre, A. et al., 1997. Physiol Plant 100:727-739; Jiang and Fu, 2000. Plant Growth Regul. 30:193-200), although less reactive towards ozone than the alkyl-substituted olefins, is also emitted by the biosphere in large quantities. Climacteric fruits have a substantial ethylene production phase during their maturation and ripening (Biale, J. B. and Young, R. E., in: Friend, J. and Rhodes, M. J. C., eds, “Recent Advances in the Biochemistry of Fruits and Vegetables,” Academic Press, London, 1981, pp. 1-39).

Compounds such as volatile olefins, by virtue of being of low molecular weight and lipophilic, have the capacity to saturate pulmonary cell membranes when inhaled. Thus, since volatile olefins, such as the unsaturated terpenes, are much more abundant in rural areas than in the urban environment, it seems likely that relatively high concentration of these lipophilic, membrane soluble olefins, which can act as ozone scavengers, saturate the pulmonary membranes of rural inhabitants, providing them with natural protection against either exogenous or endogenous ozone. It is remarkable that although rural environments are rich in various allergens that can trigger asthma, the prevalence of asthma in rural population is remarkably low in comparison with the urban incidence (Filipiak, B. et al., 2001. Clin Exp Allergy 12:1829-38). Thus, the present inventors hypothesize that the notable worldwide increase in asthma frequency in the last two decades is a result of increased urbanization, which is typically associated with increased industrial air pollution, and more efficiently insulated, air-conditioned living conditions. As such, the present inventors hypothesize that such observations support the hypothesis that ozone scavengers could be used for treating asthma.

As described hereinabove, chromone derivatives, due to the electronic nature of their carbon-carbon double bonds, have been shown to be capable of scavenging proinflammatory oxidants such as ozone. Chromones are ubiquitously found in many naturally occurring phytochemicals, and particularly in flavonoids and isoflavonoids (Forkmann, G. and Heller, W., in: “Comprehensive Natural Products Chemistry,” Barton, D. et al. Eds., Elsevier, UK, 1999. Chapter 1.26, pp. 713-748; Dixon, R. A. ibid Chapter 1.28, pp. 773-823), which possess antioxidant activity (Pietta, P.-G. J., 2000. Nat. Prod. 63:1035-1042; Whiting, D. A., 2001. Nat. Prod. Rep. 18:583-606; Vaya, J. et al., 2003. Phytochemistry 62:89-99). Khellin (FIG. 4d; Atta et al., 1993. Wamhoff, Praktische Chemie/Chemiker-Zeitung 335:225), which is extracted from the seeds of Ammi visnaga (khella), as well as other naturally occurring ozone scavenging chromones are ubiquitously found in plants that have been traditionally used in folklore medicine for the treatment of asthma (FIG. 4d; Whiting, 2001. Nat. Prod. Rep. 18:583-606). Decoctions of khella seeds have been used for centuries as a smooth muscle relaxant, an effect beneficial for alleviating asthmatic bronchoconstriction, and as a diuretic, particularly for the relief of ureteric colic. Khellin, was the first chromone to be used in a pure form in clinical practice for allergy treatment (Edwards, A. M. and Howell, J. B. L., 2000. Clinical & Experimental Allergy 30:756). In 1947, intramuscularly administered khellin was reported to provide complete and prolonged relief in bronchial asthma (Anre, G. V. et al., 1947. Lancet i:557). That report stimulated an effort at Benger's Research Laboratories, UK, to synthesize and develop soluble forms of khellin that could be administered either orally or by inhalation for the treatment of asthma. The effort, which was later continued by Dr. Roger E. C. Altounyan (Edwards, A. M. and Howell, J. B. L., 2000. Clinical & Experimental Allergy 30:756), eventually resulted in the launch of the allergy drugs cromolyn sodium (Intal®) in 1968, and of nedocromil sodium (Tilade®), several years later. Cromolyn sodium and nedocromil sodium are drugs which block bronchospasm and inflammation. The chemical structures of cromolyn sodium and nedocromil sodium are shown in FIG. 4e. The electronic nature of the double bonds in both cromolyn sodium and nedocromil sodium, which are highly reactive towards ozone, is similar to the double bond in indigo carmine (FIG. 2). Although cromolyn sodium and nedocromil sodium are structurally quite different, they share many similar anti-asthma therapeutic/pharmacological effects. Although both of these compounds have been used in the clinic for five decades, their mechanism of action remains unclear and neither a relevant receptor nor an endogenous ligand for these compounds has been found. They have been traditionally classified as “mast cell stabilizers” as it is believed that their primary mode of action is to inhibit the release of inflammatory mediators, such as histamine and prostanoids, from mast cells. This classification, however, is an oversimplification of their multiple pharmacological activities (Bernstein et al., in: “Asthma,” Barnes et al., Eds., Lippincott-Raven Publishers, Philadelphia 1997, Chapter 47, pp. 627-638 and Chapter 113, pp. 1647-1665). In fact, cromolyn sodium is known to inhibit almost any component in the inflammatory cascade of events, including IgE-mediated release of primary inflammatory mediators, such as histamine, prostacyclins, leukotrienes and interleukins, by mast cells and macrophages (Kimata, H. et al., 1991. Clin exp Immunol. 84:395; Loh, R. K. S. et al., 1994. J. Exp. Med. 1994, 180:663; Jabara, H. H. and Geha, R. S. J. 1.994. Exp. Med. 180:663). Cromolyn sodium also inhibits accumulation and activation of eosinophils, neutrophils and lymphocytes, decreases responsiveness of these cells to cytokines and chemotaxins such as platelet activating factor (PAF), and inhibits the activation of macrophages, monocytes and platelets by inflammatory mediators. Cromolyn sodium also inhibits activation of sensory C-fibres by inhibiting the release of tachykinins, such as substance P, and/or by acting as a tachykinin receptor antagonist in vascular endothelial cells (Edwards and Howell, 2000. Clin. Exp. Allergy 30:756). Cromolyn sodium, however, is not a bronchodilator like beta agonists.

Most of the naturally occurring chromones, which have been recognized by folklore medicine as anti-asthmatic agents (FIG. 4d), contain an electron-rich double bond that can scavenge ozone even more efficiently than either nedocromil sodium or nedocromil sodium. For example, khellin contains two reactive unsaturated systems (a trisubstituted double bond with one electron donating group and one electron withdrawing group), both of which undergo rapid ozonolysis upon exposure to ozone (Atta et al., 1993. Praktische Chemie/Chemiker-Zeitung 335:225). Also, the unsaturated system in quercetin (FIG. 4d), found in the bark of the North American black oak, Quercus kelloggii, by virtue of having a tetra-substituted double bond with three electron donating groups and one electron withdrawing group, is even more reactive towards ozone than khellin. Other chromones are found in large quantities in the extracts of other plants that have been used to treat asthma, including Pimpinella anisum (anise), Glycyrrhiza glabra (Gan Cao plant, licorice), Verbascum thapsus (mullein) and others.

A number of xanthine derivatives, including theophylline, enprofylline, 3-isobutyl-1-methylxanthine (IBMX), pentoxifylline, lisofylline, caffeine and uric acid (FIG. 4f) have been used in the clinic to treat asthma, although their mechanism of action is as yet unclear. Chemical and medical studies have highlighted the general antioxidant properties of xanthine derivatives such as pentoxifylline, lisofylline, enprofylline, 1,7-dimethyl enprofylline and their 8-oxo derivatives (Bhat and Madyastha, 2001. Biochem. Biophys. Res. Commun. 288:1212-7; Dent et al., 1994. Am. J. Respir. Cell Mol. Biol. 10:565). Since the central double bond in these compounds is highly electron-rich, it is likely that the pharmacological effect of xanthine derivatives involves, at least partially, ozone-scavenging capability. As these compounds possess an electron-rich double bond that bears three-electron donating groups and one electron-withdrawing group, all xanthines are quite efficient ozone scavengers.

It has been proposed that theophylline (marketed as an asthma drug under various brand names, including Aerolate III®, Aerolate JR®, Aerolate SR®, Aquaphyllin®, Asmalix®, Elixomin®, Elixophyllin®, Quibron®-T, Quibron®-T/SR, Respbid®, Slo-bid™, Slo-Phyllin®, Sustaire®, Theo-24®, Theobid®, Theochron®, Theoclear-80®, Theoclear® L.A., Theo-Dur®, Theolair™, Theo-Sav®, Theospan®-SR, Theostat-80®, Theovent®, Theo-X®, T-Phyl®, Uni-Dur®, and Uniphyl®), at fairly high concentrations, inhibits the production of various reactive oxygen species produced by human alveolar macrophages (Dent et al., 1994. Am. J. Respir. Cell Mol. Biol. 10:565) and that enprofylline retains bronchodilator potency greater than that of theophylline while being a poor antagonist of most classes of adenosine receptors (T. W. Rail, in: The pharmacological basis of therapeutics, 8th ed. Goodman and Gilman (Eds.), 1990, pp. 618-637; Undem and Lichtenstein, in: “The Pharmacological Basis of Therapeutics,” Hardman et al, Eds., 2001, pp. 733-754).

There are strong indications that uric acid, which has a carbon-carbon double bond similar to that of theophylline and which acts as a natural antioxidant in mammals, can combat oxidative stress that is associated with inflammation in various neurological diseases, including multiple sclerosis (Scott, G. S. et al., 2002. Proc. Natl. Acad. Sci. U.S.A. 99:16303-8). It has been reported that serum uric acid levels are closely associated with asthmatic conditions (Ernst et al., 1983. Antimicrob. Agents and Chemother. 24:609), and that persistent hyperuricemia protects against or blunts the manifestation of some symptoms of rheumatoid inflammation (Agudelo, C. A. et al., 1984. Arthritis Rheum. 27:443), and that in humans there is a negative correlation between gout and rheumatoid arthritis. The reason for this mutual exclusion could not be explained yet but a proposal was made that persistent hyperuricemia may protect against or decrease the expression of rheumatoid inflammation (Agudelo, C. A. et al., 1984. Arthritis Rheum. 27:443). It has been observed that serum uric acid levels are increased by theophylline (Morita et al., 1984. J. Allergy Clin. Immun. 74:707). Similarly, it was found that adjuvant arthritis in rats is inhibited by an oxonate diet, which increases uricemia. Remarkably, such inhibition was found to be proportional to blood uric acid level and not to oxonate concentration in the diet (Lussier, A. et al., 1978. Experientia 34:995-6; Chang, Y. H. and Bluestone R., 1981. J Pharmacol Exp Ther. 219:731-4). The ability of uric acid to scavenge ozone was additionally demonstrated by the substantial depletion of uric acid in murine stratum corneum upon acute environmental exposure to ozone (Weber, S. U. et al., 1999. J. Invest. Dermat. 113:1128).

In light of the above described possible causative link between oxidants, such as ozone, and inflammatory processes in inflammation-associated medical conditions, such as asthma, a potentially optimal strategy for prophylaxis and/or treatment of such medical conditions, would be via optimal inhibition of proinflammatory activity by oxidants such as ozone.

Several prior art approaches have been employed or suggested in order to treat inflammation-associated medical conditions by inhibition of proinflammatory activity of oxidants such as ozone.

One approach has suggested inhibiting induction of inflammation by reactive oxygen species, such as superoxide ion, hydrogen peroxide, hydroxyl radical, and hypohalous acids (Abraham, W. M., in: “Asthma,” Barnes et al. Eds., Lippincott-Raven Publishers, Philadelphia 1997, Chapter 47, pp. 627-638).

Another approach has proposed inhibiting induction of inflammation by lipid oxidation products obtained during ozonolysis of unsaturated fatty acids due to the latter causing the release of known proinflammatory mediators, including platelet activation factor (PAF), IL-6, and IL-8 (Pryor et al., 1995. Free Radical Biology and Medicine 19:935-941; Kafoury et al., 1999. Am. J. Respir. Crit. Care Med. 160:1934-1942).

A further approach has suggested or attempted utilizing for asthma prophylaxis or treatment oxidant scavenging chromone derivatives such as khellin or quercetin.

A further approach has suggested or attempted utilizing for asthma prophylaxis or treatment plants, such as anise, licorice, or mullein, or extracts thereof, which include oxidant scavenging chromone derivatives.

A further approach involves using chromone derivative compounds, such as cromolyn sodium or nedocromil sodium for asthma prophylaxis or treatment.

Yet a further approach has attempted utilizing oxidant scavenging xanthine derivatives, such as theophylline, for asthma prophylaxis or treatment.

Still a further approach has attempted utilizing plant extracts containing oxidant scavenging volatile monoterpenes or sesquiterpenes for asthma prophylaxis or treatment.

All of the aforementioned approaches, however, suffer from significant disadvantages.

Nedocromil sodium and cromolyn sodium are usually effective only for asthma that is associated with allergens or exercise and then, typically, only for juvenile asthmatics.

Theophylline has the disadvantages of being a weak bronchodilator with a narrow therapeutic margin, of requiring blood level monitoring to avoid toxicity, and of having a propensity for undesirable drug interactions, for example, its competition for hepatic cytochrome P450 drug-metabolizing enzymes alters plasma levels of several important drugs metabolized by that same system.

Importantly, no prior art approach has suggested the use of isolated terpenes for preventing pathogenic induction of inflammation by proinflammatory oxidants such as ozone in inflammation-associated medical conditions such as asthma.

Critically, no prior art approach has shown optimal efficacy in prophylaxis and/or treatment of inflammation-associated medical conditions, such as asthma, by inhibiting proinflammatory activity by oxidants, such as ozone.

Thus, all prior art approaches have failed to provide an adequate solution for treating inflammation-associated medical conditions such as asthma.

There is thus a widely recognized need for, and it would be highly advantageous to have, a method for treating and/or preventing inflammation-associated medical conditions, such as asthma, devoid of the above limitations.

### SUMMARY OF THE INVENTION

According to one aspect of the present invention there is provided a method of treating a medical condition associated with inflammation in a subject in need thereof, the method comprising administering to the subject a therapeutically effective amount of at least one compound capable of inhibiting an activity and/or a formation of an oxidant associated with the inflammation, with the proviso that when the medical condition is asthma the at least one compound does not include any one Of theophylline, enprofylline, pentoxifylline, lisofylline, 3-isobutyl-1-methylxanthine, caffeine, uric acid, khellin, cromolyn sodium, quercetin and nedocromil sodium.

According to further features in preferred embodiments of the invention described below, the administering is effected via inhalation of the compound.

According to still further features in the described preferred embodiments, the administering is effected at least once daily for a time period that ranges between about 2 hours and about 140 days.

According to still further features in the described preferred embodiments, the administering is effected at least once daily for a time period that ranges between about 8 hours and about 28 days.

According to still further features in the described preferred embodiments, the administering is effected at least once daily for a time period that ranges between about 20 hours and about 14 days.

According to still further features in the described preferred embodiments, the administering is effected substantially continuously during the time-period.

According to still further features in the described preferred embodiments, the therapeutically effective amount is selected such that a concentration of the at least one compound at a site of the inflammation ranges between about 10 ppb and about 1,250 ppm.

According to still further features in the described preferred embodiments, the at least one compound forms a part of a pharmaceutical composition.

According to still further features in the described preferred embodiments, the pharmaceutical composition comprises a pharmaceutically acceptable carrier.

According to yet another aspect of the present invention there is provided a pharmaceutical composition identified for use in the treatment of a medical condition associated with inflammation, comprising, as an active ingredient, at least one compound capable of inhibiting an activity and/or formation of an oxidant associated with the inflammation, and a pharmaceutically acceptable carrier, with the proviso that when the medical condition is asthma the at least one compound does not include any one of theophylline, enprofylline, pentoxifylline, lisofylline, 3-isobutyl-1-methylxanthine, caffeine, uric acid, khellin, cromolyn sodium and nedocromil sodium.

According to further features in preferred embodiments of the invention described below, the pharmaceutical composition is packaged in a packaging material and identified in print, in or on the packaging material, for use in the treatment of the medical condition.

According to still further features in the described preferred embodiments, a dose-unit of the pharmaceutical composition is selected so as to achieve at a site of the inflammation a concentration of the at least one compound that ranges between about 10 ppb and about 1,250 ppm.

According to still further features in the described preferred embodiments, the pharmaceutically acceptable carrier adapts the composition for administration by a route selected from group consisting of intranasal, transdermal, intradermal, oral, buccal, parenteral, topical, rectal and inhalation route.

According to still further features in the described preferred embodiments, the pharmaceutical composition has a form selected from the group consisting of a solution, a suspension, an emulsion, a gel, a foam, a spray, an aerosol and a skin pad.

According to still further features in the described preferred embodiments, the pharmaceutical composition further comprises a formulating agent selected from the group consisting of a propellant, a suspending agent, a stabilizing agent and a dispersing agent.

According to still another aspect of the present invention there is provided an inhalation device for use in the treatment of a medical condition associated with inflammation, comprising at least one compound capable of inhibiting an activity and/or formation of an oxidant associated with the inflammation and a respiratory delivery system, with the proviso that when the medical condition is asthma the at least one compound does not include any one of theophylline, enprofylline, pentoxifylline, lisofylline, 3-isobutyl-1-methylxanthine, caffeine, uric acid, khellin, quercetin, cromolyn sodium and nedocromil sodium.

According to further features in preferred embodiments of the invention described below, the respiratory delivery system is selected from the group consisting of a nebulizer inhaler, a dry powder inhaler, and a metered dose inhaler.

According to still further features in the described preferred embodiments, the respiratory delivery system is configured for delivering the at least compound in a form of a spray or an aerosol.

According to still further features in the described preferred embodiments, the respiratory delivery system is selected from the group consisting of an oil warmer, a vaporizer and an atomizer.

According to still further features in the described preferred embodiments, the respiratory delivery system is configured for oral and/or nasal delivery of the at least one compound.

According to still further features in the described preferred embodiments, the respiratory delivery system is configured for achieving, at a site of the inflammation, a concentration of the at least one compound that ranges between about 10 ppb and about 1,250 ppm.

According to still further features in the described preferred embodiments, the concentration ranges between about 12 ppm and about 1,250 ppm.

According to still further features in the described preferred embodiments, the concentration ranges between about 8 ppm and about 800 ppm.

According to still further features in the described preferred embodiments, the concentration is about 125 ppm.

According to still further features in the described preferred embodiments, the concentration is about 80 ppm.

According to still further features in the described preferred embodiments, the inhibiting is by a stoichiometric reaction of the at least one compound and the oxidant.

According to still further features in the described preferred embodiments, the inhibiting is by a catalytic reaction of the at least one compound and the oxidant.

According to still further features in the described preferred embodiments, the at least one compound is capable of inhibiting a biochemical pathway that produces the oxidant.

According to still further features in the described preferred embodiments, the oxidant is selected from the group consisting of a reactive oxygen species, ozone, hydrogen peroxide, a superoxide ion, a superoxide radical, a hydroxyl radical and a hypohalous acid.

According to still further features in the described preferred embodiments, the oxidant is ozone.

According to still further features in the described preferred embodiments, the at least one compound is capable of scavenging the ozone.

According to still further features in the described preferred embodiments, the scavenging is by a stoichiometric reaction of the at least one compound and the ozone.

According to still further features in the described preferred embodiments, the scavenging is by a catalytic reaction of the at least one compound and the ozone.

According to still further features in the described preferred embodiments, the at least one compound is capable of inhibiting a biochemical pathway that produces the ozone.

According to still further features in the described preferred embodiments, the at least one compound is substantially lipophilic and/or hydrophobic.

According to still further features in the described preferred embodiments, the at least one compound is substantially volatile.

According to still further features in the described preferred embodiments, the at least one compound is selected from the group consisting of an alkene, an α,β-unsaturated carbonyl, a terpene, a xanthine, a chromone, an unsaturated fatty acid, an indigoid, an organic conductor and any derivative or analog thereof.

According to still further features in the described preferred embodiments, the alkene has between 2 and 15 carbon atoms.

According to still further features in the described preferred embodiments, the alkene is selected from the group consisting of ethylene, propylene, 1-butene, trans-2-butene, cis-2-butene, 2-methyl-2-butene, isoprene, butadiene, 2,3-dimethyl-2-butene, cyclohexene, cyclohexadiene and cyclopentene.

According to still further features in the described preferred embodiments, the terpene is selected from the group consisting of a monoterpene and a sesquiterpene.

According to still further features in the described preferred embodiments, the monoterpene is selected from the group consisting of citronellol, geraniol, nerol, linalool, citral, carvone, pulegone, limonene, myrcene, α-terpinen, γ-terpinene, terpinolene, careen, terpinol, α-terpinol, α-thujene, lavandulol, α-pinene, β-pinene, myrtenol, camphene and rosoxide.

According to still further features in the described preferred embodiments, the sesquiterpene is selected from the group consisting of β-caryophyllene, β-santalene, alantolactone, cyperene, longipinene, nerolidol, (z)-γ-bisabolene, β-elemene, β-eudesmene, γ-cadinene, epi-zonarene, bicyclogemarcene, (z)-γ-bisabulene and γ-himachalene.

According to still further features in the described preferred embodiments, the xanthine or the derivative or analog thereof has a general Formula I:

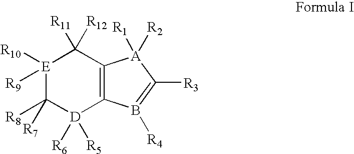

wherein: each of A, D and E is independently selected from the group consisting of carbon, nitrogen, oxygen and sulfur; B is carbon or nitrogen; and each of R₁-R₁₂ is independently selected from the group consisting of hydrogen, lone pair electrons, alkyl, hydroxyalkyl, trihaloalkyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azo, sulfonyl, sulfinyl, sulfonamide, phosphonyl, phosphinyl, phosphonium, ketoester, ketone, carbonyl, thiocarbonyl, ether, thiocarboxy, thioether, thiocarbamate, urea, thiourea, O-carbamyl, N-carbamyl, O-thiocarbamyl, N-thiocarbamyl, C-amido, N-amido, C-carboxy, O-carboxy, guanyl, guanidine and amino, or, alternatively, at least two of R₁-R₄ and/or at least two of R₅-R₁₂ form at least one five- or six-membered aromatic, heteroaromatic, alicyclic or heteroalicyclic ring, or a pharmaceutically acceptable salt thereof.

According to still further features in the described preferred embodiments, each of A, B, D and E is nitrogen.

According to still further features in the described preferred embodiments, each of R₇ and R₁₁ is carbonyl and R₈ and R₁₂ are absent.

According to still further features in the described preferred embodiments, the chromone or the derivative or analog thereof has a general Formula II:

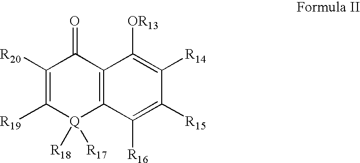

wherein: Q is carbon, oxygen, sulfur or nitrogen; and each of R₁₃-R₂₀ is independently selected from the group consisting of hydrogen, lone pair electrons, alkyl, hydroxyalkyl, trihaloalkyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azo, sulfonyl, sulfinyl, sulfonamide, phosphonyl, phosphinyl, phosphonium, ketone, ketoester, carbonyl, thiocarbonyl, ether, thiocarboxy, thioether, thiocarbamate, urea, thiourea, O-carbamyl, N-carbamyl, O-thiocarbamyl, N-thiocarbamyl, C-amido, N-amido, C-carboxy, O-carboxy, guanyl, guanidine and amino, or, alternatively, at least two of R₁₃-R₁₆ and/or at least two of R₁₇-R₂₀ form at least one five- or six-membered aromatic, heteroaromatic, alicyclic or heteroalicyclic ring, or a pharmaceutically acceptable salt thereof.

According to still further features in the described preferred embodiments, Q is oxygen.

According to still further features in the described preferred embodiments, R₁₆ is selected from the group consisting of hydroxy, alkoxy, aryloxy, thiohydroxy, thioalkoxy, and thioaryloxy. According to still further features in the described preferred embodiments, the unsaturated fatty acid or the derivative or analog thereof are selected from the group consisting of linoleic acid, linolenic acid, oleic acid, palmitoleic acid, arachidonic acid and any derivative, analog or a pharmaceutically acceptable salt thereof.

According to still further features in the described preferred embodiments, the indigoid or the derivative or analog thereof has the general Formula III:

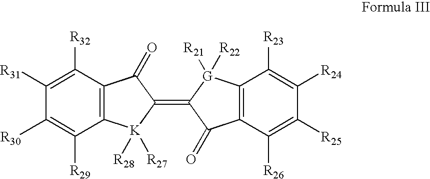

wherein: each of G and K is independently selected from the group consisting of carbon, oxygen, sulfur or nitrogen; and each of R₂₁-R₃₂ is independently selected from the group consisting of hydrogen, lone pair electrons, alkyl, hydroxyalkyl, trihaloalkyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azo, sulfonyl, sulfinyl, sulfonamide, phosphonyl, phosphinyl, phosphonium, ketone, ketoester, carbonyl, thiocarbonyl, ether, thiocarboxy, thioether, thiocarbamate, urea, thiourea, O-carbamyl, N-carbamyl, O-thiocarbamyl, N-thiocarbamyl, C-amido, N-amido, C-carboxy, O-carboxy, guanyl, guanidine and amino, or, alternatively, at least two of R₂₁-R₂₆ and/or at least two of R₂₇-R₃₂ form at least one five- or six-membered aromatic, heteroaromatic, alicyclic or heteroalicyclic ring, or a pharmaceutically acceptable salt thereof.

According to still further features in the described preferred embodiments, each of G and K is nitrogen.

According to still further features in the described preferred embodiments, at least one of R₂₃, R₂₆, R₂₉ and R₃₂ is selected from the group consisting of hydroxy, alkoxy, aryloxy, thiohydroxy, thioalkoxy, and thioaryloxy.

According to still further features in the described preferred embodiments, the α,β-unsaturated carbonyl has a general Formula IV:

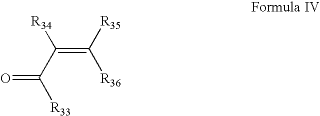

wherein each of R₃₃-R₃₆ is independently selected from the group consisting of hydrogen, alkyl, alkenyl, hydroxyalkyl, trihaloalkyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azo, sulfonyl, sulfinyl, sulfonamide, phosphonyl, phosphinyl, phosphonium, ketone, ketoester, carbonyl, thiocarbonyl, ether, thiocarboxy, thioether, thiocarbamate, urea, thiourea, O-carbamyl, N-carbamyl, O-thiocarbamyl, N-thiocarbamyl, C-amido, N-amido, C-carboxy, O-carboxy, guanyl, guanidine and amino, or, alternatively, at least two of R₃₃-R₃₆ form at least one five- or six-membered alicyclic or heteroalicyclic ring, or a pharmaceutically acceptable salt thereof.

According to still further features in the described preferred embodiments, each R₃₃ is selected from the group consisting of hydroxy, alkoxy, aryloxy, thiohydroxy, thioalkoxy, and thioaryloxy.

According to still further features in the described preferred embodiments, the organic conductor has a general Formula V or VI:

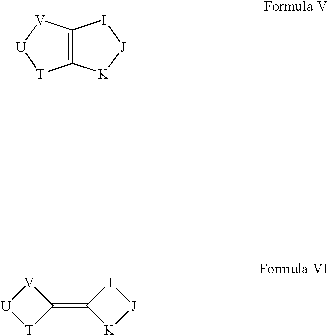

wherein: each of I, K, T and V is independently selected from the group consisting of CR₃₇R₃₈, NR₃₉, O and S; each of J and K is independently selected from the group consisting of CR₄₀R₄₁, R₄₂R₄₃C—CR₄₄R₄₅, R₄₆R₄₇C—C(R₄₈R₄₉)—C(R₅₀R₅₁)—CR₅₂R₅₃ and R₅₄C═CR₅₅; and each of R₃₇-R₅₅ is independently selected from the group consisting of hydrogen, alkyl, alkenyl, hydroxyalkyl, trihaloalkyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azo, sulfonyl, sulfinyl, sulfonamide, phosphonyl, phosphinyl, phosphonium, ketone, ketoester, carbonyl, thiocarbonyl, ether, thiocarboxy, thioether, thiocarbamate, urea, thiourea, O-carbamyl, N-carbamyl, O-thiocarbamyl, N-thiocarbamyl, C-amido, N-amido, C-carboxy, O-carboxy, guanyl, guanidine and amino, or, alternatively, at least two of R₃₇-R₅₅ form at least one five- or six-membered aromatic, heteroaromatic, alicyclic or heteroalicyclic ring, or a pharmaceutically acceptable salt thereof.

According to still further features in the described preferred embodiments, the at least one compound has a general Formula VII:

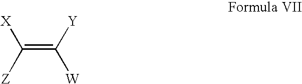

wherein each of X, Y, Z and W is independently selected from the group consisting of hydrogen, alkyl, alkenyl, hydroxyalkyl, trihaloalkyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azo, sulfonyl, sulfinyl, sulfonamide, phosphonyl, phosphinyl, phosphonium, ketone, ketoester, carbonyl, thiocarbonyl, ether, thiocarboxy, thioether, thiocarbamate, urea, thiourea, O-carbamyl, N-carbamyl, O-thiocarbamyl, N-thiocarbamyl, C-amido, N-amido, C-carboxy, O-carboxy, guanyl, guanidine and amino, or, alternatively, at least two of X, Y, Z and W form at least one five- or sik-membered aromatic, heteroaromatic, alicyclic or heteroalicyclic ring, or a pharmaceutically acceptable salt thereof.

According to still further features in the described preferred embodiments, each of X, Y, Z and W is independently hydrogen or alkyl, or alternatively, two of X, Y, Z and W form a five- or six-membered alicyclic ring.

According to still further features in the described preferred embodiments, at least two of X, Y, Z and W are each independently alkyl or cycloalkyl.

According to still further features in the described preferred embodiments, at least two of X, Y, Z and W is an alkyl or alkenyl having at least 6 carbon atoms and at least one of the alkyl or alkenyl terminates with a C(═O)-L group or a pharmaceutically acceptable salt thereof, L is selected from the group consisting of hydrogen, alkyl, cycloalkyl, aryl, hydroxy, alkoxy, aryloxy and amino.

According to still further features in the described preferred embodiments, at least one of X, Y, Z and W is C-carboxy or a pharmaceutically acceptable salt thereof.

According to still further features in the described preferred embodiments, at least two of X, Y, Z and W form at least one five- or six-membered aromatic, heteroaromatic, alicyclic and/or heteroalicyclic ring.

According to still further features in the described preferred embodiments, the at least one compound has a general Formula I, II, III, V or VI, as described hereinabove.

According to still further features in the described preferred embodiments, at least one of X, Y, Z and W is an electron-donating group.

According to still further features in the described preferred embodiments, the at least one compound is a metalloporphyrin or any derivative, analog or pharmaceutically acceptable salt thereof.

According to still further features in the described preferred embodiments, the medical condition is selected from the group consisting of an inflammatory disease, an idiopathic inflammatory disease, a chronic inflammatory disease, an acute inflammatory disease, an allergic disease, an autoimmune disease, an infectious disease, an inflammatory malignant disease, an inflammatory transplantation-related disease, an inflammatory degenerative disease, an inflammatory injury, a disease associated with a hypersensitivity, an inflammatory cardiovascular disease, an inflammatory glandular disease, an inflammatory gastrointestinal disease, an inflammatory cutaneous disease, an inflammatory hepatic disease, an inflammatory neurological disease, an inflammatory musculo-skeletal disease, an inflammatory renal disease, an inflammatory reproductive disease, an inflammatory systemic disease, an inflammatory connective tissue disease, an inflammatory tumor, necrosis, an inflammatory implant-related disease and an inflammatory pulmonary disease.

According to still further features in the described preferred embodiments, the allergic disease is selected from the group consisting of asthma, hives, urticaria, a pollen allergy, a dust mite allergy, a venom allergy, a cosmetics allergy, a latex allergy, a chemical allergy, a drug allergy, an insect bite allergy, an animal dander allergy, a stinging plant allergy, a poison ivy allergy, anaphylactic shock, anaphylaxis, and a food allergy.

According to still further features in the described preferred embodiments, the hypersensitivity is selected from the group consisting of Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity, delayed type hypersensitivity, helper T lymphocyte mediated hypersensitivity, cytotoxic T lymphocyte mediated hypersensitivity, TH1 lymphocyte mediated hypersensitivity, and TH2 lymphocyte mediated hypersensitivity.

According to still further features in the described preferred embodiments, the inflammatory cardiovascular disease is selected from the group consisting of occlusive disease, atherosclerosis, myocardial infarction, thrombosis, Wegener's granulomatosis, Takayasu's arteritis, Kawasaki syndrome, anti-factor VIII autoimmune disease, necrotizing small vessel vasculitis, microscopic polyangiitis, Churg and Strauss syndrome, pauci-immune focal necrotizing glomerulonephritis, crescentic glomerulonephritis, antiphospholipid syndrome, antibody induced heart failure, thrombocytopenic purpura, autoimmune hemolytic anemia, cardiac autoimmunity, Chagas' disease, and anti-helper T lymphocyte autoimmunity.

According to still further features in the described preferred embodiments, the inflammatory glandular disease is selected from the group consisting of pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome.

According to still further features in the described preferred embodiments, the inflammatory gastrointestinal disease is selected from the group consisting of colitis, ileitis, Crohn's disease, chronic inflammatory intestinal disease, inflammatory bowel syndrome, chronic inflammatory bowel disease, celiac disease, an ulcer, a skin ulcer, a bed sore, a gastric ulcer, a peptic ulcer, a buccal ulcer, a nasopharyngeal ulcer, an esophageal ulcer, a duodenal ulcer and a gastrointestinal ulcer.

According to still further features in the described preferred embodiments, the inflammatory cutaneous disease is selected from the group consisting of acne, autoimmune bullous skin disease, pemphigus vulgaris, bullous pemphigoid, pemphigus foliaceus, contact dermatitis and drug eruption.

According to still further features in the described preferred embodiments, the inflammatory hepatic disease is selected from the group consisting of autoimmune hepatitis, hepatic cirrhosis, and biliary cirrhosis.

According to still further features in the described preferred embodiments, the inflammatory neurological disease is selected from the group consisting of multiple sclerosis, Alzheimer's disease, Parkinson's disease, myasthenia gravis, motor is neuropathy, Guillain-Barre syndrome, autoimmune neuropathy, Lambert-Eaton myasthenic syndrome, paraneoplastic neurological disease, paraneoplastic cerebellar atrophy, non-paraneoplastic stiff man syndrome, progressive cerebellar atrophy, Rasmussen's encephalitis, amyotrophic lateral sclerosis, Sydeham chorea, Gilles de la Tourette syndrome, autoimmune polyendocrinopathy, dysimmune neuropathy, acquired neuromyotonia, arthrogryposis multiplex, optic neuritis, spongiform encephalopathy, migraine, headache, cluster headache, and stiff-man syndrome.

According to still further features in the described preferred embodiments, the inflammatory connective tissue disease is selected from the group consisting of autoimmune myositis, primary Sjogren's syndrome, smooth muscle autoimmune disease, myositis, tendinitis, a ligament inflammation, chondritis, a joint inflammation, a synovial inflammation, carpal tunnel syndrome, arthritis, rheumatoid arthritis, osteoarthritis, ankylosing spondylitis, a skeletal inflammation, an autoimmune ear disease, and an autoimmune disease of the inner ear.

According to still further features in the described preferred embodiments, the inflammatory renal disease is autoimmune interstitial nephritis and/or renal cancer.

According to still further features in the described preferred embodiments, the inflammatory reproductive disease is repeated fetal loss, ovarian cyst, or a menstruation associated disease.

According to still further features in the described preferred embodiments, the inflammatory systemic disease is selected from the group consisting of systemic lupus erythematosus, systemic sclerosis, septic shock, toxic shock syndrome, and cachexia.

According to still further features in the described preferred embodiments, the infectious disease is selected from the group consisting of a chronic infectious disease, a subacute infectious disease, an acute infectious disease, a viral disease, a bacterial disease, a protozoan disease, a parasitic disease, a fungal disease, a mycoplasma disease, gangrene, sepsis, a prion disease, influenza, tuberculosis, malaria, acquired immunodeficiency syndrome, and severe acute respiratory syndrome.

According to still further features in the described preferred embodiments, the inflammatory transplantation-related disease is selected from the group consisting of graft rejection, chronic graft rejection, subacute graft rejection, acute graft rejection hyperacute graft rejection, and graft versus host disease.

According to still further features in the described preferred embodiments, the implant is selected from the group consisting of a prosthetic implant, a breast implant, a silicone implant, a dental implant, a penile implant, a cardiac implant, an artificial joint, a bone fracture repair device, a bone replacement implant, a drug delivery implant, a catheter, a pacemaker, an artificial heart, an artificial heart valve, a drug release implant, an electrode, and a respirator tube.

According to still further features in the described preferred embodiments, the inflammatory tumor is selected from the group consisting of a malignant tumor, a benign tumor, a solid tumor, a metastatic tumor and a non-solid tumor.

According to still further features in the described preferred embodiments, the inflammatory injury is selected from the group consisting of an abrasion, a bruise, a cut, a puncture wound, a laceration, an impact wound, a concussion, a contusion, a thermal burn, frostbite, a chemical burn, a sunburn, a desiccation, a radiation burn, a radioactivity burn, a smoke inhalation, a torn muscle, a pulled muscle, a torn tendon, a pulled tendon, a pulled ligament, a torn ligament, a hyperextension, a torn cartilage, a bone fracture, a pinched nerve and a gunshot wound.

According to still further features in the described preferred embodiments, the inflammatory pulmonary disease is selected from the group consisting of asthma, allergic asthma, emphysema, chronic obstructive pulmonary disease and bronchitis.

According to still further features in the described preferred embodiments, the inflammation is associated with a biological activity selected from the group consisting of cellular histamine synthesis or secretion, cellular leukotriene synthesis or secretion, lymphocytic adhesion at a site of the inflammation, lymphocytic migration to a site of the inflammation, lymphocytic aggregation at a site of the inflammation, granulocytic migration to a site of the inflammation, vascular permeabilization at a site of the inflammation, and antibody production at a site of the inflammation.

According to a further aspect of the present invention there is provided a method of qualifying a presence of an oxidant in a sample, the method comprising: contacting the sample with a medium containing a compound in a mesophase state, the compound being susceptible to a degradation induced by the oxidant; and evaluating a capacity of the sample to induce the degradation of the compound, thereby qualifying the presence of the oxidant in the sample.

According to further features in preferred embodiments of the invention described below, the sample comprises at least one cell.

According to yet a further aspect of the present invention there is provided a method of diagnosing a medical condition associated with inflammation in a subject, the method comprising: contacting at least one cell being derived from the subject and capable of producing an oxidant, with a medium containing a compound in a mesophase state, the compound being susceptible to a degradation induced by the oxidant; and evaluating a capacity of the at least one cell to induce the degradation of the compound, thereby diagnosing the medical condition associated with the inflammation.

According to further features in preferred embodiments of the invention described below, the method of diagnosing the medical condition further comprises isolating the at least one cell from the subject prior to or following the contacting.

According to still further features in the described preferred embodiments, the at least one cell is derived from a site of the inflammation and/or a body fluid of the subject.

According to still a further aspect of the present invention there is provided a method of identifying a candidate compound for treating a medical condition associated with inflammation, the method comprising: exposing a medium containing a compound in a mesophase state and a test compound to an oxidant, the compound in the mesophase state being susceptible to a degradation induced by the oxidant, wherein the medical condition is associated with an activity and/or formation of the oxidant; and evaluating a capacity of the test compound to regulate the degradation, thereby identifying the candidate compound for treatment of the medical condition associated with inflammation.

According to further features in preferred embodiments of the invention described below, the exposing is effected by contacting the medium with at least one cell which is capable of producing the oxidant.

According to still further features in the described preferred embodiments, the evaluating is effected by measuring and/or characterizing a physical property of the medium.

According to still further features in the described preferred embodiments, the physical property is a phase of matter.

According to still further features in the described preferred embodiments, the physical property is an optical birefringence.

According to still further features in the described preferred embodiments, the at least one cell is an immune cell.

According to still further features in the described preferred embodiments, the at least one cell is an activated immune cell.

According to still further features in the described preferred embodiments, the at least one cell is an effector cell.

According to still further features in the described preferred embodiments, the at least one cell is a myeloid cell.

According to still further features in the described preferred embodiments, the at least one cell is a granulocyte.

According to still further features in the described preferred embodiments, the at least one cell is a neutrophil.

According to still further features in the described preferred embodiments, the at least one cell is derived from a cultured cell line.

According to still further features in the described preferred embodiments, the mesophase state is a lyotropic mesophase state and/or a cholesteric mesophase state.

According to still further features in the described preferred embodiments, the compound in the mesophase state is a chromone derivative.

According to still further features in the described preferred embodiments, the chromone derivative is cromolyn sodium.

According to still further features in the described preferred embodiments, the oxidant is selected from the group consisting of a reactive oxygen species, ozone, hydrogen peroxide, a superoxide ion, a superoxide radical, a hydroxyl radical and a hypohalous acid.

According to still further features in the described preferred embodiments, the oxidant is ozone.

The present invention successfully addresses the shortcomings of the presently known configurations by providing an optimal method, composition and inhalation device for treating an inflammation-associated medical condition; and by providing an optimal method of identifying a candidate compound for treating such a medical condition.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 presents a schematic diagram depicting proposed mechanisms involving ozone (O₃) as an inflammatory mediator in asthma.

FIG. 2 presents a scheme depicting the ozonolysis of indigo carmine to yield isatin sulfonic acid.

FIG. 3 presents a scheme depicting the multistep ozonolysis of olefins.

FIG. 4a presents a set of general chemical structures of simple olefins and their relative reactivities towards ozone.

FIG. 4b presents the chemical structures of a set of various volatile monoterpenes found in plants whose extracts have traditionally been used as folklore remedies for asthma.

FIG. 4c presents the chemical structures of a set of various volatile sesquiterpenes found in plants whose extracts have traditionally been used as folklore remedies for asthma.

FIG. 4d presents the chemical structures of a set of representative plant derived chromones used in folklore medicine for asthma treatment.

FIG. 4e presents the chemical structures of the antiasthmatic chromone drugs cromolyn sodium (Intal®) and nedocromil sodium (Tilade®).

FIG. 4f presents the chemical structures of various xanthine derivatives used to treat asthma.

FIG. 5 presents schemes depicting the chemical reactions of fumaric acid derivatives and ketylidene derivatives with ozone.

FIG. 6 is a schematic diagram depicting a metered-dose inhaler (MDI). Arrows indicate direction of fluid flow.

FIG. 7 is a schematic diagram depicting a nebulizer. Arrows indicate direction of fluid flow.

FIG. 8 is a schematic diagram depicting a dry powder inhaler (DPI). Arrows indicate direction of fluid flow.

FIG. 9 is a schematic diagram depicting an oil warmer.

FIGS. 10a-b present vis-à-vis the chemical structures of limonene (FIG. 10a) and eucalyptol (FIG. 10b).

FIG. 11 is a bar graph depicting the enhanced pause (Penh) value, a measure of pulmonary function, in untreated ovalbumin sensitized (asthma), untreated non ovalbumin sensitized (naïve), limonene treated ovalbumin sensitized (limonene), and eucalyptol treated ovalbumin sensitized (eucalyptol) animals subjected to secondary challenge with ovalbumin. Error bars represent standard error.

FIGS. 12a-d are photomicrographs of histological analyses of lung sections depicting pathological changes in the lungs of representative members of the four experimental groups. FIG. 12a depicts a section from an OVA unsensitized, OVA challenged, untreated (naïve) rat showing minimal peribronchiolar inflammatory infiltrate. FIG. 12b depicts a section from an OVA sensitized, OVA challenged, untreated rat exhibiting marked inflammatory infiltrate surrounding the bronchiole in the center and evidence of bronchoconstriction (the mucosa is thrown into folds). FIG. 12c depicts a section from limonene treated rat showing minimal to mild peribronchiolar inflammatory infiltration and no morphologic evidence of bronchoconstriction. FIG. 12d depicts a section from an OVA sensitized, OVA challenged, eucalyptol treated rat exhibiting peribronchiolar inflammation and a lymphoid follicle. The bronchiole in the upper right corner of this slide shows morphologic evidence of bronchoconstriction. Sections were stained with H&E, and photographed at an original magnification of ×20.

FIG. 13 is a histogram depicting the average pathological examination scores of the pathologist's assessment of peribronchiolar inflammation (dark blue bars), perivascular inflammation (purple bars), granulomatous response (white bars) and bronchoconstriction/papillary infolding of the bronchiolar mucosa; (sky blue bars) in untreated ovalbumin sensitized (asthma), untreated non ovalbumin sensitized (naïve), limonene treated ovalbumin sensitized (limonene), and eucalyptol treated ovalbumin sensitized (eucalyptol) animals subjected to repeated ovalbumin inhalation. Error bars represent standard error.

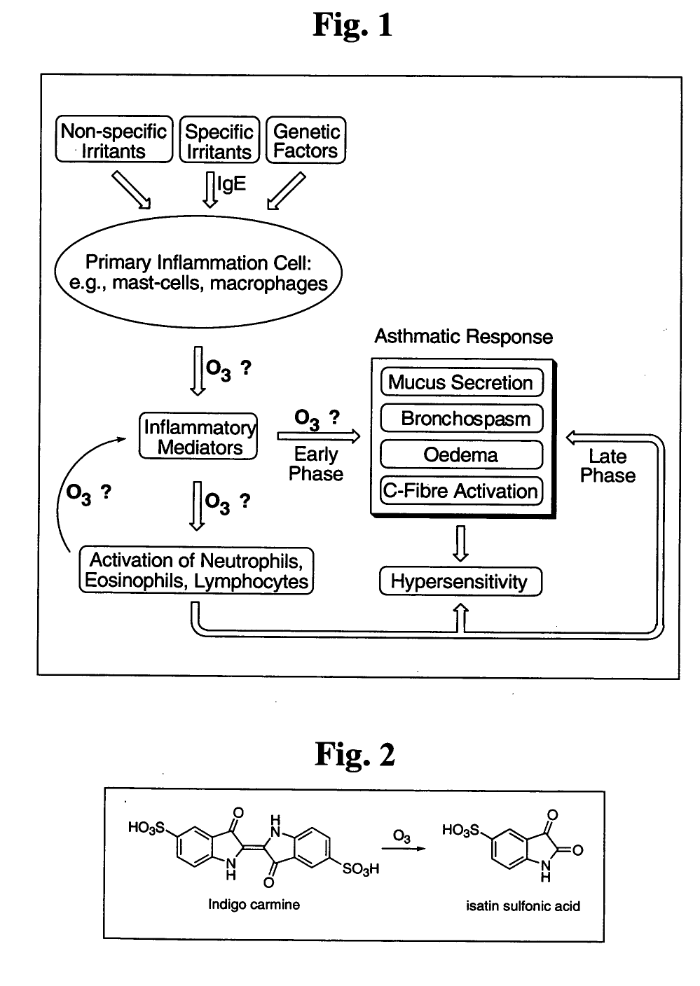

### DESCRIPTION OF THE PREFERRED EMBODIMENTS

The present invention is of methods, pharmaceutical compositions and devices which can be used to treat inflammation-associated medical conditions. Specifically, the present invention employs compounds capable of inhibiting inflammation mediated by oxidants such as ozone for treating inflammation-associated medical conditions, and inhalation devices capable of delivering such compounds to airway tissues. The present invention is further of methods of detecting an oxidant in a sample which methods employing assays based on detection of protection of an oxidation sensitive mesophase from degradation induced by an oxidant, which can be used for diagnosing inflammation-associated medical conditions and for identifying candidate compounds for treating such medical conditions. Specifically, the methods employ assays based on detection of protection of an oxidation sensitive mesophase from degradation induced by a proinflammatory oxidant, such as ozone, and can be used for diagnosing an inflammation-associated medical condition involving inflammation mediated by such an oxidant.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or exemplified in the Examples. The invention is capable of other embodiments or of being practiced or carried out in various ways. Also, it is to be understood that the phraseology and terminology employed herein is for the purpose of description and should not be regarded as limiting.

Inflammation-associated medical conditions, which include numerous allergic, autoimmune, infectious, transplantation-related, malignant, degenerative and injury-related diseases, are highly debilitating and/or lethal diseases for which no optimal therapy exists. While various approaches have been proposed or attempted in the prior art for treating such medical conditions by inhibiting inflammatory processes associated therewith, none of these have resulted in optimal methods of treating such medical conditions.

Thus, all prior art approaches have failed to provide adequate solutions for treating inflammation-associated medical conditions.

While conceiving the present invention, it was hypothesized that proinflammatory oxidants such as ozone in particular may be critical mediators of pathogenic inflammation in inflammation-associated medical conditions such as asthma, and hence that an optimal strategy for treatment/prophylaxis of such medical conditions would be via optimal inhibition of activity/formation of such proinflammatory oxidants.

While reducing the present invention to practice it was unexpectedly uncovered, using a highly realistic in-vivo animal asthma model, and using clinical trials in asthmatic human patients, as described in detail in Examples 1 and 2, respectively, of the Examples section below, that administration of an oxidant scavenging compound, such as d-limonene, could be used to potently and optimally treat inflammation-associated medical conditions, such as asthma, in mammals in general and in humans in particular. These findings clearly demonstrate the linkage and dependency between oxidants and inflammation and thus provide for novel and innovative treatment for asthma and other inflammation-associated medical conditions.

Thus, according to one aspect of the present invention there is provided a method of treating a medical condition associated with inflammation in a subject in need thereof. The method is effected by administering to the subject a therapeutically effective amount of at least one compound capable of inhibiting an activity and/or a formation of an oxidant associated with the inflammation. The method can be used for optimally treating in a subject essentially any inflammation-associated medical condition in which an inflammation is mediated by an oxidant, such as ozone. The method is particularly suitable for treating in a subject an allergic/inflammatory pulmonary disease in which an inflammation is mediated by ozone, such as asthma. While the method is suitable for treating the medical condition in essentially any homeothermic vertebrate, the method is preferably used for treating the medical condition in a mammal, in particular a human.

As used herein, the phrases “inflammatory medical condition,” “inflammation-associated medical condition,” and “medical condition associated with inflammation” are used interchangeably, and refer to any medical condition whose pathogenesis involves inflammation, any medical condition which is accompanied by a manifestation of inflammation, and/or any medical condition of which inflammation is a symptom.

As used herein, the term “method” refers to manners, means, techniques and/or procedures for accomplishing a given task including, but not limited to, those manners, means, techniques and/or procedures either known to, or readily developed from known manners, means, techniques and/or procedures by practitioners of the chemical, pharmacological, biological, biochemical and/or medical arts.

As used herein, the term “treating,” when relating to the medical condition, refers to curing the medical condition, reversing progression of the medical condition and/or a symptom thereof, halting progression of the medical condition and/or a symptom thereof, slowing progression of the medical condition and/or a symptom thereof, alleviating the medical condition and/or a symptom thereof, palliating the medical condition and/or a symptom thereof, preventing onset of the medical condition and/or a symptom thereof, delaying onset of the medical condition and/or a symptom thereof, and/or ameliorating the medical condition and/or a symptom thereof.

As used herein, the phrase “medical condition” refers to any medical disease, disorder, and/or syndrome; and/or to any undesired and/or abnormal physiological, morphological and/or physical state and/or condition.

As used herein, the phrase “activity and/or formation of an oxidant associated with the inflammation” is used interchangeably with the phrase “oxidant activity/formation”, and refers to any activity/formation of an oxidant of the present invention which is associated with initiation, progression, exacerbation, increase, amplification, maintenance and/or prolongation of the inflammation.

As used herein, the term “inhibiting,” when relating to the oxidant activity/formation, refers to preventing, slowing, decreasing, reversing and/or halting the oxidant activity/formation.

Thus, by “inhibiting an activity of an oxidant associated with the inflammation” it is meant herein inhibiting the mediating activity of an oxidant which results in the inflammation, to thereby reduce or prevent the inflammation.

By “inhibiting a formation of an oxidant associated with the inflammation” it is meant herein inhibiting the in-vivo secretion or formation of the oxidant, in the case of a biologically generated oxidant, to thereby reduce or prevent the onset of the inflammation.

The compounds used in the various aspects of the present invention in general, and in this aspect in particular, are therefore selected capable of inhibiting activity/formation of any of various oxidants associated with inflammation (proinflammatory oxidants), depending on the application and purpose.

According to the present invention, and based on the presently known art, the oxidant is preferably ozone and the compounds described herein are selected capable of inhibiting activity/formation of ozone.

However, as described above, it should be appreciated that other oxidants formed in-vivo can mediate inflammation and therefore the compounds described herein may advantageously be selected capable of inhibiting activity/formation of an oxidant such as a reactive oxygen species, hydrogen peroxide, and/or hypohalous acid. Examples of reactive oxygen species include, but are not limited to, a superoxide ion, a superoxide radical, and a hydroxyl radical.

The compounds described herein may be advantageously selected capable of inhibiting the oxidant activity/formation via any of various mechanisms. Preferably, the compounds are selected capable of inhibiting the oxidant activity via scavenging of the oxidant. Alternately, as is described hereinabove and as further detailed hereinbelow, the compounds may be selected capable of inhibiting the formation of the oxidant by inhibiting a biochemical pathway that produces the oxidant (referred to hereinafter as “oxidant-producing biochemical pathway”).

As used herein, the term “scavenging” refers to removal of a substance by means of a chemical or physical reaction therewith. Such chemical reactions may include decomposing the substance by the reaction, changing its chemical structure and properties by this reaction or utilizing the substance by the reaction. Such physical reactions include chelation, absorption, steric masking, and the like.

The chemical reaction described hereinabove can be either stoichiometric or catalytic.

Hence, the scavenging of the oxidant, according to the present invention, can be performed by a stoichiometric reaction of the oxidant and one or more of the compounds described herein, or by a catalytic reaction between the oxidant and one or more of the compounds described herein.

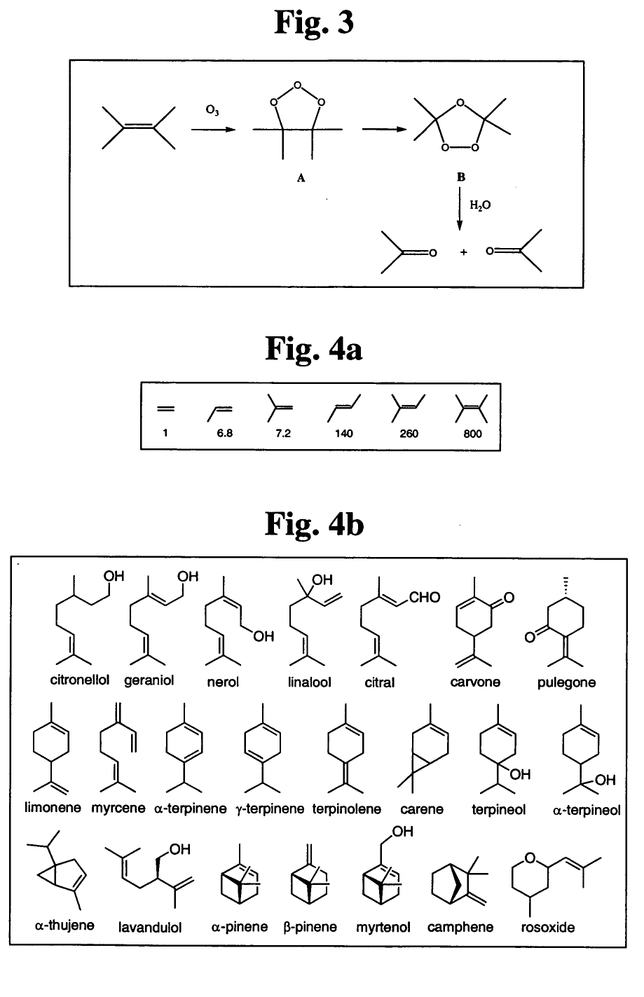

As is discussed in detail hereinabove, electron rich olefins are known to readily react with oxidants such as ozone, to thereby scavenge the oxidant (see, for example, FIG. 3). Such a reaction between olefins and oxidants is typically stoichiometric.

As used herein, the term “olefin” or the phrase “olefin moiety” describes a compound or a moiety that has at least two carbon atoms and at least one carbon-carbon double bond.

Hence, in a preferred embodiment of the present invention, each of the compounds described herein has the general Formula VII:

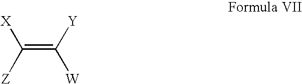

wherein each of X, Y, Z and W is independently selected from the group consisting of hydrogen, alkyl, alkenyl, hydroxyalkyl, trihaloalkyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azo, sulfonyl, sulfinyl, sulfonamide, phosphonyl, phosphinyl, phosphonium, carboxy, ketoester, carbonyl, thiocarbonyl, ether, thiocarboxy, thioether, thiocarbamate, urea, thiourea, O-carbamyl, N-carbamyl, O-thiocarbamyl, N-thiocarbamyl, C-amido, N-amido, C-carboxy, O-carboxy, guanyl, guanidine and amino, or, alternatively, at least two of X, Y, Z and W form at least one five- or six-membered aromatic, heteroaromatic, alicyclic or heteroalicyclic ring, or a pharmaceutically acceptable salt thereof.

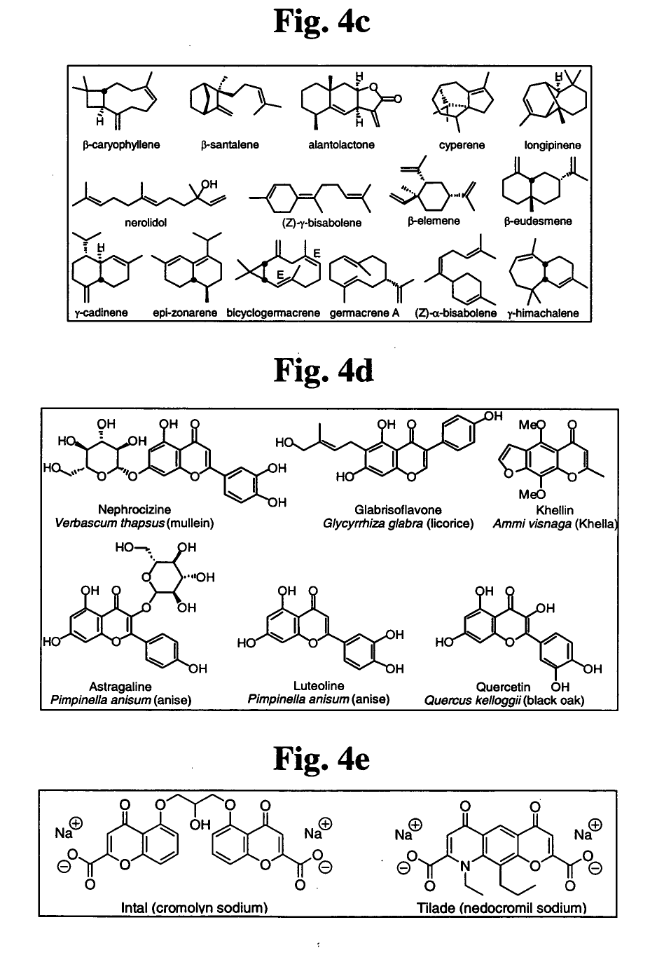

As is further described hereinabove, some of the presently known asthma medications include olefin compounds (see, for example, FIGS. 4d-f). Although these medications are not known to exert their therapeutic effect via inhibition of oxidant activity/formation, this and some other aspects of the present invention are not intended to encompass these compounds for the treatment of asthma.

Hence, in cases where the inflammation associated medical condition is asthma, the method according to this aspect of the present invention excludes any of theophylline, enprofylline, pentoxifylline, lisofylline, 3-isobutyl-1-methylxanthine (IBMX), caffeine, uric acid, khellin, cromolyn sodium, quercetin and/or nedocromil sodium.

As used herein, the term “alkyl” refers to a saturated aliphatic hydrocarbon including straight chain and branched chain groups. Preferably, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or unsubstituted. When substituted, the substituent group can be, for example, hydroxyalkyl, trihaloalkyl, cycloalkyl, alkenyl, alkynyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azo, sulfonyl, sulfinyl, sulfonamide, phosphonyl, phosphinyl, phosphonium, ketoester, carbonyl, thiocarbonyl, ester, ether, carboxy, thiocarboxy, thioether, thiocarbamate, urea, thiourea, O-carbamyl, N-carbamyl, O-thiocarbamyl, N-thiocarbamyl, C-amido, N-amido, C-carboxy, O-carboxy, sulfonamido, trihalomethanesulfonamido, guanyl, guanidino, carbohydrate, and amino, as these terms are defined herein.

A “cycloalkyl” group refers to an all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or unsubstituted. When substituted, the substituent group can be, for example, alkyl, hydroxyalkyl, trihaloalkyl, cycloalkyl, alkenyl, alkynyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azo, sulfonyl, sulfinyl, sulfonamide, phosphonyl, phosphinyl, phosphonium, ketoester, carbonyl, thiocarbonyl, ester, ether, carboxy, thiocarboxy, thioether, thiocarbamate, urea, thiourea, O-carbamyl, N-carbamyl, O-thiocarbamyl, N-thiocarbamyl, C-amido, N-amido, C-carboxy, O-carboxy, sulfonamido, trihalomethanesulfonamido, guanyl, guanidino, carbohydrate, and amino.

An “alkenyl” group refers to an alkyl group which consists of at least two carbon atoms and at least one carbon-carbon double bond.

An “aryl” group refers to an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl. The aryl group may be substituted or unsubstituted. When substituted, the substituent group can be, for example, alkyl, hydroxyalkyl, trihaloalkyl, cycloalkyl, alkenyl, alkynyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azo, sulfonyl, sulfinyl, sulfonamide, phosphonyl, phosphinyl, phosphonium, ketoester, carbonyl, thiocarbonyl, ester, ether, carboxy, thiocarboxy, thioether, thiocarbamate, urea, thiourea, O-carbamyl, N-carbamyl, O-thiocarbamyl, N-thiocarbamyl, C-amido, N-amido, C-carboxy, O-carboxy, sulfonamido, trihalomethanesulfonamido, guanyl, guanidino, carbohydrate, and amino.

A “heteroaryl” group refers to a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted. When substituted, the substituent group can be, for example, alkyl, hydroxyalkyl, trihaloalkyl, cycloalkyl, alkenyl, alkynyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azo, sulfonyl, sulfinyl, sulfonamide, phosphonyl, phosphinyl, phosphonium, ketoester, carbonyl, thiocarbonyl, ester, ether, carboxy, thiocarboxy, thioether, thiocarbamate, urea, thiourea, O-carbamyl, N-carbamyl, O-thiocarbamyl, N-thiocarbamyl, C-amido, N-amido, C-carboxy, O-carboxy, sulfonamido, trihalomethanesulfonamido, guanyl, guanidino, carbohydrate, and amino,

A “heteroalicyclic” group refers to a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. When substituted, the substituted group can be, for example, alkyl, hydroxyalkyl, trihaloalkyl, cycloalkyl, alkenyl, alkynyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azo, sulfonyl, sulfinyl, sulfonamide, phosphonyl, phosphinyl, phosphonium, ketoester, carbonyl, thiocarbonyl, ester, ether, carboxy, thiocarboxy, thioether, thiocarbamate, urea, thiourea, O-carbamyl, N-carbamyl, O-thiocarbamyl, N-thiocarbamyl, C-amido, N-amido, C-carboxy, O-carboxy, sulfonamido, trihalomethanesulfonamido, guanyl, guanidino, carbohydrate, and amino. Representative examples are piperidine, piperazine, tetrahydro furane, tetrahydropyrane, morpholino and the like.

A “hydroxy” group refers to an —OH group.

An “azo” group refers to a —N═N—R′ group, where R′ is hydrogen, alkyl, alkenyl, cycloalkyl, aryl, heteroaryl (bonded through a ring carbon) or heteroalicyclic (bonded through a ring carbon) as defined herein.

An “alkoxy” group refers to both an —O-alkyl and an —O-cycloalkyl group, as defined herein.

An “aryloxy” group refers to both an —O-aryl and an —O-heteroaryl group, as defined herein.

A “thiohydroxy” group refers to an —SH group.

A “thioalkoxy” group refers to both an —S-alkyl group, and an —S-cycloalkyl group, as defined herein.

An “thioaryloxy” group refers to both an —S-aryl and an —S-heteroaryl group, as defined herein.

A “carbonyl” group refers to a —C(═O)—R′ group, where R′ is hydrogen, alkyl, alkenyl, cycloalkyl, aryl, heteroaryl (bonded through a ring carbon) or heteroalicyclic (bonded through a ring carbon) as defined herein.

An “aldehyde” group refers to a carbonyl group, where R′ is hydrogen.

A “thiocarbonyl” group refers to a —C(═S)—R′ group, where R′ is as defined herein for R′.

A “C-carboxy” group refers to a —C(═O)—O—R′ groups, where R′ is as defined herein.

An “O-carboxy” group refers to an R″C(═O)—O— group, where R′ is as defined herein.

A “carboxy” group refers to a ═O group.

A “thiocarboxy” group referes to a ═S group.

A “carboxylic acid” group refers to a C-carboxyl group in which R′ is hydrogen.

An “ether” group refers to a —R′″—O—R′, were R′ is as defined hereinabove and R′″ is alkyl, alkenyl, cycloalkyl, aryl, heteroaryl (bonded through a ring carbon) or heteroalicyclic (bonded through a ring carbon) as defined herein.

A “thioether” group refers to a —R′″—S—R′, were R′ and R′″ are as defined herein.

A “halo” group refers to fluorine, chlorine, bromine or iodine.

A “trihaloalkyl” group refers to a —CX₃ group wherein X is a halo group as defined herein.

A “trihalomethanesulfonyl” group refers to an X₃CS(═O)₂— group wherein X is a halo group as defined herein.

A “sulfinyl” group refers to an —S(═O)—R′ group, where R′ is as defined herein.

A “sulfonyl” group refers to an —S(═O)₂—R′ group, where R′ is as defined herein.

An “S-sulfonamido” group refers to a —S(═O)₂—NR′R″ group, with R′ as defined hereinabove and R″ is as defined herein for R′.

An “N-sulfonamido” group refers to an R′S(═O)₂—NR″ group, where R′ and R″ are as defined herein.

A “trihalomethanesulfonamido” group refers to an X₃CS(═O)₂NR′— group, where R′ and X are as defined herein.

An “O-carbamyl” group refers to an —OC(═O)—NR′R″ group, where R′ and R″ are as defined herein.

An “N-carbamyl” group refers to an R″OC(═O)—NR′— group, where R′ and R″ are as defined herein.

An “O-thiocarbamyl” group refers to an —OC(═S)—NR′R″ group, where R′ and R″ are as defined herein.

An “N-thiocarbamyl” group refers to an R″OC(═S)NR′— group, where R′ and R″ are as defined herein.

An “Amino” group refers to an —NR′R″ group where R′ and R″ are as defined herein.

A “C-amido” group refers to a —C(═O)—NR′R″ group, where R′ and R″ are as defined herein.

An “N-amido” group refers to an R′C(═O)—NR″ group, where R′ and R″ are as defined herein.

An “urea” group refers to an —NR′C(═O)—NR″R′″ group, where R′ and R″ are as defined herein and R′″is defined as either R′ or R″.

A “guanidino” group refers to an —R′NC(═N)—NR″R′″ group, where R′, R″ and R′″ are as defined herein.

A “guanyl” group refers to an R′R″NC(═N)— group, where R′ and R″ are as defined herein.

A “nitro” group refers to an —NO₂ group.

A “cyano” group refers to a —C═N group.

The term “phosphonyl” describes a —O—P(═O)(OR′)— group, with R′ as defined hereinabove.

The term “phosphinyl” describes a —PR′— group, with R′ as defined hereinabove.

The term “phosphonium” is a —P+R′R″, where R′ and R″ are as defined hereinabove.

The term “ketoester” describes a —C(═O)—C(═O)—O— group.

The term “thiourea” describes a —NR′—C(═S)—NR′— group, with R′ and R″ as defined hereinabove.

Thus, each of X, Y, Z and W can be independently hydrogen or alkyl, such that the compound is an alkene, as defined hereinabove. Alternatively, two of X, Y, Z and W form a five- or six-membered alicyclic ring, such that the compound is a cycloalkene, namely, a cycloalkyl, as defined hereinabove, which has at least one carbon-carbon double bond within the ring, or a cycloalkane, as defined hereinabove, substituted by an alkene.

Preferred alkenes or cycloalkenes for use in the context of the present invention include those having between 2 and 15 carbon atoms, such as, but not limited to, ethylene, propylene, 1-butene, trans-2-butene, cis-2-butene, 2-methyl-2-butene, isoprene, butadiene, 2,3-dimethyl-2-butene, cyclohexene, cyclohexadiene and cyclopentene.

Such compounds are highly advantageous since they are non-toxic, hydrophobic and highly volatile (most of these compounds are gases at room temperature and at atmospheric pressure). The hydrophobicity of these compounds provides for enhanced membrane permeability thereof and thus facilitates their diffusion through membranes of the targeted tissue (e.g., bronchial and alveolar membranes). The volatility of these compounds enables their advantageous administration by inhalation, as is detailed hereinbelow.

As is well known in the art, the term “volatile” refers to a substance or a compound that has a relatively low boiling point, whereas a boiling point is defined as the temperature at which the vapor pressure of a substance is equal to the external pressure, e.g., an atmospheric pressure. The vapor pressure of a compound is inversely related to the energy that causes the molecules to attract one another, that is, if the intermolecular attractive force is week, little energy must be supplied in order to vaporization to occur and the compound has a high vapor pressure, a low boiling point and is therefore considered volatile.

Hence, the term “volatile” as used herein with respect to a compound refers to a compound that has a boiling point that ranges between about 30° C. and about 250° C., more preferably between about 30° C. and about 200° C., and more preferably between about 30° C. and about 150° C.

As used herein the term “about” refers to ±10%.

The volatile compounds described herein are further characterized by a relatively low molecular weight, which ranges between about 28 Da and about 1,000 Da, more preferably between about 28 Da and about 400 Da, more preferably between about 28 Da and about 300 Da, more preferably between about 28 Da and about 240 Da, more preferably between about 28 Da and about 180 Da. One of the presently preferred volatile compounds described herein has a molecular weight of about 136 Da.

In addition, some of these compounds, and particularly ethylene, are produced during the ripening process of various fruits (e.g., banana, citrus fruits and apples), and can therefore be administered via inhalation of such fruits, simply by placing ripening fruits next to asthma patients.

As is discussed hereinabove, and is well known in the art, electron rich olefins are more active oxidant scavengers than non-electron rich olefins. The electron enrichment is induced by the substituents on the carbon-carbon double bond (W, Y, Z and W in Formula VII hereinabove).

Hence, preferably, at least one of X, Y, Z and W is an electron-donating group.

Representative examples of electron-donating groups include, without limitation, alkyl, cycloalkyl, nitro, amino, hydroxy, alkoxy, aryloxy, thiohydroxy, thioalkoxy, as these terms are defined hereinabove, and aryl and heteroaryl, as defined hereinabove, substituted at the ortho and/or para positions by one or more of these electron donating group(s).

Compounds having a combination of electron-donating group(s) and electron-withdrawing group(s) are also preferred according to the present invention. Although less preferable, compounds having only electron withdrawing groups can also be included within the compounds of the present invention

Thus, further preferably, at least two of X, Y, Z and W are alkyl or cycloalkyl. Representative examples of such compounds are terpenes (e.g., monoterpenes and sesquiterpenes, as is shown in FIGS. 4b and 4c). Particularly preferred are monoterpenes, which are naturally occurring, non-toxic, hydrophobic and volatile compounds and therefore have the advantages described above for alkenes, with the addition of higher reactivity toward oxidation reactions. Monoterpenes can therefore also be administered by inhalation, which can be effected by placing ripening fruits next to a patient.

Representative examples of monoterpene compounds that are usable in the context of the present invention, include, without limitation, citronellol, geraniol, nerol, linalool, citral, carvone, pulegone, limonene, myrcene, α-terpinen, γ-terpinene, terpinolene, careen, terpinol, α-terpinol, α-thujene, lavandulol, α-pinene, β-pinene, myrtenol, camphene and rosoxide (see, FIG. 4b).

Although less volatile, sesquiterpene can also be utilized within embodiments of the present invention. Representative examples of such tsesquiterpenes include, without limitation, β-caryophyllene, β-santalene, alantolactone, cyperene, longipinene, nerolidol, (z)-γ-bisabolene, β-elemene, β-eudesmene, γ-cadinene, epi-zonarene, bicyclogemarcene, (z)-γ-bisabulene and γ-himachalene (see, FIG. 4c).

Further preferably, at least two of X, Y, Z and W are alkyl or alkenyl having at least 6 carbon atoms, whereas at least one of these alkyl or alkenyl terminates with a C(═O)-L group or a pharmaceutically acceptable salt thereof, and further wherein L is selected from the group consisting of hydrogen, alkyl, cycloalkyl, aryl, hydroxy, alkoxy, aryloxy and amino. Representative examples of such compounds are unsaturated fatty acids, e.g., linoleic acid, linolenic, acid, arachidonic acid, oleic acid and palmitoleic acid, and aldehydes, esters or amides derivatives thereof.

The use of this class of compounds is highly advantageous since fatty acids and derivatives thereof are non-toxic compounds and, furthermore, the products produced by their reaction with oxidants are known natural products.

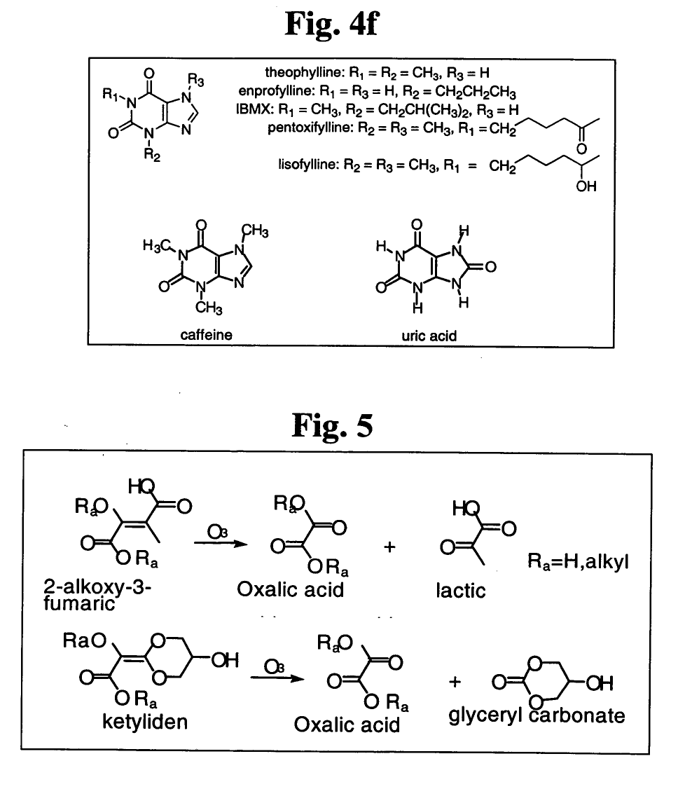

Further preferably, at least one of X, Y, Z and W is a C-carboxy group, as defined hereinabove, or a pharmaceutically acceptable salt thereof, such that the compound is an α,β-unsaturated carbonyl. As carboxylic groups are electron-withdrawing groups, preferred compounds in this class are those wherein at least one of X, Y, Z and W is an electron donating group, as described hereinabove. Preferred examples of such compounds include, for example, fumaric acid and ketylidene derivatives, which, as is shown in FIG. 5, decompose upon reacting with an oxidant into oxalic acid derivatives and lactic acid or glyceryl carbonate, respectively. These compounds therefore produce known blood components upon scavenging ozone, for example, and are thus highly advantageous.

Alternatively, the compounds are carbocyclic or heteroalicyclic unsaturated compounds, such that at least two of X, Y, Z and W form at least one five- or six-membered aromatic, heteroaromatic, alicyclic and/or heteroalicyclic ring, as these terms are defined hereinabove.

Representative examples of such compounds are those having the general Formulas I, II, III, V and VI below.

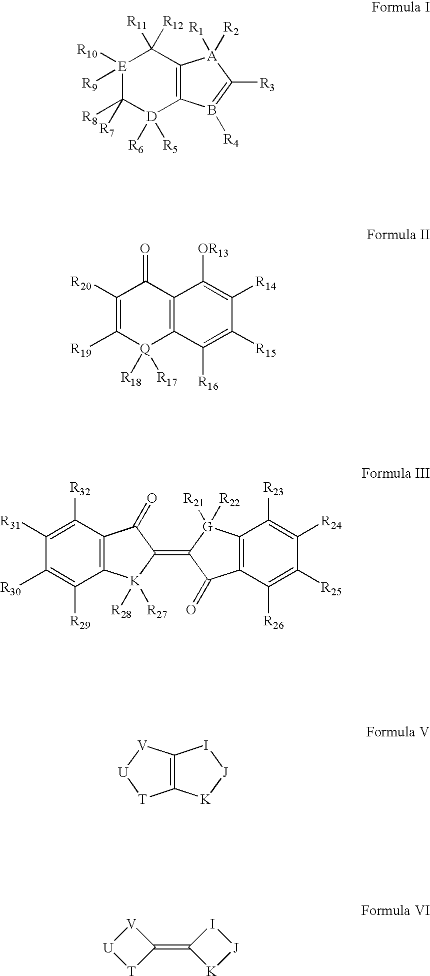

wherein: each of A, D, E, Q, G and K is independently selected from the group consisting of carbon, nitrogen, oxygen and sulfur; B is carbon or nitrogen; each of I, K, T and V is independently selected from the group consisting of CR₃₇R₃₈, NR₃₉, O and S; each of J and K is independnently selected from the group consisting of CR₄₀R₄₁, R₄₂R₄₃C—CR₄₄R₄₅, R₄₆R₄₇C—C(R₄₈R₄₉)—C(R₅₀R₅₁)—CR₅₂R₅₃ and R₅₄C═CR₅₅; and each of R₁-R₅₅ is independently selected from the group consisting of hydrogen, lone pair electrons, alkyl, hydroxyalkyl, trihaloalkyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azo, sulfonyl, sulfinyl, sulfonamide, phosphonyl, phosphinyl, phosphonium, ketone, ketoester, carbonyl, thiocarbonyl, ether, thiocarboxy, thioether, thiocarbamate, urea, thiourea, O-carbamyl, N-carbamyl, O-thiocarbamyl, N-thiocarbamyl, C-amido, N-amido, C-carboxy, O-carboxy, guanyl, guanidine and amino, or, alternatively, at least two of R₁-R₄, at least two of R₅-R₁₂, at least two of R₁₃-R₁₆, at least two of R₁₇-R₂₀, at least two of R₂₁-R₂₆, at least two of R₂₇-R₃₂ and/or at least two of R₃₇-R₅₅ form at least one five- or six-membered aromatic, heteroaromatic, alicyclic or heteroalicyclic ring, or a pharmaceutically acceptable salt thereof.

Compounds having the general Formula I above are xanthine derivatives or analogs. Thus, preferably, each of A, B, D and E is nitrogen and/or each of R₇ and R₁₁ is carbonyl and R₈ and R₁₂ are absent. Such compounds are highly reactive in oxidation reactions since the double bond is substituted by three electron-donating groups (—NH—).

Compounds having the general Formula II above are chromone derivatives or analogs. Preferably, Q is oxygen. The chromone derivatives according to the present invention can be bicyclic, tricyclic (where at least two of R₁₃-R₁₆ or at least two of R₁₇-R₂₀ form a ring), or tetracylic, where at least two of R₁₃-R₁₆ and at least two of R₁₇-R₂₀ form two rings, to thereby from four fuse rings, or, alternatively, where one of R₁₃-R₂₀ is a group substituted by another chromone derivative, to thereby form two bicyclic chromone derivatives connected one to the other via a bridging group.

Compounds having the general Formula II, wherein R₁₆ is hydroxyl, alkoxy, cycloalkoxy, aryloxy, thiohydroxy, thioalkoxy or thioaryloxy are highly preferable. Such compounds include a hydroquinone moiety, which, as is well known in the art, is a highly reactive oxidant scavenging group.

Compounds having the general Formula III above are indigoids. Preferably at least one of G and K is nitrogen or oxygen, such that the compounds have at least one electron donating group. Further preferably, at least one, and more preferably all, of R₂₃, R₂₆, R₂₉ and R₃₂ is hydroxyl, alkoxy, cycloalkoxy, aryloxy, thiohydroxy, thioalkoxy and/or thioaryloxy, such that the compound includes the reactive hydroquinone moiety described above.

Compounds having the general Formulas V and VI are known as organic conductors. These compounds typically exhibit electric conductivity in the solid state and are therefore also referred to as organic metals. These compounds typically include fused and non-fused heteroalicyclic or heteroaromatic bicyclic compounds (of five- or six-membered rings) or, alternatively, heteroalicyclic or heteroaromatic tetracyclic compounds of two fused bicyclic rings. These compounds are typically characterized by a carbon-carbon double bond substituted by four electron-donating groups and are therefore highly reactive oxidants scavengers.

Hence, preferred compounds that are capable of inhibiting the activity/formation of an oxidant by a stoichiometric scavenging of the oxidant, according to the present invention, include alkenes, terpenes, α,β-unsaturated carbonyls, xanthines, chromones, unsaturated fatty acids, indigoids, organic conductors and any derivatives or analogs thereof, as is detailed hereinabove.

Most of these compounds are hydrophobic and/or lipophilic and therefore can easily penetrate the membranes of the targeted tissues, where inflammation occurs (e.g., the bronchial and alveolar membranes).

As is described hereinabove, scavenging the oxidant can alternatively be performed by a catalytic reaction of the compound with the oxidant. Representative examples of compounds that can catalytically react with oxidants such as ozone include organometallic compounds that are susceptible to oxidation reactions, such as, but not limited to metalloporphyrins, derivatives, analogs and pharmaceutically acceptable salt thereof.

Additional compounds in this class include, for example, organometallic complexes of metallic elements such as, for example, Zn (II), Cu (II), Fe(II), La (III), Lu (III), Y (III), In (III) Cd (II), Mg (II), Al(III) and Ru, and organic chelators such as porphyrins and polyamines (e.g., ethylene diamine and cyclam). As is further described hereinabove, the compounds according to the present invention may be selected capable of inhibiting an oxidant-producing biochemical pathway of the present invention. The compounds may be selected capable of inhibiting, via any of various mechanisms, any of various oxidant-producing biochemical pathways of the present invention, depending on the application and purpose.

Preferably, the compounds are selected capable of inhibiting an enzymatic activity and/or formation of a protein which mediates the biochemical pathway.

Examples of proteins which mediate biochemical pathways that produce an oxidant of the present invention, such as ozone, particularly include antibodies. It has been shown that antibodies, essentially regardless of their antigenic specificities, mediate ozone production via a water oxidation pathway (Wentworth, Jr. et al., 2002. Science 298:2195).

The compounds described herein may advantageously include any of various types of molecules routinely employed by the ordinarily versed artisan for inhibiting expression of a protein, such as an antibody, so as to thereby inhibit formation thereof, and hence to inhibit an activity thereof, such as mediation of a biochemical pathway producing an oxidant of the present invention such as ozone. Particularly effective instances of such compounds include those which are capable of preventing expression of the protein from a nucleic acid sequence encoding the protein. Such compounds include, but are not limited to, “small interfering RNAs” (siRNAs), antisense polynucleotides, DNAzymes, ribozymes and triplex-forming oligonucleotides (TFOs).

Small interfering RNAs can be used to prevent translation of essentially any selected target protein from an RNA molecule encoding the protein, via the two-step process of RNA interference (RNAi). In the first step, termed the initiation step, input dsRNA is digested into 21-23 nucleotide (nt) small interfering RNAs (siRNA), by the action of Dicer, a member of the RNase III family of dsRNA-specific ribonucleases, which processes (cleaves) dsRNA (introduced directly or via a transgene or a virus) in an ATP-dependent manner. Successive cleavage events degrade the RNA to 19-21 bp duplexes (siRNA), each with 2-nucleotide 3′ overhangs [Hutvagner and Zamore Curr Opin Genet Dev. 12:225-232 (2002); and Bernstein, Nature 409:363-366 (2001)].

In the effector step, the siRNA duplexes bind to a nuclease complex to from the RNA-induced silencing complex (RISC). An ATP-dependent unwinding of the siRNA duplex is required for activation of the RISC. The active RISC then targets the homologous transcript by base pairing interactions and cleaves the mRNA into 12 nucleotide fragments from the 3′ terminus of the siRNA [Hutvagner and Zamore, Curr Opin Genet Dev. 12:225-232 (2002); Hammond et al., 2001. Nat Rev Gen. 2:110-119 (2001); and Sharp Genes Dev. 15:485-90 (2001)]. Although the mechanism of cleavage is still to be elucidated, research indicates that each RISC contains a single siRNA and an RNase [Hutvagner and Zamore, Curr Opin Genet Dev. 12:225-232 (2002)].

Because of the remarkable potency of RNAi, an amplification step within the RNAi pathway has been suggested. Amplification could occur by copying of the input dsRNAs which would generate more siRNAs, or by replication of the siRNAs formed. Alternatively or additionally, amplification could be effected by multiple turnover events of the RISC [Hammond et al., Nat Rev Gen. 2:110-119 (2001), Sharp Genes Dev. 15:485-90 (2001); Hutvagner and Zamore, Curr Opin Genet Dev. 12:225-232 (2002)]. Ample guidance for using RNAi to practice the present invention is provided in the literature of the art [refer, for example, to: Tuschl, ChemBiochem. 2:239-245 (2001); Cullen, Nat Immunol. 3:597-599 (2002); and Brantl, Biochem Biophys Acta 1575:15-25 (2002)].

Synthesis of RNAi molecules suitable for use with the present invention can be effected as follows. First, the mRNA sequence encoding the protein involved in the oxidant-producing biochemical pathway is scanned downstream of the AUG start codon for AA dinucleotide sequences. Occurrence of each amino acid residue and the 3′ adjacent 19 nucleotides is recorded as potential siRNA target sites. Preferably, siRNA target sites are selected from the open reading frame, as untranslated regions (UTRs), being enriched in regulatory protein binding sites. UTR-binding proteins and/or translation initiation complexes may interfere with binding of the siRNA endonuclease complex [Tuschl, Chem Biochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www.ambion.com/techlib/tn/91/912.html).

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

As described hereinabove, the compounds described herein can include an antisense polynucleotide, in particular an antisense polynucleotide which is capable of specifically hybridizing with an mRNA transcript encoding the protein so as to inhibit expression of essentially any selected target protein from an RNA molecule. Such an antisense polynucleotide can be designed so as to be capable of specifically inhibiting expression of essentially any selected target protein.

Design of antisense polynucleotides which can be used to efficiently inhibit expression of a target protein must be effected while considering two aspects important to the antisense polynucleotide approach. The first aspect is delivery of the antisense polynucleotide into the cytoplasm of the appropriate cells, while the second aspect is design of an antisense polynucleotide which specifically binds the designated mRNA within cells in a way which inhibits translation thereof.

The literature of the art teaches of a number of suitable delivery strategies which can be used to efficiently deliver polynucleotides, such as antisense polynucleotides, into a wide variety of cell types [see, for example: Luft, J Mol Med. 76:75-6 (1998); Kronenwett et al., Blood 91:852-62 (1998); Rajur et al., Bioconjug Chem. 8:935-40 (1997); Lavigne et al., Biochem Biophys Res Commun. 237:566-71 (1997) and Aoki et al., (1997) Biochem Biophys Res Commun. 231:540-5 (1997)].

In addition, algorithms for identifying antisense polynucleotide sequences with the highest predicted binding affinity for their target mRNA based on a thermodynamic cycle that accounts for the energetics of structural alterations in both the target mRNA and the antisense polynucleotide are also available [see, for example, Walton et al., Biotechnol Bioeng 65:1-9 (1999)]. Such algorithms have been successfully used to implement an antisense polynucleotide-based protein translation approach in cells. For example, the algorithm developed by Walton et al., enabled scientists to successfully design antisense polynucleotides for rabbit beta-globin and mouse tumor necrosis factor-alpha transcripts. The same research group has also reported that the antisense activity of rationally selected antisense polynucleotides against various model target mRNAs in cell culture proved effective, including tests against three different targets in two cell types with antisense polynucleotides having phosphodiester and phosphorothioate chemistries.

In addition, several approaches for designing and predicting efficiency of specific antisense polynucleotides using an in vitro system were also published [Matveeva et al., Nature Biotechnology 16:1374-1375 (1998)].

Several clinical trials have demonstrated the safety, feasibility and effective of using antisense polynucleotides for inhibiting expression of specific target proteins. For example, antisense polynucleotides suitable for the treatment of cancer have been successfully used [Holmund et al., Curr Opin Mol Ther. 1:372-85 (1999)], while treatment of hematological malignancies via antisense polynucleotides targeting c-myb gene, p53 and Bcl-2 had entered clinical trials and had been shown to be tolerated by patients [Gerwitz, Curr Opin Mol Ther. 1:297-306 (1999)]. Furthermore, antisense polynucleotide-mediated suppression of human heparanase gene expression has been used to inhibit pleural dissemination of human cancer cells in a mouse model [Uno et al., Cancer Res 61:7855-60 (2001)].

Thus, the current consensus is that recent developments in the field of antisense polynucleotide technology which, as described above, have led to the generation of highly accurate antisense design algorithms and a wide variety of antisense polynucleotide delivery systems, enable an ordinarily skilled artisan to design antisense polynucleotides and implement delivery approaches thereof suitable for inhibiting protein expression from known mRNA sequences without having to resort to undue trial and error experimentation.

As described hereinabove, the compounds may advantageously include a DNAzyme. A DNAzyme is a molecule which is capable of specifically cleaving an mRNA transcript or DNA sequence of essentially any selected target protein, such as a protein mediating a biochemical pathway producing an oxidant of the present invention. DNAzymes are single-stranded polynucleotides which are capable of cleaving both single and double stranded target sequences (Breaker, R. R. and Joyce, G. Chemistry and Biology, 1995, 2:655; Santoro, S. W. and Joyce, G. F. Proc. Natl. Acad. Sci. U.S.A., 1997, 943:4262). A general model (the “10-23” model) for the DNAzyme has been proposed. Namely, “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions [Santoro, S. W. and Joyce, G. F. Proc. Natl. Acad. Sci. U.S.A., 1997, 943:4262; for review of DNAzymes, refer, for example, to Khachigian, LM., Curr Opin Mol Ther. 4:119-21 (2002)].

Examples of construction and amplification of synthetic, engineered DNAzymes recognizing single and double-stranded target cleavage sites have been disclosed in U.S. Pat. No. 6,326,174 to Joyce et al., in which DNAzymes of similar design directed against the human urokinase receptor were recently observed to inhibit urokinase receptor expression, and successfully inhibit colon cancer cell metastasis in vivo (Itoh et al., 20002, Abstract 409, Ann Meeting Am Soc Gen Ther., http://www.asgt.org). In another application, DNAzymes complementary to bcr-abl oncogenes were successful in inhibiting the oncogenes expression in leukemia cells, and lessening relapse rates in autologous bone marrow transplant in cases of CML and ALL.

As described hereinabove, the compounds described herein may advantageously include a ribozyme. A ribozyme is a molecule which is capable of specifically cleaving an mRNA transcript encoding a selected target protein, such as a protein mediating a biochemical pathway producing an oxidant of the present invention [Welch et al., Curr Opin Biotechnol. 9:486-96 (1998)]. In the therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)]. The effectiveness of ribozymes has also been demonstrated in studies involving transgenic animals, gene target validation or biochemical pathway elucidation. For example, the ribozyme HEPTAZYME (Ribozyme Pharmaceuticals, Inc.), which was designed for destroying hepatitis C virus RNA, has been shown to be effective in decreasing hepatitis C virus RNA in cell culture assays. Importantly, ribozymes have been employed in human clinical trials. For example, the ribozyme ANGIOZYME, which specifically inhibits formation of vascular endothelial growth factor receptor, a key component of the angiogenesis pathway, has been employed in human clinical trials. Ribozymes have also been approved for use in several clinical trials involving ribozyme gene therapy for HIV/AIDS patients.

As described hereinabove, the compounds described herein may advantageously include a triplex-forming oligonucleotide (TFO). A TFO is an oligonucleotide designed to recognize and bind to specific polypurine/polypyrimidine regions, which are generally present in promoter sequences in double-stranded DNA, so as to interfere with transcription of coding sequences under the regulatory control of the targeted promoter. As such TFOs can be used for inhibiting expression of promoter-regulated gene products.

In general, the triplex-forming oligonucleotide has the sequence correspondence: [oligo, 3′-A G G T]::[duplex, 5′-A G C T]::[duplex, 3′-T C G A], however, it has been shown that the A-AT and G-GC triplets have the greatest triple helical stability (Reither and Jeltsch, BMC Biochem, 2002, Sept. 12, Epub). The same authors have demonstrated that TFOs designed according to the A-AT and G-GC rule do not form non-specific triplexes, indicating that the triplex formation is indeed sequence specific. Modification of oligonucleotides, such as the introduction of intercalators and backbone substitutions, and optimization of binding conditions (pH and cation concentration) can be used for overcoming charge repulsion and instability obstacles to TFO activity, and synthetic oligonucleotides can be targeted to specific sequences (refer, for example, to Seidman and Glazer, J Clin Invest. 2003, 112:487-94).

Thus for any given sequence in the promoter of a gene encoding a selected target gene product, a triplex forming sequence may be devised. Triplex-forming oligonucleotides preferably are at least 15, more preferably 25, still more preferably 30 or more nucleotides in length, up to 50 or 100 bp.

Transfection of cells (for example, via cationic liposomes) with TFOs, and formation of the triple helical structure with the target DNA induces steric and functional changes, blocking transcription initiation and elongation, allowing the introduction of desired sequence changes in the endogenous DNA and resulting in the specific downregulation of gene expression. Examples of such suppression of gene expression in cells treated with TFOs include knockout of episomal supFGI and endogenous HPRT genes in mammalian cells (Vasquez et al., Nucl Acids Res., 1999, 27:1176-81; and Puri et al, J Biol Chem., 2001, 276:28991-98), and the sequence- and target-specific downregulation of expression of the Ets2 transcription factor, important in prostate cancer etiology (Carbone et al., Nucl Acid Res., 2003, 31:833-43), and the proinflammatory ICAM-1 gene (Besch et al., J Biol Chem., 2002, 277:32473-79).

Ample guidance for designing, obtaining and using TFOs for effectively practicing the treatment method of the present invention is provided in the literature of the art (refer, for example, to: Maher III, L. J. et al., Science, 1989, 245:725-730; Moser, HE. et al., Science, 1987, 238:645-630; Beal, P A., et al, Science, 1992, 251:1360-1363; Cooney, M. et al., Science, 1988, 241:456-459; and Hogan, M E. et al., EP Publication No. 375408; U.S. Patent Application Nos. 2003 017068 and 2003 0096980 to Froehler et al., U.S. Patent Application Nos. 2002 0128218 and 2002 0123476 to Emanuele et al., and U.S. Pat. No. 5,721,138 to Lawn).

It will be appreciated that that for the treatment of an inflammatory pulmonary disease, such as asthma or chronic obstructive pulmonary disease, the compound(s) can be administered, according to need, in combination with a muscarinic receptor antagonist (e.g. ipatropium bromide or tiotropium) or a steroidal anti-inflammatory agent (e.g. fluticasone propionate, beclomethasone, budesonide, mometasone, ciclesonide, or triamcinolone). In addition, the compound(s) can be co-administered with an agent having anti-inflammatory and/or bronchodilating or other beneficial activity, including but not limited to, a phosphodiesterase (PDE) inhibitor (e.g. theophylline); a PDE4 inhibitor (e.g. cilomilast or roflumilast); an immunoglobulin antibody (anti-IgE antibody); a leukotriene antagonist (e.g. monteleukast); a cytokine antagonist therapy, such as, an anti-interleukin (IL) antibody, specifically, an anti-IL-4 antibody, an anti-IL-13 antibody, or a combination thereof; a protease inhibitor, such as an elastase or tryptase inhibitor; cromolyn sodium; nedocromil sodium; and sodium cromoglycate. It will be further appreciated that, the compound(s) can be co-administered with an antiinfective agent or an antihistamine, where appropriate.

The compounds described herein may be administered to the subject in any of various ways, depending on the application and purpose. In particular, according to the present invention, the compound(s) may be administered via any of various routes, according to any of various timing regimens, for a duration of any of various time periods, and according to any of various dosing regimens so as to achieve optimal treatment of the medical condition in the subject. One of ordinary skill in the art, preferably a physician, more preferably a physician specialized in the medical condition, will possess the necessary skill for administering the compound(s) according to the teachings of the present invention so as to achieve optimal treatment of the medical condition in the subject. Guidance for administering the compound(s) in such a way as to achieve optimal treatment of the medical condition in the subject is provided hereinbelow.

Since, as described hereinabove, the compounds described herein are preferably used to treat an inflammatory pulmonary disease such as asthma, the compound is preferably administered via inhalation. It will be appreciated that administration via inhalation will be optimal for achieving delivery of the compound to airway membranes, and hence for treating a disease such as asthma which is associated with airway membrane inflammation. Various medical conditions which are associated with inflammation in airway membranes which are effectively treatable according to this aspect of the present invention are described hereinbelow.

For treating inflammation connected with arthritis, a preferred mode of administration is topical application and/or intradermal injection.

As described hereinabove, the compound(s) described herein may be administered for a duration of any of various time periods; and may be advantageously administered during the time period according to a substantially continuous, discontinuous and/or mixed substantially continuous/discontinuous administration regimen.

Administration of compounds described herein via a mixed substantially continuous/discontinuous administration regimen is preferably effected via substantially continuous administration of the compound(s) for a duration of 6-12 hours daily, more preferably for a duration of about 8 hours daily. Preferably, such duration corresponds to the sleeping hours of the subject being treated.

Administering to the subject the compounds described herein may be effected at least once daily during any of various time periods, depending on the application and purpose. The duration of such a time period may be selected from any of the following: about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, about 31 days, about 32 days, about 33 days, about 34 days, about 35 days, about 36 days, about 37 days, about 38 days, about 39 days, about 40 days, about 41 days, about 42 days, about 43 days, about 44 days, about 45 days, about 46 days, about 47 days, about 48 days, about 49 days, about 50 days, about 51 days, about 52 days, about 53 days, about 54 days, about 55 days, about 56 days, about 57 days, about 58 days, about 59 days, about 60 days, about 61 days, about 62 days, about 63 days, about 64 days, about 65 days, about 66 days, about 67 days, about 68 days, about 69 days, about 70 days, about 71 days, about 72 days, about 73 days, about 74 days, about 75 days, about 76 days, about 77 days, about 78 days, about 79 days, about 80 days, about 81 days, about 82 days, about 83 days, about 84 days, about 85 days, about 86 days, about 87 days, about 88 days, about 89 days, about 90 days, about 91 days, about 92 days, about 93 days, about 94 days, about 95 days, about 96 days, about 97 days, about 98 days, about 99 days, about 100 days, about 101 days, about 102 days, about 103 days, about 104 days, about 105 days, about 106 days, about 107 days, about 108 days, about 109 days, about 110 days, about 111 days, about 112 days, about 113 days, about 114 days, about 115 days, about 116 days, about 117 days, about 118 days, about 119 days, about 120 days, about 121 days, about 122 days, about 123 days, about 124 days, about 125 days, about 126 days, about 127 days, about 128 days, about 129 days, about 130 days, about 131 days, about 132 days, about 133 days, about 134 days, about 135 days, about 136 days, about 137 days, about 138 days, about 139 days, or about 140 days.

The phrase “at least once daily” when relating to administration of compounds described herein for a time period of less than one day refers to a single administration of such compounds during the time period.

According to preferred embodiments, depending on the application and purpose, the time period is about 20 hours, about 7 days or about 14 days.

It will be appreciated that the compounds described herein may be administered for an indefinite time-period, if necessary, for example for treating a medical condition which is chronic.

Substantially continuous administration of the compounds described herein may be achieved using any of various commonly employed techniques, including via slow-release implant, slow-release capsules, intravenous drip infusion, dermal-patch, provision of an ambient breathing atmosphere containing the compound(s), etc.

As described hereinabove, the compounds described herein may be administered to the subject according to any of various dosing regimens.

The compound(s) may be administered to the subject in any of various therapeutically effective amounts, depending on the application and purpose. Guidance regarding determination of a suitable therapeutically effective amount of the compound(s) for treating a medical condition in a subject according to the teachings of the present invention is provided hereinbelow.

According to the teachings of the present invention, the therapeutically effective amount of a compound described herein may be selected so as to achieve any of various concentrations thereof at a site of the inflammation following administration thereof. Such a concentration may be selected from any of the following ranges: about 10 ppb to about 1,250 ppm, about 10 ppm to about 100 ppm, about 100 ppm to about 200 ppm, about 200 ppm to about 300 ppm, about 300 ppm to about 400 ppm, about 400 ppm to about 300 ppm, about 400 ppm to about 500 ppm, about 600 ppm to about 700 ppm, about 700 ppm to about 800 ppm, about 900 ppm to about 1,000 ppm, about 1,000 ppm to about 1,100 ppm, about 1,100 ppm to about 1,200 ppm, about 8 ppm to about 1,250 ppm, about 8 ppm to about 800 ppm, about 20 ppm to about 400 ppm, about 40 ppm to about 200 ppm, about 60 ppm to about 100 ppm, about 12 ppm to about 1,250 ppm, about 25 ppm to about 1,000 ppm, about 50 ppm to about 750 ppm, about 75 ppm to about 500 ppm, about 100 ppm to about 250 ppm, about 100 ppm to about 150 ppm, or about 80 ppm to about 125 ppm.

According to one preferred embodiment, the therapeutically effective amount of a compound described herein may be selected so as to achieve a concentration thereof of about 80 ppm at a site of the inflammation following administration thereof.

According to another preferred embodiment, the therapeutically effective amount of a compound described herein may be selected so as to achieve a concentration thereof of about 125 ppm at a site of the inflammation following administration thereof.

As is described in Example 1 of the Examples section below, substantially continuous exposure of airway surfaces of a treated mammalian subject to a compound of the present invention for a time period of 20 hours can be used to effectively treat an inflammatory pulmonary disease such as asthma in the subject.

As is described in Example 1 of the Examples section below, substantially continuous exposure of airway surfaces of a treated mammalian subject to a compound of the present invention, such as d-limonene, at a concentration of about 125 ppm for a time period of 7 days can be used to effectively treat an inflammatory pulmonary disease such as asthma in the subject.

As is described in Example 2 of the Examples section below, nightly exposures of airway surfaces of a treated human subject to a compound of the present invention, such as d-limonene, at a concentration of about 80 ppm for a time period of 14 days can be used to effectively treat an inflammatory pulmonary disease such as asthma in the subject.

The compounds described herein can be administered per se or comprised as a active ingredients in a pharmaceutical composition which further comprises a pharmaceutically acceptable carrier.

Thus, according to the present invention there is provided a pharmaceutical composition identified for use in the treatment of the medical condition, comprising, as an active ingredient, the compounds described herein, and a pharmaceutically acceptable carrier.

As used herein the phrase “pharmaceutical composition” refers to a preparation of one or more of the active ingredients described herein with other chemical components, such as physiologically suitable carriers and excipients. The purpose of a pharmaceutical composition is to facilitate administration of active ingredients to an organism, and/or to achieve a desired pharmacological effect of the active ingredients, preferably both.

As used herein the phrase “active ingredient” refers to the compounds described herein accountable for the biological/therapeutic effect.

Hereinafter, the phrases “physiologically acceptable carrier” and “pharmaceutically acceptable carrier,” which may be interchangeably used, refer to a carrier or a diluent that does not cause significant irritation to an organism, and does not abrogate the biological activity and properties of the administered active ingredients. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols. The pharmaceutical composition may advantageously take the form of a foam or a gel.

Techniques for formulation and administration of drugs (i.e. the at least one compound according to this aspect of the present invention) may be found in “Remington's Pharmaceutical Sciences,” Mack Publishing Co., Easton, Pa., latest edition, which is incorporated herein by reference.

Suitable routes of administration include any of various systemic and/or local routes.

Suitable routes of administration may, for example, include the inhalation, oral, buccal, rectal, transmucosal, topical, transdermal, intradermal, transnasal, intestinal and/or parenteral routes; the intramuscular, subcutaneous and/or intramedullary injection routes; the intrathecal, direct intraventricular, intravenous, intraperitoneal, intranasal, and/or intraocular injection routes; and/or the route of direct injection into a tissue region of a subject of the present invention.

Administration of the at least one compound via a suitable route, for example, via the inhalation route where the medical condition is an inflammatory pulmonary disease, enables administration specifically to the cells/tissues which are most affected by the disease. Hence, such a properly selected administration route enables administration of minimal doses of the at least one compound, thereby minimizing the risk of potential harmful side-effects.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

Depending on the application and purpose, the pharmaceutical composition may advantageously include as a formulating agent a suspending agent, a stabilizing agent, a dispersing agent, a foaming agent and a gelling agent.

For administration via the inhalation route, the active ingredients for use according to the present invention are preferably delivered using an inhalation device, as described hereinbelow.

For administration via injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active ingredients with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active ingredient doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredients may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

For administration via the topical route, the pharmaceutical composition be advantageously comprised in a cream or in a skin pad.

Pharmaceutical compositions suitable for use in context of the present invention include compositions wherein the active ingredients are contained in an amount effective to achieve the intended purpose. More specifically, a therapeutically effective amount means an amount of the at least one compound effective for treating the medical condition in the subject.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition (see e.g., Fingl, et al., 1975, in “The Pharmacological Basis of Therapeutics,” Ch. 1, p. 1).

Dosage amount and interval may be adjusted individually to provide plasma or brain levels of the active ingredients which are sufficient to achieve therapeutic effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or of a plurality of administrations, with course of treatment being of any of various suitable durations, as described above.

The amount of the composition to be administered will be dependent on parameters specific to the subject being treated, the severity of the medical condition, the manner of administration, the judgment of the prescribing physician, etc.

Preferably, a dose-unit of the pharmaceutical composition is selected so as to achieve at a site of the inflammation associated with the medical condition, a concentration of the at least one compound suitable for treatment of the medical condition, as described hereinabove.

The pharmaceutical composition may be included in a pack or dispenser device, such as an FDA approved kit, which may contain one or more unit dosage forms containing the active ingredients. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as described above.

Preferably, the pharmaceutical composition is packaged in a packaging material and identified in print, in or on the packaging material, for use in the treatment of the medical condition.

As described hereinabove, the at least one compound may be comprised in an inhalation device for administration to the respiratory tract of the subject.

Thus, according to the present invention there is provided an inhalation device for use in the treatment of the medical condition, comprising at least one of the compounds described hereinabove and a respiratory delivery system.

The inhalation device may be based on any of various suitable types of respiratory delivery systems which are suitable for administering a therapeutically effective dose of the at least one compound to the respiratory tract of a subject of the present invention. The inhalation device may be configured to deliver to the respiratory tract of the subject, preferably via the oral and/or nasal route, the at least one compound in the form of an aerosol/spray, a vapor and/or a dry powder mist. Numerous respiratory systems and methods of incorporating therapeutic agents therein, such as the at least one compound of the present invention, suitable for assembly of a suitable inhalation device according to the present invention are widely employed by the ordinarily skilled artisan and are extensively described in the literature of the art (refer, for example to U.S. Pat. Nos. 6,566,324, 6,571,790, 6,637,430, and 6,652,323; U.S. Food & Drug Administration (USFDA) Center For Drug Evaluation and Research (CDER); http://www.mece.ualberta.ca/arla/tutorial.htm).

Depending on the application and purpose, the respiratory delivery system may thus be, for example, a nebulizer inhaler, a dry powder inhaler (DPI), a metered dose inhaler (MDI), an oil warmer, a vaporizer and/or an atomizer.

Metered-dose inhalers typically discharge a measured amount of a therapeutic agent from a pressurized cannister (for example, Serevent® Inhalational Aerosol) using a compressed propellant gas. Typically, a human individual self-administers a therapeutic agent via an MDI by applying pressure to a trigger on the MDI so as to deliver a “burst” of a mixture of propellant and medicament into the mouth during an inhalation, the propelling “burst” being provided by the pressure within the cannister. A fixed number of doses are available in a given MDI. When all of the medicament has been dispensed from the cannister, typically the MDI or at least the cannister of medicament/propellant is discarded. Suitable formulations for MDI administration of a therapeutic agent include a solution or suspension of the therapeutic agent in a liquefied propellant. Suitable liquefied propellants include chlorofluorocarbons (CFCs), such as trichlorofluoromethane dichlorodifluoromethane, and dichloro-tetrafluoroethane however formulations using hydrofluoroalkanes (HFA), such as 1,1,1,2-tetrafluoroethane (HFA 134a) and 1,1,1,2,3,3,3,-heptafluoro-n-propane, (HFA 227) may be preferred due their avoidance of atmospheric ozone-depletion, unlike chlorofluorocarbons. Additional components of HFA formulations for MDI administration may advantageously include co-solvents, such as ethanol or pentane, and surfactants, such as sorbitan trioleate, oleic acid, lecithin, or glycerin (refer, for example, to U.S. Pat. No. 5,225,183, EP 0717987 A2, and PCT Publication No. WO 92/22286). Thus, a suitable formulation for MDI administration can include from about 0.01% to about 5% by weight of a pharmaceutical salt of a therapeutic agent, from about 0% to about 20% by weight ethanol, and from about 0% to about 5% by weight surfactant, with the remainder being the HFA propellant. In one approach, to prepare the formulation, chilled or pressurized hydrofluoroalkane is added to a vial containing a pharmaceutical salt of therapeutic agent, ethanol (if present) and the surfactant (if present). To prepare a suspension, the pharmaceutical salt may be provided as micronized particles. The formulation may be loaded into an aerosol canister, which forms a component of an MDI. Examples of MDIs developed specifically for use with HFA propellants are described in U.S. Pat. Nos. 6,006,745 and 6,143,277. In an alternative preparation, a suspension formulation is prepared by spray-drying a coating of surfactant on micronized particles of a pharmaceutical salt of a therapeutic agent (see, for example; PCT Publication Nos. WO 99/53901 and WO 00/61108). For additional examples of processes of preparing respirable particles, and formulations and devices suitable for inhalation-dosing see, for example, U.S. Pat. Nos. 6,268,533, 5,983,956, 5,874,063, and 6,221,398; and PCT Publication Nos. WO 99/55319 and WO 00/30614. MDIs are advantageous in cases where an easily portable hand-held device is desired. Conventional MDIs can be modified so as to increase the ability to obtain repeatable dosing by utilizing technology which measures the inspiratory volume and flow rate of a subject (refer, for example, to U.S. Pat. Nos. 5,404,871 and 5,542,410).

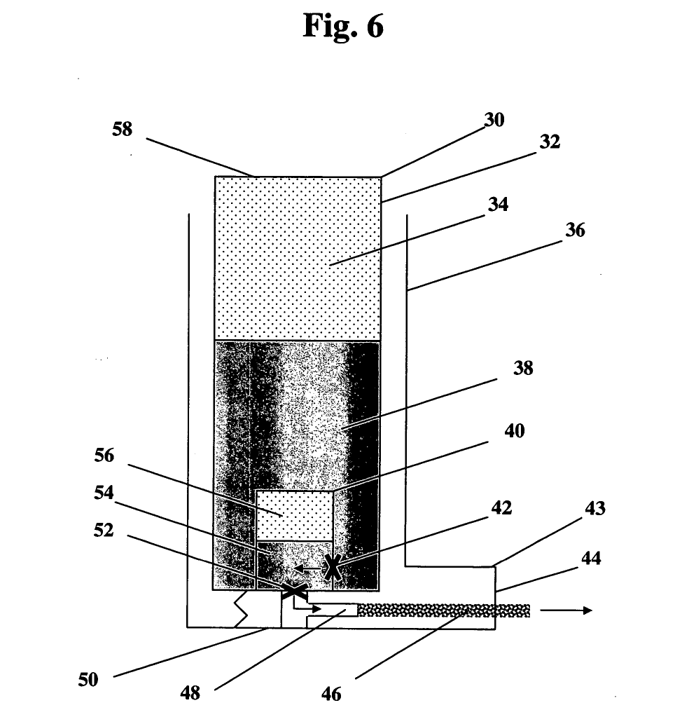

An example of a hand-held MDI according is shown in FIG. 6. Metered-dose inhaler 30 is composed of pressurized cartridge 32 connected to spray-nozzle 48 via housing 36. Pressurized cartridge 32 contains liquid 38, which includes a mixture of liquid-phase propellant and therapeutic agent; pressurized propellant vapor 34, and dose-chamber 40. Dose-chamber 40 contains: liquid 54, which includes a mixture of liquid-phase propellant and therapeutic agent; pressurized propellant vapor 56; outlet valve 52. Valve 52 is configured so as to open upon manual squeezing of cartridge surface 58 and housing surface 50 toward each other, and to close the absence of the squeezing. Inversely, valve 42 is configured to as to close during the squeezing and to open in the absence of the squeezing. Liquid 38 contains the therapeutic agent at a concentration such that the total volume of liquid 54, essentially representing an aliquot of liquid 38, contains a metered dose of therapeutic agent. For administration of the metered dose, the mouth of the subject is sealed around mouthpiece 43, and surfaces 58 and 50 are squeezed together manually by the subject concomitantly with inhalation by the subject. The squeezing opens valve 52, allowing pressurized vapor 56 to drive liquid 54 through spray-nozzle 48, thereby forming aerosol 46. The metered dose, contained in aerosol 46, is delivered via the inhaled air-stream to the respiratory tract of the subject via mouthpiece 43. Termination of the squeezing allows outlet valve 52 to close, and intake valve 42 to open so as to replenish dose-chamber 40 with liquid 54 and vapor 56 in preparation of administration of a subsequent metered dose of the therapeutic agent.

Nebulizer inhalers produce a stream of high velocity gas, typically from a pressurized external source, that causes a therapeutic agent to spray as a mist which is carried into the respiratory tract of a subject. The therapeutic agent is formulated in a liquid form, such as a solution or a suspension of micronized particles of respirable size, where a suspension of micronized particles is defined as being composed of at least 90% of particles with a diameter of about 10 microns or less. Nebulizer inhalers are most adapted for use in a clinic or hospital setting. At the end of the treatment or upon the consumption of all of the therapeutic agent solution in the nebulizer inhaler, and typically following a sterilization procedure, the nebulizer inhaler is replenished with therapeutic agent solution for use in further treatment. A typical formulation of a therapeutic agent for use in a nebulizer inhaler is an isotonic aqueous solution of a pharmaceutical salt of the therapeutic agent at a concentration between about 0.05 micrograms/ml and about 10 mg/ml. Types of nebulizer inhalers well-known and widely employed in the art include venturi type nebulizer inhalers or ultrasonic nebulizer inhalers Nebulizer inhalers are advantageous for achieving substantially continuous administration of therapeutic agent on a time-scale of minutes, or longer, and may be preferred for subjects who are unable to coordinate inhalational effort, and/or who are unable to master the technique of using hand-held MDIs, such as for example, infants, young children, debilitated individuals, and animals. For self-administering a preset continuous dose of a therapeutic agent via a nebulizer inhaler, an individual may dispense a prescribed amount of nebulizable therapeutic agent solution into the nebulizer inhaler for nebulization. Typically a nebulizable therapeutic agent solution is packaged in the form of single-dose vials, or in the form of a multi-dose bottle having a calibrated dropper.

Ample guidance for obtaining and utilizing a suitable nebulizer inhaler is provided in the literature of the art [refer, for example to O'Callaghan and Barry, 1997. Thorax 52(Suppl 2):S31-S44 S31].

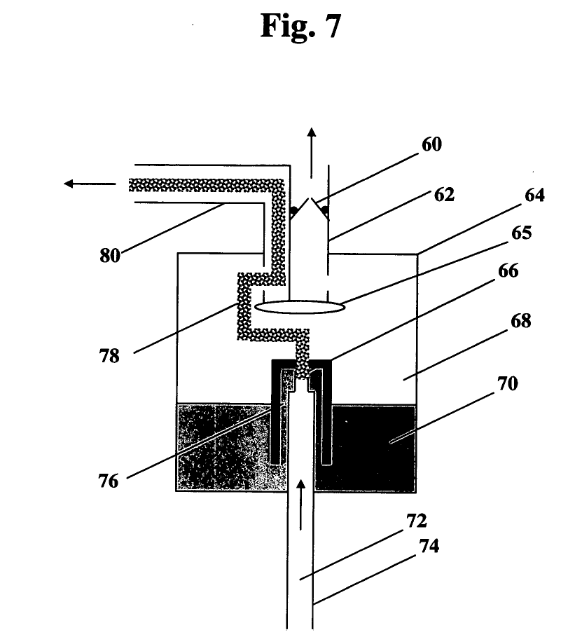

An example of a Venturi-type nebulizer inhaler is shown in FIG. 7. Nebulizer inhaler 64 includes: nebulization chamber 68, fluid 70, which comprises therapeutic agent; pressurized air delivery conduit 74, including pressurized air 72; air-injection hole 66, fluid-feeding conduit 76, baffle 65, aerosol 78, aerosol delivery conduit 80, and expiration conduit 62, including expiration valve 60. For administration of therapeutic agent via the nebulizer inhaler 60, pressurized air 72 is delivered, via air delivery conduit 74, to air-injection hole 66. Upon exit from air-injection hole 66 into fluid-feeding conduit 76, the rapid flow of the exiting pressurized air 72 causes a negative pressure which sucks fluid 70 up fluid-feeding conduit 76 where it encounters exiting pressurized air 72 and becomes aerosol 78. Larger particles of aerosol 78 impact on baffle 65 and walls of nebulization chamber 68, and are returned to fluid 70 for re-nebulization. Aerosol 78 generated, containing therapeutic agent, is delivered, via aerosol delivery conduit 80, to a subject, for example via a respiration mask. Upon expiration by the subject, nebulizer inhaler 64 continues to generate aerosol 78, but the expired air and concomitantly generated aerosol 78 are wasted via expiration conduit 62 via exit valve 60 which allows one-way flow of the wasted gas from nebulization chamber 68.

Dry powder inhalers typically administer a therapeutic agent in the form of a free-flowing powder that can be dispersed in an individual's inhaled air-stream. In order to achieve a free-flowing powder, a therapeutic agent can be formulated with a suitable excipient, such as lactose or starch. A suitable dry powder formulation can be made, for example, by combining dry lactose having a particle size between about 1 micrometer and about 100 microns with micronized particles of a pharmaceutical salt of the active agent and dry-blending. Alternatively, a therapeutic agent can be formulated without excipients. The formulation may be loaded into a dry powder dispenser, or into inhalation cartridges or capsules for use with a dry powder delivery device (for example, Serevent Discus®). Examples of types of DPIs provided commercially include Diskhaler (GlaxoSmithKline, Research Triangle Park, N.C.; see, for example, U.S. Pat. No. 5,035,237); Diskus (GlaxoSmithKline; see, for example, U.S. Pat. No. 6,378,519; Turbuhaler (AstraZeneca, Wilmington, Del.; see, for example, U.S. Pat. No. 4,524,769); and Rotahaler (GlaxoSmithKline; see, for example, U.S. Pat. No. 4,353,365). Suitable types of DPIs are described in U.S. Pat. Nos. 5,239,993, 5,415,162, and 5,715,810 and references cited therein.

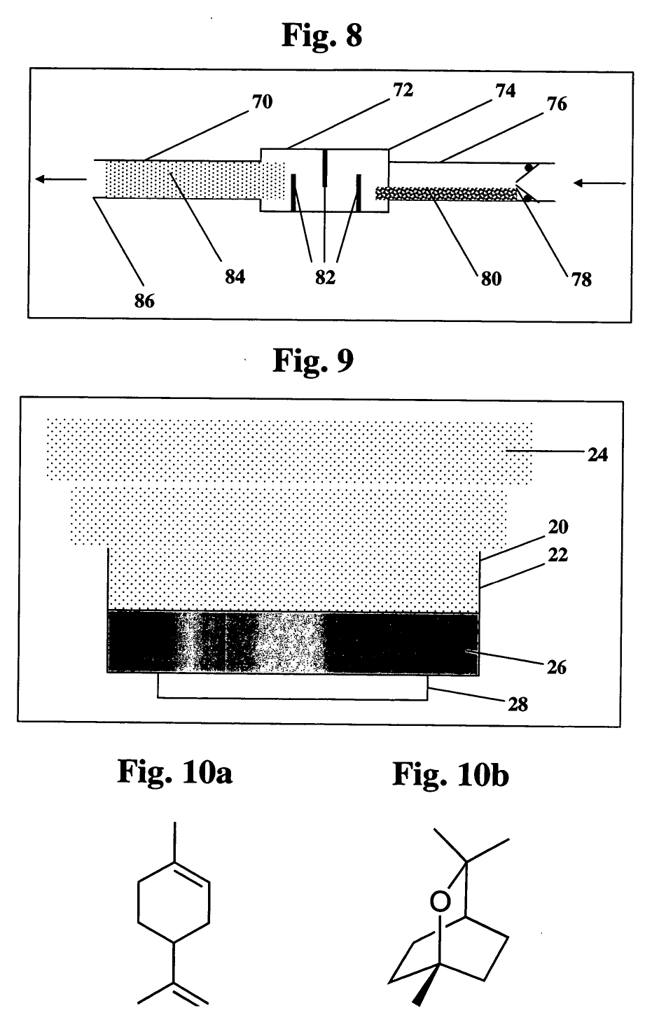

An example of a DPI is shown in FIG. 8. Dry-powder inhaler 74 comprises sediment chamber 76, having air intake valve 78 and sedimented powder 80, which comprises one dose of therapeutic agent; turbulence chamber 72, including turbulence baffles 82 (only three are shown for clarity, any of various numbers thereof be employed, depending on the application and purpose); and suspended powder delivery conduit 70, including suspended powder 84 containing one dose of therapeutic agent, and mouthpiece 86. For administration of a dose of therapeutic agent to a subject via DPI 74, the lips of the subject are sealed around mouthpiece 86, and the subject inhales so as to create an airstream which enters DPI 74 via air intake valve 78, undergoes turbulence in turbulence chamber 72, exits DPI 74 from mouthpiece 86, and enters the respiratory tract of the inhaling subject. The airstream entrains sedimented powder 80 from sediment chamber 76 into turbulence chamber 72 wherein the turbulence generated by turbulence baffles 82 disaggregates particle aggregates in sedimented powder 80 to form suspended powder 84 containing particles of suitable size. Suspended powder 84 containing the therapeutic agent dose is carried in the airstream through suspended powder delivery conduit 70 and mouthpiece 86 to the respiratory tract of the inhaling subject.

The compounds described herein may be administered via an ambient breathing atmosphere generated in habitation volume of the subject, such as in a controlled atmosphere tent, a room, a house, and the like.

A suitable ambient breathing atmosphere may be generated using any of various methods.

Preferably, for administering a substantially volatile therapeutic agent via an ambient breathing atmosphere, the respiratory delivery system is an oil-warmer. An example of an oil-warmer is illustrated in FIG. 9. Oil-warmer 20 is composed of recipient 22 which contains liquid 26, a solution or liquid phase of the at least one compound. Electrical heating element 28, which may be external or immersed in liquid 26, is used to heat liquid 26 so as to produce therapeutic agent vapor 24. The concentration of therapeutic agent in vapor 24 will be proportional to the concentration of therapeutic agent in liquid 26, and the temperature of liquid 26.

It will be appreciated that compounds described herein which are volatile can be suitably administered via an ambient breathing atmosphere in a given habitation volume simply by exposing the habitation volume to a suitable surface area of a solution containing a suitable concentration of the compounds described herein so as to generate a breathing atmosphere containing a vapor of the compounds described herein at a suitable concentration.

A suitable ambient breathing atmosphere may be generated using a humidifier capable of generating a vapor and/or a mist from a solution of the compounds described herein. Various types of commonly employed humidifiers may be employed for such a purpose, including evaporator type humidifiers or atomizer type humidifiers. An evaporator type humidifier forces air over a solution to generate a vapor of the solution. An atomizer type humidifier generates a fine mist that evaporates as it is distributed throughout the house. To air-suspend solution molecules, humidifiers may use a rotating device, like a blade or brush. This may be achieved, in the case of an ultrasonic humidifier, using a disc that oscillates about 1.6 million times per second. Humidifiers can be incorporated into air conditioning systems and may use furnace ducts to distribute moist air throughout a habitation volume such as a house. Alternately, portable humidifiers may be employed. A portable “tabletop” humidifier is typically suitable for a volume about the size of a room. Larger console models can be set up in central locations in a habitation volume to distribute moisture to a larger area than a single room.

A suitable ambient breathing atmosphere may be generated using any of various routinely employed types of atomizers and/or nebulizers, including those which generate a mist of a solution of the compounds using compressed air or ultrasonic dispersion mechanisms.

It will be appreciated by one of ordinary skill in the art, that numerous variations of the respiratory delivery systems described hereinabove are well known to, and routinely employed by, the ordinarily skilled artisan. As such, it will be well within the purview of the ordinarily skilled artisan to adapt the teachings of the present invention towards obtaining and utilizing a suitable inhalation device of the present invention based on essentially any such known respiratory delivery system.

As described hereinabove, the treatment method of the present invention can be used to treat essentially any inflammation-associated medical condition.

Examples of inflammation-associated medical conditions according to all aspects and embodiments of the present invention include, but are not limited to, an inflammatory disease, an idiopathic inflammatory disease, a chronic inflammatory disease, an acute inflammatory disease, an allergic disease, an inflammatory pulmonary disease, an autoimmune disease, an infectious disease, an inflammatory malignant disease, an inflammatory transplantation-related disease, an inflammatory degenerative disease, an inflammatory injury, a disease associated with a hypersensitivity, an inflammatory cardiovascular disease, an inflammatory glandular disease, an inflammatory gastrointestinal disease, an inflammatory cutaneous disease, an inflammatory hepatic disease, an inflammatory neurological disease, an inflammatory musculo-skeletal disease, an inflammatory renal disease, an inflammatory reproductive disease, an inflammatory systemic disease, an inflammatory connective tissue disease, an inflammatory tumor, necrosis, and/or an inflammatory implant-related disease.

Preferably, the inflammation-associated medical condition is an inflammatory pulmonary disease and/or an allergic disease, most preferably both.

Examples of allergic diseases which can be treated via the treatment method of the present invention include asthma, hives, urticaria, a pollen allergy, a dust mite allergy, a venom allergy, a cosmetics allergy, a latex allergy, a chemical allergy, a drug allergy, an insect bite allergy, an animal dander allergy, a stinging plant allergy, a poison ivy allergy, anaphylactic shock, anaphylaxis, and a food allergy.

Examples of inflammatory pulmonary diseases which can be treated via the treatment method of the present invention include asthma, allergic asthma, emphysema, chronic obstructive pulmonary disease and bronchitis.

Examples of hypersensitivities which can be treated via the treatment method of the present invention include Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity, delayed type hypersensitivity, helper T lymphocyte mediated hypersensitivity, cytotoxic T lymphocyte mediated hypersensitivity, TH1 lymphocyte mediated hypersensitivity, and TH2 lymphocyte mediated hypersensitivity.

Examples of inflammatory cardiovascular diseases which can be treated via the treatment method of the present invention include occlusive disease, atherosclerosis, myocardial infarction, thrombosis, Wegener's granulomatosis, Takayasu's arteritis, Kawasaki syndrome, anti-factor VIII autoimmune disease, necrotizing small vessel vasculitis, microscopic polyangiitis, Churg and Strauss syndrome, pauci-immune focal necrotizing glomerulonephritis, crescentic glomerulonephritis, antiphospholipid syndrome, antibody induced heart failure, thrombocytopenic purpura, autoimmune hemolytic anemia, cardiac autoimmunity, Chagas' disease, and anti-helper T lymphocyte autoimmunity.

Examples of inflammatory glandular diseases which can be treated via the treatment method of the present invention include pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome.

Examples of inflammatory gastrointestinal diseases which can be treated via the treatment method of the present invention include colitis, ileitis, Crohn's disease, chronic inflammatory intestinal disease, inflammatory bowel syndrome, chronic inflammatory bowel disease, celiac disease, an ulcer, a skin ulcer, a bed sore, a gastric ulcer, a peptic ulcer, a buccal ulcer, a nasopharyngeal ulcer, an esophageal ulcer, a duodenal ulcer and a gastrointestinal ulcer.

Examples of inflammatory cutaneous diseases which can be treated via the treatment method of the present invention include acne, autoimmune bullous skin disease, pemphigus vulgaris, bullous pemphigoid, pemphigus foliaceus, contact dermatitis and drug eruption.

Examples of inflammatory hepatic diseases which can be treated via the treatment method of the present invention include autoimmune hepatitis, hepatic cirrhosis, and biliary cirrhosis.

Examples of inflammatory neurological diseases which can be treated via the treatment method of the present invention include multiple sclerosis, Alzheimer's disease, Parkinson's disease, myasthenia gravis, motor neuropathy, Guillain-Barre syndrome, autoimmune neuropathy, Lambert-Eaton myasthenic syndrome, paraneoplastic neurological disease, paraneoplastic cerebellar atrophy, non-paraneoplastic stiff man syndrome, progressive cerebellar atrophy, Rasmussen's encephalitis, amyotrophic lateral sclerosis, Sydeham chorea, Gilles de la Tourette syndrome, autoimmune polyendocrinopathy, dysimmune neuropathy, acquired neuromyotonia, arthrogryposis multiplex, optic neuritis, spongiform encephalopathy, migraine, headache, cluster headache, and stiff-man syndrome.

Examples of inflammatory connective tissue diseases which can be treated via the treatment method of the present invention include autoimmune myositis, primary Sjogren's syndrome, smooth muscle autoimmune disease, myositis, tendinitis, a ligament inflammation, chondritis, a joint inflammation, a synovial inflammation, carpal tunnel syndrome, arthritis, rheumatoid arthritis, osteoarthritis, ankylosing spondylitis, a skeletal inflammation, an autoimmune ear disease, and an autoimmune disease of the inner ear.

Examples of inflammatory renal diseases which can be treated via the treatment method of the present invention include autoimmune interstitial nephritis and/or renal cancer.

Examples of inflammatory reproductive diseases which can be treated via the treatment method of the present invention include repeated fetal loss, ovarian cyst, or a menstruation associated disease.

Examples of inflammatory systemic diseases which can be treated via the treatment method of the present invention include systemic lupus erythematosus, systemic sclerosis, septic shock, toxic shock syndrome, and cachexia.

Examples of infectious diseases which can be treated via the treatment method of the present invention include chronic infectious disease, a subacute infectious disease, an acute infectious disease, a viral disease, a bacterial disease, a protozoan disease, a parasitic disease, a fungal disease, a mycoplasma disease, gangrene, sepsis, a prion disease, influenza, tuberculosis, malaria, acquired immunodeficiency syndrome, and severe acute respiratory syndrome.

Examples of inflammatory transplantation-related diseases which can be treated via the treatment method of the present invention include graft rejection, chronic graft rejection, subacute graft rejection, acute graft rejection hyperacute graft rejection, and graft versus host disease.

Examples of implants include a prosthetic implant, a breast implant, a silicone implant, a dental implant, a penile implant, a cardiac implant, an artificial joint, a bone fracture repair device, a bone replacement implant, a drug delivery implant, a catheter, a pacemaker, an artificial heart, an artificial heart valve, a drug release implant, an electrode, and a respirator tube.

the inflammatory tumor is selected from the group consisting of a malignant tumor, a benign tumor, a solid tumor, a metastatic tumor and a non-solid tumor.

Examples of inflammatory injuries which can be treated via the treatment method of the present invention include abrasion, a bruise, a cut, a puncture wound, a laceration, an impact wound, a concussion, a contusion, a thermal burn, frostbite, a chemical burn, a sunburn, a desiccation, a radiation burn, a radioactivity burn, a smoke inhalation, a torn muscle, a pulled muscle, a torn tendon, a pulled tendon, a pulled ligament, a torn ligament, a hyperextension, a torn cartilage, a bone fracture, a pinched nerve and a gunshot wound.

As is described in Example 1 of the Examples section which follows, the treatment method of the present invention can be used to significantly reduce in inflamed tissues peribronchial and perivascular infiltration of inflammatory cells such as lymphocytes and granulocytes, in particular neutrophils. It will be appreciated by one ordinarily versed in the art that since such lymphocytic/granulocytic tissue infiltration mediates pathogenic inflammation in essentially all inflammation-associated medical conditions, by virtue of enabling optimal reduction of such infiltration, the treatment method of the present invention can be used for optimally treating essentially any such medical condition.

The treatment method of the present invention can be used for treating a medical condition associated with inflammation mediated by any of various exogenous oxidants, such as the ozone component of air pollution inhaled by the subject, and/or mediated by any of various biological oxidants generated in-vivo in tissues/fluids of the subject which are affected by the inflammation associated with the disease. As described above, such biological oxidants include ozone produced by antibodies and activated leukocytes such as B-cells and neutrophils, and superoxide anion produced by activated leukocytes such as neutrophils.

As described above, oxidants, such as ozone, hydrogen peroxide, superoxide, hypochlorous acid and/or hydroxyl radicals are involved in mediating numerous inflammatory processes, including: degranulation of mast cells; cellular synthesis and release of proinflammatory mediators such as cytokines, histamine, leukotrienes, and antibodies; adhesion, migration and aggregation of lymphocytes, such as B- and T-cells, and granulocytes, such as eosinophils and neutrophils; and increase in vascular permeability. As is further described hereinabove, the compounds described herein can be used for optimally inhibiting the formation and/or activity of such oxidants. As such the compounds described herein can be used for optimally inhibiting all such inflammatory processes.

Thus, according to further embodiments the present invention provides methods of optimally inhibiting inflammatory processes including: degranulation of mast cells; cellular synthesis and/or release of proinflammatory mediators such as cytokines, histamine, leukotrienes, and/or antibodies; adhesion, migration and/or aggregation of lymphocytes, such as B- and/or T-cells, and/or granulocytes, such as eosinophils and/or neutrophils; and/or increase in vascular permeability.

Thus, according to another aspect of the present invention there is provided a method of qualifying a presence of an oxidant in a sample. The method is effected by contacting the sample with a medium containing a compound in a mesophase state, which compound being susceptible to a degradation induced by the oxidant; and evaluating a capacity of the sample to induce degradation of the compound.

The underlying concept of this and the following aspects and methods of the present invention is based on the capability of some of the compounds of the present invention, described hereinabove, to readily react stoichiometrically with oxidants and to undergo degradation as a result of such reaction, as is described hereinabove (see, for example, FIG. 3). Hence, by utilizing a specifically characterized compound or a medium containing the compound, the compound's degradation as a result of the stoichiometric reaction with the oxidant can be easily monitored, thus enabling qualification and/or quantification of the reaction with respect to the oxidant and/or the compound.

The preferred modes of operation of this and the following aspects of the present invention utilize a compound in a mesophase state. A mesophase state is characterized by specific, measurable physical properties and is further susceptible to alteration as a function of minor changes in the medium, e.g. the compound's concentration. Detecting a degradation of a compound in a mesophase state can be therefore easily performed.

Thus, in a preferred embodiment of this and the following aspects of the present invention, evaluating a capacity of the sample to induce degradation of the compound is effected by measuring and/or characterizing a physical property of the medium. The physical property can be a phase of matter, such as, for example, a liquid crystalline phase, a solid phase and a liquid phase. Changes in the phase of matter of the compound in the medium can therefore provide a measure of its degradation.

Alternatively and preferably, the physical property is an optical birefringence. Compounds in a mesophase state are characterized by specific optical properties such as birefringence. Measuring an optical property in general and optical birefringence in particular is highly advantageous as such measurements are highly sensitive, precise and easy to perform.

Representative examples of compounds according to the present invention, which can be used in this and the following methods of the present invention, are chromone derivatives in general (depicted, for example, in Formula II hereinabove), and cromolyn sodium in particular (see, FIG. 4e).

As is described hereinabove, chromone derivatives readily react with various oxidants, so as to thereby undergo degradation. Such oxidants particularly include ozone, as well as reactive oxygen species, hydrogen peroxide, superoxide ions, superoxide radicals, hydroxyl radicals and hypohalous acids. Chromone derivatives can therefore be used to qualify the presence of any one of these oxidants, and particularly ozone.

As is described in detail in the Examples section that follows, cromolyn sodium (CS) forms a lyotropic liquid crystalline mesophase at concentrations higher than approximately 7% (w/w) in an aqueous medium. Thus, reduction of such a concentration, as a result of a degradation of CS by reaction thereof with an oxidant, would result in phase change of the aqueous medium from liquid crystalline mesophase to liquid phase.

The method, according to this and the following aspects of the present invention is therefore effected by contacting the sample with a medium (e.g. an aqueous solution) containing CS in a mesophase state, and evaluating, for example, the optical birefringence changes of the medium as a result of this contacting, to thereby evaluate the degradation of CS by the oxidant.

The mesophase can be a lyotropic mesophase. Alternatively, the mesophase can be a cholesteric phase, for example, a lyotropic liquid crystal, such as CS, contaminated with cholesterol or any other unsaturated asymmetric compound that can readily react with an oxidant. Upon contacting the sample, the unsaturated compounds undergo degradation, which results in changing the mesophase from cholesteric to nematic, a change that is significantly visible by optical methods.

As the method described hereinabove is based on changes in the phase of a matter, which, as is known in the art, are highly sensitive to minor changes in the concentration of the matter, this and the following methods can be used to detect minute amounts of an oxidant in a sample.

As is described hereinbelow, according to one embodiment the method of this aspect of the present invention can be practiced by using a sample which includes one or more cells so as to detect oxidant production by such cells. As is described hereinbelow, such cells may include mast cells, macrophages, neutrophils, basophils, eosinophils and the like. Due to its high sensitivity, the method can be used for qualifying the presence of an oxidant produced by a single cell.

While reducing the present invention to practice, the present inventors have succeeded in using the method of qualifying the presence of the oxidant to detect ozone production by activated human neutrophils, as is described in Example 3 of the Examples section which follows. It will be appreciated that since inflammation-associated medical conditions, such as those described above, are characterized by cellular production of oxidants, such as ozone and/or superoxide anion, the method of qualifying the presence of the oxidant can be used for diagnosing such a medical condition.

Thus, according to the present invention, there is provided a method of diagnosing a medical condition associated with inflammation in a subject. The method is effected by contacting at least one cell which is derived from the subject and is capable of producing an oxidant, with a medium containing a compound in a mesophase state, the compound being susceptible to degradation induced by the oxidant; and evaluating a capacity of the at least one cell to induce degradation of the compound.

The method can be used for diagnosing in a subject any of various inflammation-associated medical conditions, as described hereinabove. Preferably, the method is used for diagnosing an inflammatory pulmonary disease or an allergic disease, most preferably an allergic/inflammatory pulmonary disease such as asthma. The method can be used for diagnosing the medical condition in essentially any homeothermic vertebrate, preferably a mammal, and most preferably a human.

The type of cells used are preferably immune cells, more preferably activated immune cells. Preferably, the immune cell type is granulocytic, most preferably of neutrophil type. As described above, activated neutrophils produce oxidants such as ozone and superoxide anion which are involved in triggering/maintenance of inflammation in numerous inflammation-associated medical conditions. Alternately, the immune cell type may be of B-cell type. As is also described above, antibodies, which are produced by B-cells, or which are surface-displayed by B-cells in the form of membranal B-cell receptors, are involved in proinflammatory ozone production. The method may be performed using any of various types of immune cells, so long as oxidant production by such a cell type is involved in the inflammation associated with the medical condition.

The cell types used are preferably derived from a mammal, most preferably from a human.

Preferably, a population of about one million cells in a volume of medium of about 1 mL is used to practice the diagnosis method. Alternately, it is envisaged by the present invention to perform the diagnosis method using any number of cells, including using a single cell.

Preferably, when using a plurality of cells, the cells preparation is purified to greater than 85% purity, more preferably to greater than 90% purity, more preferably to greater than 91% purity, more preferably to greater than 92% purity, more preferably to greater than 93% purity, and most preferably to greater than 94% purity. Alternately, non-purified cell populations may be employed. Numerous methods are known to the ordinarily skilled artisan for purifying a desired cell population. Neutrophils may be conveniently substantially purified to 95% purity as described in Example 3 of the Examples section below.

Preferably, the cells are isolated from the subject prior to being contacted with the medium.

Preferably, to enable diagnosis of the medical condition, the cells used are isolated from a tissue or body fluid affected by the inflammation associated with the medical condition. For example, for diagnosing an inflammatory pulmonary disease such as asthma, a suitable source from which to obtain cells for used in the diagnosis method would be from bronchoalveolar lavage fluid, pulmonary phlegm, airway biopsy samples, etc. Similarly, to diagnose an inflammation-associated medical condition affecting the joints, such as rheumatoid arthritis, synovial fluid of the organism will be a preferred source for obtaining suitable cells. A suitable source from which to obtain the cells will be evident to the ordinarily skilled artisan.

The cells may be derived from essentially any suitable body fluid of the subject, including synovial fluid, cerebrospinal fluid, lymph, gastrointestinal secretions, saliva, urine, feces, or lacrymal secretion. The cells may be derived, as appropriate, from any of various tissues of the organism, for example, via a tissue biopsy. Peripheral blood of the subject may be a preferred source, depending on the application and purpose. As is described in Example 3 of the Examples section below, neutrophils may be conveniently isolated from a body fluid such as peripheral blood according to the density gradient centrifugation protocol set forth therein (based on Markert et al., 1984. Methods Enzymol. 105:358). Peripheral blood immune cells of a desired type may also be isolated from blood via leukopheresis. The cells used may represent a population of different cell types, such as whole peripheral blood mononuclear cells (PBMCs), or it may be a substantially purified cell population. Immune cells displaying any desired surface marker or combination thereof, such as neutrophils or B-cells, may be effectively isolated from a cell suspension, such as a PBMC suspension, by various common art techniques, such as fluorescence activated cell sorting (FACS), magnetic cell sorting (MACS), etc.

One of ordinary skill in the art, such as a physician, preferably a physician specialized in the medical condition will possess the necessary expertise for isolating suitable cells from a subject having the medical condition so as to enable diagnosis of the medical condition in the subject.

It will be appreciated by one of ordinary skill in the art that by virtue of enabling detection of proinflammatory oxidant production by an activated immune cell type such as an activated neutrophil, the method of the present invention can be used for identifying a candidate compound for treating a medical condition associated with inflammation mediated by such an oxidant

It will be further appreciated by the ordinarily skilled artisan that the diagnosis method can be performed using high-throughput methods well known to the ordinarily skilled artisan so as to facilitate rapid and consistent analysis of large numbers of samples.

Thus, according to the present invention there is provided a method of identifying a candidate compound for treating a medical condition associated with inflammation. The method is effected by exposing the medium containing the compound in a mesophase state and a test compound to the oxidant, and evaluating a capacity of the test compound to regulate the degradation of the compound in the mesophase state.

The method can be used to identify a candidate compound optimal for treating essentially any medical condition associated with inflammation, such as those described hereinabove. Preferably, the method is used for identifying a candidate compound for treating an inflammatory pulmonary disease or an allergic disease, most preferably an allergic/inflammatory pulmonary disease such as asthma. The method may be used for identifying a candidate compound for treating a medical condition in essentially any homeothermic vertebrate, preferably in a mammal, and most preferably in a human.

According to a preferred embodiment, exposing the medium and the test compound to the oxidant is preferably effected by exposing the medium and the test compound to the cells.

Depending on the application and purpose, suitable cells for practicing the candidate compound identification method may include primary cells and/or cultured cell lines.

Suitable primary cells, and methods of isolation thereof, for practicing the candidate compound identification method are as described hereinabove with respect to the cells used for practicing the diagnosis method of the present invention.

Various suitable cultured cell lines, such as cultured neutrophil-lineage cells (e.g. HL-60 cells, ATCC No. CCL-240), which are available to the ordinarily skilled artisan may be employed to practice the candidate compound identification method. Cultured cell lines are advantageous in that they are infinitely propagatable and hence do not require isolation from primary sources, and retain greater phenotypic homogeneity compared to isolates from different primary sources, such as from different human donors.

Preferably, when using immune type cells, such as neutrophils, for practicing the candidate compound identification method, the cells are activated. It will be appreciated that suitable activation of suitable immune cell types will result in production of proinflammatory oxidants, such as ozone, by such cells.

Activation of immune cells of various types can be effected using techniques routinely employed by one of ordinary skill in the art. Preferably, obtaining an activated immune cell for practicing the candidate compound identification method of the present invention is effected by treating the immune cell with phorbol myristate acetate (PMA) as described in Example 3 of the Examples section which follows.

Preferably, the candidate compound identification method is performed using a population of about one million cells in a volume of medium of about 1 mL. Alternately, the method may be performed using a lower cell density.

Preferably, a population of a cell type used for practicing the candidate compound identification method is purified to greater than 85% purity, more preferably to greater than 90% purity, more preferably to greater than 91% purity, more preferably to greater than 92% purity, more preferably to greater than 93% purity, and most preferably to greater than 94% purity. Alternately, non-purified cell populations may be employed. Numerous methods are known to the ordinarily skilled artisan for purifying a desired cell population.

In general performing the candidate compound identification method of the present invention is preferably performed according to the guidelines provided hereinabove and in Example 3 of the Examples section which follows. The ordinarily skilled artisan will possess the necessary expertise for adapting the teachings of the present invention for performing the candidate compound identification method of the present invention in any of various ways.

The candidate compound identification method may be practiced using a test compound of any of various types, by evaluating and comparing the capacity of the test compound to react with the oxidant with the same capacity of a compound in a mesophase state. Based on the guidelines described hereinabove, the presence of a test compound that is capable of reacting with the oxidant more rapidly than the compound in the mesophase state would result in no significant measurable change in the medium containing the compound in the mesophase state, thus indicating such a test compound as a favorable candidate compound for treating a medical condition associated with inflammation. The presence of a test compound that is not capable of reacting with the oxidant, or that reacts with the oxidant at a slower rate than the compound in the mesophase state, would result in a measurable change in the medium containing the compound in the mesophase state, thus indicating such a test compound as an unfavorable candidate compound for treating a medical condition associated with inflammation.

As is described hereinabove, a preferred compound in a mesophase state which can be advantageously used in the method according to this aspect of the present invention is cromolyn sodium (CS). As CS is known as an efficient asthma medication, according to the present invention, a test compound which is more reactive than CS toward degradation by an oxidant will constitute a candidate compound which is potentially superior to CS for treatment of an inflammatory medical condition such as asthma.

It will be appreciated by the ordinarily skilled artisan that the candidate compound identification method of the present invention can be performed using high-throughput methods well known to the ordinarily skilled artisan so as to facilitate rapid and consistent analysis of large numbers of samples.

As described hereinabove, the method of identifying the candidate compound of the present invention may be effected using any of various types of candidate compounds.

Test compounds of any of various types may be obtained from a commercial chemical library such as, for example, one held by a large chemical company such as Merck, Glaxo Welcome, Bristol Meyers Squib, Monsanto/Searle, Eli Lilly, Novartis, Pharmacia UpJohn, and the like. Test compounds of any of various suitable types may also be ordered via the World Wide Web (Internet) via companies such as Chemcyclopedia (http://www.mediabrains.com/client/chemcyclop/BG1/search.asp). Alternatively, test compounds of any of various suitable types may be synthesized de novo using standard chemical and/or biological synthesis techniques. Ample guidance for synthesis of molecules suitable for use as test compounds of any of various suitable types is provided in the literature of the art. For biological synthesis of molecules, such as polypeptides and nucleic acids, refer, for example to: Sambrook et al., infra; and associated references in the Examples section which follows. For guidance regarding chemical synthesis of molecules, refer, for example to the extensive guidelines provided by The American Chemical Society (http://www.chemistry.org/portal/Chemistry). One of ordinary skill in the art, such as, for example, a chemist, will possess the required expertise for chemical synthesis of suitable test compounds.

As used herein, the term “peptide” includes native peptides (either degradation products, synthetically synthesized peptides or recombinant peptides) and peptidomimetics (typically, synthetically synthesized peptides), such as peptoids and semipeptoids which are peptide analogs, which may have, for example, modifications rendering the peptides more stable while in a body or more capable of penetrating into target cells. Such modifications include, but are not limited to N terminus modification, C terminus modification, peptide bond modification, including, but not limited to, CH₂—NH, CH₂—S, CH₂—S═O, O═C—NH, CH₂—O, CH₂—CH₂, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992).

Peptide bonds (—CO—NH—) within the peptide may be substituted, for example, by N-methylated bonds (—N(CH₃)—CO—), ester bonds (—C(R)H—C—O—O—C(R)—N—), ketomethylen bonds (—CO—CH₂—), α-aza bonds (—NH—N(R)—CO—), wherein R is any alkyl, e.g., methyl, carba bonds (—CH₂—NH—), hydroxyethylene bonds (—CH(OH)—CH₂—), thioamide bonds (—CS—NH—), olefinic double bonds (—CH═CH—), retro amide bonds (—NH—CO—), peptide derivatives (—N(R)—CH₂—CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables 1 and 2 below list naturally occurring amino acids (Table 1) and non-conventional or modified amino acids (Table 2) which can be used with the present invention.

TABLE 1
Naturally occurring amino acids.

| | Three-Letter | One-letter |
|---|---|---|
| Amino Acid | Code | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Iie | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid | Xaa | X |
| as above | | |

TABLE 2
Non-conventional or modified amino acids.

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl) glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nile | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomo phenylalanine | Mhphe |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | mser | L-α-methylthreonine | Mthr |
| L-α-methylvaline | Mtrp | L-α-methyltyrosine | Mtyr |
| L-α-methylleucine | Mval Nnbhm | L-N-methylhomophenylalanine | Nmhphe |
| N-(N-(2,2-diphenylethyl) | | N-(N-(3,3-diphenylpropyl) | |
| carbamylmethyl-glycine | Nnbhm | carbamylmethyl(1)glycine | Nnbhe |
| 1-carboxy-1-(2,2-diphenyl | Nmbc | | |
| ethylamino)cyclopropane | | | |

The peptides of the present invention can be utilized in a linear or cyclic form.

A peptide can be either synthesized in a cyclic form, or configured so as to assume a cyclic structure under suitable conditions.

For example, a peptide according to the teachings of the present invention can include at least two cysteine residues flanking the core peptide sequence. In this case, cyclization can be generated via formation of S—S bonds between the two Cys residues. Side-chain to side chain cyclization can also be generated via formation of an interaction bond of the formula —(—CH2—)ₙ—S—CH—₂—C—, wherein n=1 or 2, which is possible, for example, through incorporation of Cys or homoCys and reaction of its free SH group with, e.g., bromoacetylated Lys, Orn, Dab or Dap. Furthermore, cyclization can be obtained, for example, through amide bond formation, e.g., by incorporating Glu, Asp, Lys, Om, di-amino butyric (Dab) acid, di-aminopropionic (Dap) acid at various positions in the chain (—CO—NH or —NH—CO bonds). Backbone to backbone cyclization can also be obtained through incorporation of modified amino acids of the formulas H—N((CH₂)n-COOH)—C(R)H—COOH or H—N((CH₂)n-COOH)—C(R)H—NH₂, wherein n=1-4, and further wherein R is any natural or non-natural side chain of an amino acid.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology,” John Wiley and Sons, Baltimore, Md. (1989); Perbal, “A Practical Guide to Molecular Cloning,” John Wiley & Sons, New York (1988); Watson et al., “Recombinant DNA,” Scientific American Books, New York; Birren et al. (eds) “Genome Analysis: A Laboratory Manual Series,” Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook,” Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology,” W.H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications,” Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### Example 1

### Optimal Asthma Treatment/Prevention Using the Ozone Scavenger D-Limonene

Background: Inflammation-associated medical conditions, such as asthma, include numerous highly debilitating and/or lethal medical conditions for which no optimal therapy exists. While conceiving the present invention, the present inventors theorized that proinflammatory oxidants, such as ozone, may be involved in the inflammatory pathogenesis of such medical conditions. Hence, the present inventors predicted that methods of inhibiting or preventing oxidation at sites of inflammation by proinflammatory oxidants, such as ozone, could be used as a general method to treat inflammatory diseases, such as asthma. While various methods have been proposed in the prior art for preventing oxidation by proinflammatory oxidants so as to treat inflammation-associated medical conditions, these have been highly suboptimal for various reasons, as described above. As described below, while reducing the present invention to practice, a method of optimally treating inflammatory diseases, such as asthma, by inhibiting oxidation by proinflammatory oxidants, such as ozone, was unexpectedly uncovered, thereby overcoming the limitations of the prior art, as described below.

Materials and Methods:

Animals: Four-week-old Brown Norway (BN) male rats, obtained from Harlan Inc., USA, were used in this study. The rats were maintained on OVA-free diets. All experimental animals used in this study were under a protocol approved by the Institutional Animal Care and Use Committee of the Hadassah Medical School of the Hebrew University of Jerusalem).

Asthma treatment model I: In a preliminary experiment, 15 ml of limonene was placed in a 50 ml beaker over a wire cage containing two brown Norway rats. The rats were pre-induced to develop an asthmatic condition upon challenge with ovalbumin, as is detailed hereinbelow, and were allowed to eat and drink at will. The cage was covered with a paper filter and was left as such for 20 hours. A control group of three rats was placed in a separate covered cage not containing limonene, for the same time period. Thereafter, rats of both groups were challenged with an aerosol of an ovalbumin and Penh values of all rats were measured, as is detailed hereinbelow.

Asthma treatment model II: Asthma was induced on day 0 in three experimental groups of 10 animals each, by sensitization with ovalbumin (OVA) and aluminum hydroxide, as previously described (Du et al., 1992. Am. Rev. Resp. Dis. 146:1037-1041). Sensitization to the model antigen ovalbumin (OVA) was carried out by subcutaneous injection of 1 mg OVA (Sigma, St. Louis, Mo.) and 200 mg of aluminum hydroxide (AlumInject; Pierce Chemical, Rockford, Ill.) in 0.9 percent (w/w) saline in a total volume of 1 ml, and intraperitoneal injection of 1 ml saline containing 6 billion heat killed Bordetella pertussis cells (Pasteur Marieux, France). A fourth group of 10 animals was not sensitized and was used as the naive control group. The three groups of sensitized rats as well as the group of naive animals were challenged every other day from day 14 until day 21 with repeated 10 minute long inhalations of OVA using an ultrasonic nebulizer inhaler (LS 230 Systam Villeneuve Sur Lot, France). Throughout the experiments, the rats were kept unrestrained in a 20 liters box. To assess the anti-asthma effects of limonene, two groups of sensitized rats were continuously subjected during the OVA challenge period (from day 14 to day 21) to a breathing atmosphere containing an average concentration of 125 ppm of d-limonene (limonene; Aldrich, Catalog No. W26330-3, FIG. 10a) or eucalyptol (Aldrich, Catalog No. W24650-6, FIG. 10b). The atmosphere containing limonene or eucalyptol was generated using a continuously operated electric oil warmer placed inside the cage loaded with the respective compound.

Assessment of treatment. Bronchoconstriction was measured via a modification of previously described non-invasive method (Hamelmann et al., 1997. Am. J. Respir. Crit. Care Med. 156:766-775) using a barometric plethysmography, and expressed as enhanced pause (Penh), a calculated dimensionless value that correlates with measurement of airway resistance, impedance, and intrapleural pressure calculated according to: Penh=(PEF/PIF)×((Te−Tr)/Tr), where PEP=peak expiratory flow, PIP=peak inspiratory flow, Te=expiratory time, Tr=relaxation time (time of the pressure decay to 36 percent of total box pressure during expiration). Unrestrained conscious rats were placed in a whole-body plethysmograph (Buxco Electronics Inc., Troy, N.Y., USA) connected to a pneumotach (EMKA Technologies, Type 0000), which was connected to a 10 ml bottle, and connected to a preamplifier (Buxco Electronics, model MAX2270). Analog signals from the amplifier were converted to a digital signal by an AD card and analyzed using a software package (Data Translation, Texas, U.S.A.) to calculate the respiratory rate, Te, Ti, and Penh). Twenty hours (model I) or 21 days (model II) post induction, Penh was measured 5 minutes following allergen challenge. Statistical analytical treatment of the data was performed using EXCEL software (Microsoft Corp., U.S.A.). Analysis of variance was performed on the interval data (Penh) and the Kruskal Wallis Test was used for the pathology scores.

In model II, all rats were anaesthetized on day 21 with pentothal and sacrificed by bleeding from the abdominal aorta. Lungs were removed and inflated with 4 percent buffered formaldehyde overlayed with 20 cm water. The lungs were sliced longitudinally and embedded in paraffin. Histologic sections 5 microns thick were cut and stained with hematoxylin and eosin. The morphological changes were evaluated by light microscopy by a pathologist who was blinded to the treatment groups. The intensity of the peribronchiolar and perivascular cellular infiltration was assessed semi-quantitatively on a 0-3 scale as follows: 0=no or practically no inflammatory cells; I=a narrow rim of inflammatory cells surrounding most of the bronchioles/blood vessels, best visualized under high power; 2=a rim of inflammatory cells 3-4 cells thick, surrounding most of the bronchioles/blood vessels; 3=a prominent rim of inflammatory cells, 5 or more cells thick, surrounding most of the bronchioles/blood vessels. Bronchiolar constriction was also assessed on a semi-quantitative scale ranging over 0-3 grades (0=none; 1=mild; 2=moderate; 3=marked). The histologic sections included bronchioles (defined as airways lined by mucosa and lacking cartilage) and distal airways (alveolar ducts and sacs). The features examined included peribronchiolar and perivascular inflammation, morphological evidence of bronchoconstriction (papillary infolding of the bronchiolar mucosa) and granulomatous response in the pulmonary parenchyma.

Experimental Results:

Asthma treatment model I: The ability of limonene, a monoterpene having two double bonds, to prevent asthma onset in sensitized-rats was preliminarily evaluated, by subjecting animals to ovalbumin challenge in the presence (experimental group) or absence (control group) of limonene and examining the Penh values of both groups.

After 20 hours in a covered cage, the sensitized rats of both the control and experimental groups exhibited similar baseline Penh values, indicating that rats of both groups had unchanged pulmonary activity at this stage of the experiment. Upon challenge with ovalbumin aerosol, the control group developed severe asthmatic condition, as reflected by the measured Penh values of 16%, 22% and 59%. In contrast, the rats of the experimental group were not substantially affected by the challenge, as reflected by the measured Penh values of 2% and 3%.

Asthma treatment model II: The two closely related monoterpenes limonene and eucalyptol were examined for their ability to prevent asthma onset in sensitized-rats. Both limonene and eucalyptol share the same boiling point of 176 degrees centigrade, and have a similar low molecular weight (136 and 154 Da, respectively). The two double bonds in limonene render the molecule a highly reactive ozone scavenger, while eucalyptol, being a saturated compound, is totally inert towards ozone. The chemical structures of limonene and eucalyptol are shown side by side in FIG. 10a-b.

In order to assess the anti asthma properties of limonene, ovalbumin sensitized animals were subjected to ovalbumin challenge while being treated with limonene or eucalyptol inhalation, and examined with respect to Penh values and various pathological parameters, as described under Materials and Methods, above. As a negative control, non-sensitized animals were subjected to ovalbumin challenge.

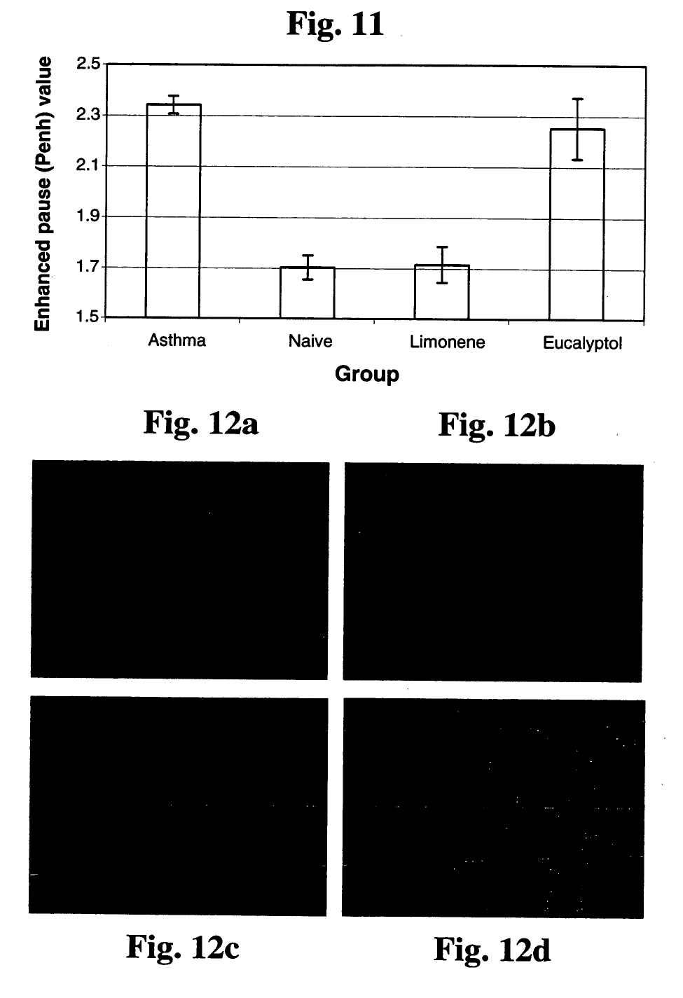

The Penh values (FIG. 11) of the sensitized group (asthma) were found to be as high as those of the eucalyptol-treated sensitized rats (2.342±0.004 and 2.25±0.12, respectively). In contrast, the naive and the limonene-treated sensitized rats exhibited significantly lower Penh values (1.70±0.05 and 1.71±0.07, respectively, p≦0.05). These observations clearly show that limonene inhalation significantly prevented bronchial obstruction while eucalyptol inhalation did not cause any change of bronchoconstriction in the sensitized rats.

Histological examination of lung sections using H&E staining from the four experimental groups revealed a highly protective effect conferred by limonene as opposed to eucalyptol (FIGS. 12a-d). Peribronchiolar and perivascular inflammatory infiltrates were found to be composed principally of eosinophils and lymphocytes, with the eosinophils being disposed in a circumferential manner around the bronchioles and vessels, and the lymphocytes being either similarly disposed or arranged in primary follicles (lymphoid follicles without germinal centers). In addition, multinucleated giant cells and granulomas were seen in the pulmonary parenchyma in all four groups of rats. Polarizing microscopy did not reveal foreign material. Such appearance of granulomatous lesions in this asthma model has been previously described (Michielsen, S. et al., 2002. Archives of Toxicology 76:236-247).

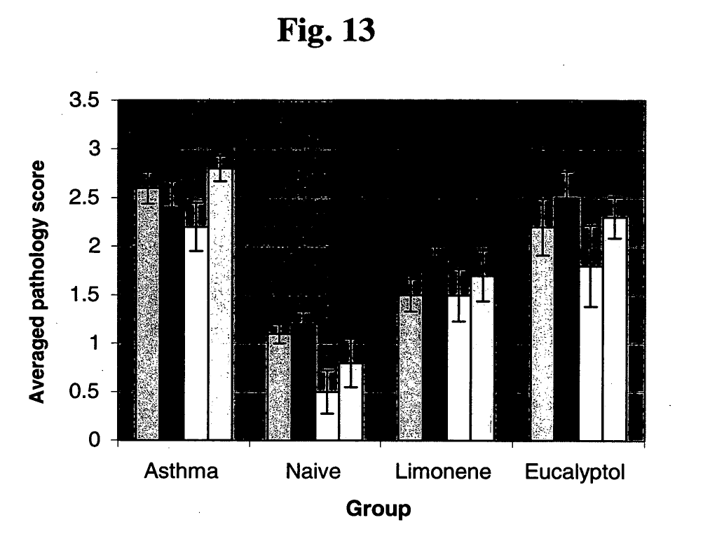

Results obtained upon examination of various pathological parameters follow and confirm the general trend that was exhibited by the pulmonary function test, and histological examination (FIG. 13). Yet, there are subtle variations that may lead to further conclusions. Since the essential oils were administered by inhalation, it is expected that the bronchi would be influenced much more than the parenchyma. Indeed, as can be seen from representative H&E histological examination of lung sections specific from the four experimental groups, OVA sensitized, OVA challenged rats treated with limonene showed significant reduction of peribronchial inflammatory cell infiltration in comparison with the eucalyptol-treated group (limonene 1.5±0.17, eucalyptol 2.2±0.29, p<0.05) and, to a lesser extent, perivascular inflammatory cell infiltration (limonene 1.9±0.1, eucalyptol 2.5±0.27, p<0.05). The morphological evidence of bronchoconstriction followed the same trend (limonene 1.7±0.26, eucalyptol 2.3±0.21, p<0.05). In contrast, the effect on the granulomas, which are located further away from the bronchi, was found to be insignificant (limonene 1.5±0.27, eucalyptol 1.8±0.42, p=0.43).

Conclusion: The above described pulmonary function tests, histological examination of leukocytic infiltration, and pathological assessment of various key parameters convincingly demonstrate that an inflammation-associated medical condition, such as asthma, can be optimally prevented and treated using an ozone scavenger such as limonene, according to the presently described method.

### Example 2

### Effective Treatment of Asthma in Humans Using the Ozone-Scavenging Compound Limonene

Background: As described above, while reducing the present invention to practice, it was demonstrated, in a highly realistic mammalian disease model, that compounds, such as limonene, capable of scavenging proinflammatory oxidants, such as the ozone, could be used to effectively treat inflammation-associated medical conditions, such as asthma. As described below, while further reducing the present invention to practice, the present inventors demonstrated the capacity of compounds such as limonene to be effective in treating medical conditions such as asthma in human subjects suffering from this condition.

Materials and Methods:

Patients: Eleven patients were recruited from children to the pediatric pulmonary clinic, Rambam Medical Center, Haifa. Inclusion criteria: age: 6-11 years; diagnosis of asthma by ATS criteria; ability to perform spirometry consistently; asthma severity classified as “mild persistent” as per the GINA workshop report; no anti-inflammatory medication, including antihistamines, taken for at least 2 weeks prior to enrollment. Exclusion criteria: presence of other chronic conditions; emergency room visit in the past 3 months; respiratory infection in the past month; steroids or other anti-inflammatory medication taken within 2 weeks prior to treatment; and bronchodilators, theophylline preparations or monteleukast taken in the 24 hours prior to spirometry.

Study design: A double-blind cross-over study format was used to evaluate the effect of monoterpenes on asthma. The study included four phases, each lasting two weeks. Following enrollment, baseline spirometry was performed and inclusion/exclusion was verified. The patients then filled in a diary card for two weeks. At the end of these two weeks, spirometry and the first adenosine 5′-monophosphate (adenosine) bronchial provocation test (BPT) was performed (Phase I). Each subject received an electrical vaporizer based on a commercial electrical air freshener (Air Wick®/Wizard®) modified to vaporize a liquid containing a test compound by heating. The vaporizer was operated at night in the patient's room so as to generate a breathing atmosphere containing about 80 ppm of either the test compound d-limonene, a volatile ozone-scavenging monoterpene oil, or as the negative control test compound, cineole, a non ozone-scavenging oil by virtue of lacking a double bond. These oils have identical color and volatility, the latter by virtue of their sharing identical boiling points and vapor pressures. After two weeks of use, the diary card and the vaporizers were collected and spirometry and the second adenosine BPT were performed (Phase II). Following a two-week washout period, during which patients continued to fulfill diary cards (Phase III), another vaporizer containing the second oil was provided with a fourth diary card. Two weeks later spirometry and the fourth adenosine BPT were performed (Phase IV).

Symptom scoring: The subjects completed a standard asthma daily symptom diary recording day cough, night cough, exercise tolerance, and use of bronchodilator medicine. Each category is graded as none (‘0’), mild (‘1’), or significant (‘2’). Total scores for two-week periods, corresponding to the four study phases is tallied and compared. In addition, asthma exacerbations, use of rescue medications, doctor visits and presence of other illnesses or symptoms are noted in the symptom diary.

Adenosine bronchial provocation test (BPT): Bronchial provocation tests were performed by inhalation of nebulized adenosine 5′-monophosphate (adenosine; Sigma chemical company, St. Louis, Mo., USA) solutions in doubling doses. Fresh solutions of adenosine were prepared at doses ranging in double increments from a minimum of 0.39 mg/mL to a maximum of 200 mg/mL. Two milliliters of adenosine solution were nebulized using a nebulizer (Hudson Up-Draft Nebulizer, MMD 1.5 microns [85%]) at a flow rate of 5 liters/minute. The patient inhaled the solution for 2 minutes through a mouthpiece, after which spirometry was performed using a spirometer (Microloop II Spirometer, Micro Medical Ltd., Rochester, Kent, England) was performed. The best of three efforts were recorded. The provocation study was continued at 5-minute intervals until the FEV_{1.0} dropped 20% below baseline level (PC₂₀), or until a dose of 200 mg/mL was reached (negative result).

Statistics: ANOVA are used to determine the difference of each variable at the three phases of the study. Comparison of the variables (PC₂₀, FEV_{1.0}, FVC, and Symptom Score) in the different phases of the study are performed using Student's t-test.

Experimental Results:

The experimental results obtained using adenosine challenge indicated that the ozone-scavenging limonene treatment significantly and specifically relieved asthmatic stress in 6/11 asthmatics treated compared to negative control treatment with cineole. A much more pronounced effect, with approximately one order of magnitude improvement, was observed in severely asthmatic patients as compared to moderately asthmatic patients.

Conclusion: The presently disclosed experimental studies therefore demonstrate for the first time, using a highly realistic animal model, and using clinical trials in human patients, that compounds, such as limonene, which are capable of scavenging proinflammatory oxidants, such as ozone, can be used for effectively treating inflammation-associated medical conditions, such as asthma, in mammals in general and in humans in particular. Thus, the presently described results indicate that compounds capable of inhibiting oxidation by proinflammatory oxidants, such as ozone, at sites of inflammation, can be generally used as pharmacological agents for the effective prevention and treatment of inflammation-associated medical conditions in general. As such, the presently disclosed compounds and methods overcome numerous limitations of the prior art.

### Example 3

### Detection of Cellular Ozone Production Using Ozone-Sensitive Lyotropic Mesophases: Optimal Methods of Diagnosing Inflammation-Associated Diseases and of Identifying Candidate Anti-Inflammatory Compounds

As described above, production of oxidants, such as ozone, by activated immune effector cells, such as neutrophils, is a trigger of pathogenic inflammation in affected tissues in major inflammatory diseases, such as asthma, for which no optimal treatment or prophylaxis methods are available. There is therefore a clearly felt need for optimal methods of screening/characterizing compounds which are capable of inhibiting pathogenic oxidation mediated by oxidants, such as ozone, which are produced by activated immune effector cells, such as neutrophils. Such methods could be used to identify/design optimal drugs for treatment of inflammatory diseases such as asthma. However, to date, there are no optimal in-vitro methods for screening/characterizing optimal compounds capable of forming the basis of such drugs. Therefore in order to overcome such limitations of the prior art, the present inventors devised an optimal method of screening and characterizing such compounds, as described below.

Technological Concept:

Cromolyn sodium forms lyotropic mesophases in aqueous solutions at concentrations higher than approximately 7 percent (w/w). Cromolyn sodium undergoes rapid ozonolysis upon exposure to ozone, resulting in depletion of the cromolyn sodium concentration below the critical value needed to form a liquid crystalline phase. Since the mesophase, in contrast to an isotropic phase, is characterized by variety of optical (e.g., birefringence) and other easily monitored physical properties, this mesophase is a highly sensitive tool for detection of small quantities of ozone. In particular, the cromolyn sodium mesophase can detect emission of ozone by various cells, including mast cells, macrophages, neutrophils, basophils, eosinophils and other cells that are suspected producers of ozone. Emission of ozone is thus possible to detect at the level of a single cell. This approach is useful as a diagnostic tool to identify abnormal behavior of ozone producers (high/low levels), and to obtain early information concerning various diseases that involve ozone. Furthermore, this technique allows for the detection of ozone productivity by cells and by tissues, thus providing unique characterization opportunities of various medical conditions, characterization of genetically different populations, etc. Alternatively, the detection of ozone productivity can be performed using a cholesteric phase, which is achieved by contamination of a lyotropic liquid crystal, such as CS, with cholesterol or another unsaturated asymmetric compound. Depletion of the unsaturated contaminant by ozonolysis changes the mesophase from cholesteric to nematic, a change that is significantly visible by optical methods. This technology can be further used as a method of screening for ozone scavengers. Drug candidates that are capable of scavenging ozone better than a mesophase, particularly a mesophase formed by CS in aqueous buffer solutions, protect a lyotropic nematic or cholesteric CS mesophase from ozonolysis. Hence, screening for potential ozone scavengers is performed by adding small constant quantities of ozone to a CS mesophase after contaminating it with a given drug candidate, and thereafter automatically monitoring the optical properties of the mesophase, thus providing for a high throughput screening of large combinatorial libraries of ozone scavengers.

Materials and Methods:

Isolation of human neutrophils: Neutrophils were purified from freshly drawn whole blood as previously described (Markert et al., 1984. Methods Enzymol. 105:358). The isolation involved three basic steps: dextran sedimentation, lysis of contaminating red blood cells, and Ficoll-Hypaque density gradient centrifugation to separate neutrophils from platelets and mononuclear cells. Thus, a 6 percent Dextran 70 solution was added to citrate-treated human whole blood. After inversion and incubation at room temperature, total leukocytes were removed by a series of centrifugation steps, followed by hypotonic lysis of residual erythrocytes. Cells were subjected to centrifugation in a Ficoll Hypaque density gradient (Amersham Pharmacia) to further purify neutrophils. Prepared neutrophils were resuspended in Hank's Balanced Salt Solution (HBSS) or phosphate buffered saline (PBS), pH 7.4. The method was used to obtain a preparation containing over 99 percent granulocytes (90-95 percent neutrophils and the remainder eosinophils). The yield from 100 ml blood was approximately 100 million cells.

Activation of human neutrophils: Neutrophils can be activated to produce superoxide anions, ozone and other oxidants by a number of activating agents. The activating agent used was phorbol myristate acetate (PMA), which was stored as a stock solution in dimethyl sulfoxide (0.1 mg/ml) at −20 degrees centigrade. The neutrophils were activated for 10 minutes at 37 degrees centigrade with 1 microgram/ml PMA.

Preparation of cromolyn sodium lyotropic mesophases: Solutions of cromolyn sodium (white powder, Vitamed Inc., Israel) were dissolved in phosphate buffered saline [50 mM phosphate, 100 mM NaCl (pH 7.4)] at eight different concentrations between 5 and 12 percent (w/w). Samples were examined by a polarizing microscope and the minimal concentration that exhibited birefringence was found to be 6 percent CS.

Analysis of the effect of ozone on cromolyn sodium mesophases: Six solutions of cromolyn sodium in PBS (7-12 percent) were placed in narrow (5 mm) NMR tubes and ozone (10 percent in oxygen) was bubbled for 5 minutes through the solutions using a flexible Teflon tube. Loss of birefringence in all tubes was observed by a polarizing microscope. ¹H-NMR spectra of the resultant solutions indicated complete conversion of cromolyn sodium to oxidized products. Control experiments were carried out with hydrogen peroxide (30 percent) and sodium hypochlorite. After 8 hours at room temperature loss of birefringence was observed only with 7 percents and 8 percents cromolyn sodium with hydrogen peroxide. No effect was observed with hydrogen peroxide and more concentrated cromolyn sodium. No effect was observed with hypochlorite with any concentration of cromolyn sodium.

Examination of activated neutrophils in cromolyn sodium lyotropic mesophases: To 0.1 ml of a freshly purified solution of neutrophils, 0.001 ml of a PMA solution was added and the mixture was kept at room temperature for 5 minutes. 0.005 ml of the activated neutrophils solution was added to 0.1 ml of cromolyn sodium solutions (7, 8, and 9 percent). Samples of 0.005 ml were examined using a polarizing microscope. After 10 hours at room temperature, all three solutions were examined for birefringence.

Identification of candidate anti-inflammatory compounds: Candidate anti-inflammatory compounds are added to the mesophase at a concentration selected from nanomolar to milligram range, 1 million activated neutrophils are added to 1 ml of the mesophase-candidate compound mixture, and the capacity of the candidate anti-inflammatory compound to specifically inhibit degradation of the mesophase in the presence of the activated cells is determined. Compounds found to be capable of significantly inhibiting mesophase degradation constitute potential anti-inflammatory drugs. In order to enable detection of inhibition of mesophase degradation, candidate compounds which are tested are those which can react with ozone with a significantly higher reaction rate than that of the reaction of ozone with the mesophase.

Experimental Results:

Detection of ozone production by activated neutrophils using a cromolyn sodium lyotropic mesophase: After 10 hours at room temperature, solutions of 7, 8, or 9 percent cromolyn sodium in the presence of activated neutrophils were no longer birefringent, as determined using a polarizing microscope, therefore indicating significant loss of cromolyn sodium via ozonolysis by neutrophil produced ozone.

Conclusion: The above described results indicate that the presently disclosed method can be used for detecting with optimal sensitivity production of inflammation-associated oxidants, such as ozone, by immune cells such as activated neutrophils. By virtue of optimally enabling such detection, the presently disclosed method can be used for optimally diagnosing an inflammation-associated medical condition, such as asthma, which is mediated by such oxidants. Furthermore, since production of oxidants, such as ozone, by activated immune effector cells, such as neutrophils, is a trigger of pathogenic inflammation in affected tissues in major inflammatory diseases, such as asthma, the presently disclosed in-vitro system can be used to optimally identify candidate anti-inflammatory compounds for treatment of such medical conditions. As well, by providing an efficient and highly sensitive in-vitro system for detecting production of proinflammatory oxidants by immune cells, the presently disclosed method enables optimal high-throughput identification of such candidate anti-inflammatory compounds. Hence, the presently disclosed method overcomes numerous limitations of prior art methods of diagnosing inflammation-associated medical conditions, and of prior art methods of identifying candidate anti-inflammatory compounds.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, and patent applications mentioned in this specification are herein incorporated in their entirety by reference into the specification, to the same extent as if each individual publication, patent, or patent application was specifically and individually indicated to be incorporated herein by reference. In addition, citation or identification of any reference in this application shall not be construed as an admission that such reference is available as prior art to the present invention.

1-268. (canceled)

269. A method of treating a medical condition associated with inflammation in a subject in need thereof, the method comprising administering to the subject a therapeutically effective amount of at least one compound capable of inhibiting an activity and/or a formation of an oxidant associated with said inflammation,
with the proviso that when said medical condition is asthma, said at least one compound does not include any one of theophylline, enprofylline, pentoxifylline, lisofylline, 3-isobutyl-1-methylxanthine, caffeine, uric acid, khellin, cromolyn sodium, quercetin and nedocromil sodium.

270. The method of claim 269, wherein said administering is effected via inhalation of said compound.

271. The method of claim 269, wherein said inhibiting is by a stoichiometric reaction of said at least one compound and said oxidant, and/or is by a catalytic reaction of said at least one compound and said oxidant.

272. The method of claim 269, wherein said at least one compound is:
(i) capable of inhibiting a biochemical pathway that produces said oxidant;
(ii) capable of scavenging said oxidant;
(iii) substantially lipophilic;
(iv) substantially hydrophobic; and/or;
(v) substantially volatile.

273. The method of claim 269, wherein said oxidant is selected from the group consisting of a reactive oxygen species, ozone, hydrogen peroxide, a superoxide ion, a superoxide radical, a hydroxyl radical and a hypohalous acid.

274. The method of claim 272, wherein said scavenging is by a stoichiometric reaction of said at least one compound and said oxidant, and/or is by a catalytic reaction of said at least one compound and said oxidant.

275. The method of claim 272, wherein said at least one compound is selected from the group consisting of an alkene, an α,β-unsaturated carbonyl, a terpene, a xanthine, a chromone, an unsaturated fatty acid, an indigoid, an organic conductor and any derivative, analog or a pharmaceutically acceptable salt thereof.

276. The method of claim 272, wherein said at least one compound has a general Formula VII: 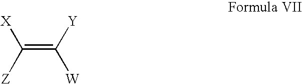
wherein each of X, Y, Z and W is independently selected from the group consisting of hydrogen, alkyl, alkenyl, hydroxyalkyl, trihaloalkyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azo, sulfonyl, sulfinyl, sulfonamide, phosphonyl, phosphinyl, phosphonium, ketone, ketoester, carbonyl, thiocarbonyl, ether, thiocarboxy, thioether, thiocarbamate, urea, thiourea, O-carbamyl, N-carbamyl, O-thiocarbamyl, N-thiocarbamyl, C-amido, N-amido, C-carboxy, O-carboxy, guanyl, guanidine and amino, or, alternatively, at least two of X, Y, Z and W form at least one five- or six-membered aromatic, heteroaromatic, alicyclic or heteroalicyclic ring,
or a pharmaceutically acceptable salt thereof.

277. The method of claim 276, wherein each of X, Y, Z and W is independently hydrogen or alkyl, or alternatively, two of X, Y, Z and W form a five- or six-membered alicyclic ring.

278. The method of claim 276, wherein at least two of X, Y, Z and W is an alkyl or alkenyl having at least 6 carbon atoms and at least one of said alkyl or alkenyl terminates with a C(═O)-L group or a pharmaceutically acceptable salt thereof, whereas L is selected from the group consisting of hydrogen, alkyl, cycloalkyl, aryl, hydroxy, alkoxy, aryloxy and amino.

279. The method of claim 276, wherein at least one of X, Y, Z and W is C-carboxy or a pharmaceutically acceptable salt thereof.

280. The method of claim 276, wherein at least two of X, Y, Z and W form at least one five- or six-membered aromatic, heteroaromatic, alicyclic and/or heteroalicyclic ring.

281. The method of claim 280, wherein said at least one compound has a general Formula I, II, III, V or VI: 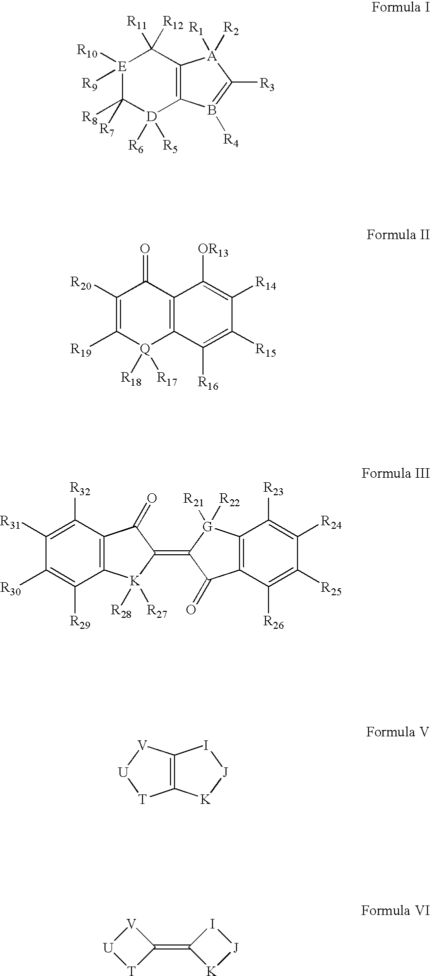
wherein:
each of A, D, E, Q, G and K is independently selected from the group consisting of carbon, nitrogen, oxygen and sulfur;
B is carbon or nitrogen;
each of I, K, T and V is independently selected from the group consisting of CR₃₇R₃₈, NR₃₉, O and S;
each of J and K is independnently selected from the group consisting of CR₄₀R₄₁, R₄₂R₄₃C—CR₄₄R₄₅, R₄₆R₄₇C—C(R₄₈R₄₉)—C(R₅₀R₅₁)—CR₅₂R₅₃ and R₅₄C═CR₅₅; and
each of R₁-R₅₅ is independently selected from the group consisting of hydrogen, lone pair electrons, alkyl, hydroxyalkyl, trihaloalkyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azo, sulfonyl, sulfinyl, sulfonamide, phosphonyl, phosphinyl, phosphonium, ketone, ketoester, carbonyl, thiocarbonyl, ether, thiocarboxy, thioether, thiocarbamate, urea, thiourea, O-carbamyl, N-carbamyl, O-thiocarbamyl, N-thiocarbamyl, C-amido, N-amido, C-carboxy, O-carboxy, guanyl, guanidine and amino, or, alternatively, at least two of R₁-R₄, at least two of R₅-R₁₂, at least two of R₁₃-R₁₆, at least two of R₁₇-R₂₀, at least two of R₂₁-R₂₆, at least two of R₂₇-R₃₂ and/or at least two of R₃₇-R₅₅ form at least one five- or six-membered aromatic, heteroaromatic, alicyclic or heteroalicyclic ring,
or a pharmaceutically acceptable salt thereof.

282. The method of claim 276, wherein at least one of X, Y, Z and W is an electron-donating group.

283. The method of claim 271, wherein said at least one compound is a metalloporphyrin or any derivative, analog or pharmaceutically acceptable salt thereof.

284. The method of claim 269, wherein said medical condition is selected from the group consisting of an inflammatory disease, an idiopathic inflammatory disease, a chronic inflammatory disease, an acute inflammatory disease, an allergic disease, an autoimmune disease, an infectious disease, an inflammatory malignant disease, an inflammatory transplantation-related disease, an inflammatory degenerative disease, an inflammatory injury, a disease associated with a hypersensitivity, an inflammatory cardiovascular disease, an inflammatory glandular disease, an inflammatory gastrointestinal disease, an inflammatory cutaneous disease, an inflammatory hepatic disease, an inflammatory neurological disease, an inflammatory musculo-skeletal disease, an inflammatory renal disease, an inflammatory reproductive disease, an inflammatory systemic disease, an inflammatory connective tissue disease, an inflammatory tumor, necrosis, an inflammatory implant-related disease and an inflammatory pulmonary disease.

285. A pharmaceutical composition identified for use in the treatment of a medical condition associated with inflammation, comprising, as an active ingredient, at least one compound capable of inhibiting an activity and/or formation of an oxidant associated with said inflammation, and a pharmaceutically acceptable carrier,
with the proviso that when said medical condition is asthma, said at least one compound does not include any one of theophylline, enprofylline, pentoxifylline, lisofylline, 3-isobutyl-1-methylxanthine, caffeine, uric acid, khellin, cromolyn sodium and nedocromil sodium.

286. The pharmaceutical composition of claim 285, packaged in a packaging material and identified in print, in or on said packaging material, for use in the treatment of said medical condition.

287. The pharmaceutical composition of claim 285, wherein said inhibiting is by a stoichiometric reaction of said at least one compound and said oxidant, and/or is by a catalytic reaction of said at least one compound and said oxidant.

288. The pharmaceutical composition of claim 285, wherein said at least one compound is:
(i) capable of inhibiting a biochemical pathway that produces said oxidant;
(ii) capable of scavenging said oxidant;
(iii) substantially lipophilic;
(iv) substantially hydrophobic; and/or;
(v) substantially volatile.

289. The pharmaceutical composition of claim 285, wherein said oxidant is selected from the group consisting of a reactive oxygen species, ozone, hydrogen peroxide, a superoxide ion, a superoxide radical, a hydroxyl radical and a hypohalous acid.

290. The pharmaceutical composition of claim 288, wherein said scavenging is by a stoichiometric reaction of said at least one compound and said oxidant, and/or is by a catalytic reaction of said at least one compound and said oxidant.

291. The pharmaceutical composition of claim 287, wherein said at least one compound is selected from the group consisting of an alkene, an α,β-unsaturated carbonyl, a terpene, a xanthine, a chromone, an unsaturated fatty acid, an indigoid, an organic conductor and any derivative or analog thereof.

292. The pharmaceutical composition of claim 288, wherein said at least one compound has a general Formula VII: 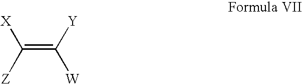
wherein each of X, Y, Z and W is independently selected from the group consisting of hydrogen, alkyl, alkenyl, hydroxyalkyl, trihaloalkyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azo, sulfonyl, sulfinyl, sulfonamide, phosphonyl, phosphinyl, phosphonium, ketoester, ketone, carbonyl, thiocarbonyl, ether, thiocarboxy, thioether, thiocarbamate, urea, thiourea, O-carbamyl, N-carbamyl, O-thiocarbamyl, N-thiocarbamyl, C-amido, N-amido, C-carboxy, O-carboxy, guanyl, guanidine and amino, or, alternatively, at least two of X, Y, Z and W form at least one five- or six-membered aromatic, heteroaromatic, alicyclic or heteroalicyclic ring,
or a pharmaceutically acceptable salt thereof,

293. The pharmaceutical composition of claim 287, wherein said at least one compound is a metalloporphyrin or any derivative, analog or pharmaceutically acceptable salt thereof.

294. The pharmaceutical composition of claim 285, wherein said medical condition is selected from the group consisting of an inflammatory disease, an idiopathic inflammatory disease, a chronic inflammatory disease, an acute inflammatory disease, an allergic disease, an autoimmune disease, an infectious disease, an inflammatory malignant disease, an inflammatory transplantation-related disease, an inflammatory degenerative disease, an inflammatory injury, a disease associated with a hypersensitivity, an inflammatory cardiovascular disease, an inflammatory glandular disease, an inflammatory gastrointestinal disease, an inflammatory cutaneous disease, an inflammatory hepatic disease, an inflammatory neurological disease, an inflammatory musculo-skeletal disease, an inflammatory renal disease, an inflammatory reproductive disease, an inflammatory systemic disease, an inflammatory connective tissue disease, an inflammatory tumor, necrosis, an inflammatory implant-related disease and an inflammatory pulmonary disease.

295. An inhalation device for use in the treatment of a medical condition associated with inflammation, comprising at least one compound capable of inhibiting an activity and/or formation of an oxidant associated with said inflammation and a respiratory delivery system,
with the proviso that when said medical condition is asthma, said at least one compound does not include any one of theophylline, enprofylline, pentoxifylline, lisofylline, 3-isobutyl-1-methylxanthine, caffeine, uric acid, khellin, quercetin, cromolyn sodium and nedocromil sodium.

296. The inhalation device of claim 295, wherein said respiratory delivery system is selected from the group consisting of a nebulizer inhaler, a dry powder inhaler, a metered dose inhaler, an oil warmer, a vaporizer and an atomizer.

297. The inhalation device of claim 295, wherein said inhibiting is by a stoichiometric reaction of said at least one compound and said oxidant, and/or is by a catalytic reaction of said at least one compound and said oxidant.

298. The inhalation device of claim 295, wherein said at least one compound is:
(i) capable of inhibiting a biochemical pathway that produces said oxidant;
(ii) capable of scavenging said oxidant;
(iii) substantially lipophilic;
(iv) substantially hydrophobic; and/or;
(v) substantially volatile.

299. The inhalation device of claim 295, wherein said oxidant is selected from the group consisting of a reactive oxygen species, ozone, hydrogen peroxide, a superoxide ion, a superoxide radical, a hydroxyl radical and a hypohalous acid.

300. The inhalation device of claim 298, wherein said scavenging is by a stoichiometric reaction of said at least one compound and said oxidant, and/or is by a catalytic reaction of said at least one compound and said oxidant.

301. The inhalation device of claim 297, wherein said at least one compound is selected from the group consisting of an alkene, an α,β-unsaturated carbonyl, a terpene, a xanthine, a chromone, an unsaturated fatty acid, an indigoid, an organic conductor and any derivative or analog thereof.

302. The inhalation device of claim 298, wherein said at least one compound has a general Formula VII: 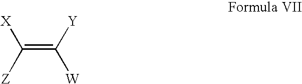
wherein each of X, Y, Z and W is independently selected from the group consisting of hydrogen, alkyl, alkenyl, hydroxyalkyl, trihaloalkyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, cyano, nitro, azo, sulfonyl, sulfinyl, sulfonamide, phosphonyl, phosphinyl, phosphonium, ketone, ketoester, carbonyl, thiocarbonyl, ether, thiocarboxy, thioether, thiocarbamate, urea, thiourea, O-carbamyl, N-carbamyl, O-thiocarbamyl, N-thiocarbamyl, C-amido, N-amido, C-carboxy, O-carboxy, guanyl, guanidine and amino, or, alternatively, at least two of X, Y, Z and W form at least one five- or six-membered aromatic, heteroaromatic, alicyclic or heteroalicyclic ring,
or a pharmaceutically acceptable salt thereof.

303. The inhalation device of claim 297, wherein said at least one compound is a metalloporphyrin or any derivative, analog or pharmaceutically acceptable salt thereof.

304. The inhalation device of claim 295, wherein said medical condition is selected from the group consisting of an inflammatory disease, an idiopathic inflammatory disease, a chronic inflammatory disease, an acute inflammatory disease, an allergic disease, an autoimmune disease, an infectious disease, an inflammatory malignant disease, an inflammatory transplantation-related disease, an inflammatory degenerative disease, an inflammatory injury, a disease associated with a hypersensitivity, an inflammatory cardiovascular disease, an inflammatory glandular disease, an inflammatory gastrointestinal disease, an inflammatory cutaneous disease, an inflammatory hepatic disease, an inflammatory neurological disease, an inflammatory musculo-skeletal disease, an inflammatory renal disease, an inflammatory reproductive disease, an inflammatory systemic disease, an inflammatory connective tissue disease, an inflammatory tumor, necrosis, an inflammatory implant-related disease and an inflammatory pulmonary disease.

305. A method of identifying a candidate compound for treating a medical condition associated with inflammation, the method comprising:
exposing a medium containing a compound in a mesophase state and a test compound to an oxidant, said compound in said mesophase state being susceptible to a degradation induced by said oxidant, wherein the medical condition is associated with an activity and/or formation of said oxidant; and
evaluating a capacity of said test compound to regulate said degradation, thereby identifying the candidate compound for treatment of the medical condition associated with inflammation.

306. The method of claim 305, wherein said evaluating is effected by measuring and/or characterizing a phase of matter of said medium, and/or an optical birefringence of said medium.

307. The method of claim 305, wherein said exposing is effected by contacting said medium with at least one cell which is capable of producing said oxidant.

308. The method of claim 305, wherein said compound in said mesophase state is a chromone derivative.

309. The method of claim 305, wherein said oxidant is selected from the group consisting of a reactive oxygen species, ozone, hydrogen peroxide, a superoxide ion, a superoxide radical, a hydroxyl radical and a hypohalous acid.

310. A method of qualifying a presence of an oxidant in a sample, the method comprising:
contacting the sample with a medium containing a compound in a mesophase state, said compound being susceptible to a degradation induced by the oxidant; and
evaluating a capacity of the sample to induce said degradation of said compound, thereby qualifying the presence of the oxidant in the sample.

311. The method of claim 310, wherein said evaluating is effected by measuring and/or characterizing a phase of matter of said medium, and/or an optical birefringence of said medium.

312. The method of claim 310, wherein said compound in said mesophase state is a chromone derivative.

313. The method of claim 310, wherein said oxidant is selected from the group consisting of a reactive oxygen species, ozone, hydrogen peroxide, a superoxide ion, a superoxide radical, a hydroxyl radical and a hypohalous acid.

314. The method of claim 310, wherein said sample comprises at least one cell.

315. A method of diagnosing a medical condition associated with inflammation in a subject, the method comprising:
contacting at least one cell being derived from the subject and capable of producing an oxidant, with a medium containing a compound in a mesophase state, said compound being susceptible to a degradation induced by said oxidant; and
evaluating a capacity of said at least one cell to induce said degradation of said compound, thereby diagnosing the medical condition associated with said inflammation.

316. The method of claim 315, wherein said evaluating is effected by measuring and/or characterizing a phase of matter of said medium and/or an optical birefringence of said medium.

317. The method of claim 315, wherein said compound in said mesophase state is a chromone derivative.

318. The method of claim 315, wherein said oxidant is selected from the group consisting of a reactive oxygen species, ozone, hydrogen peroxide, a superoxide ion, a superoxide radical, a hydroxyl radical and a hypohalous acid.
